(19) United States
(12) Patent Application Publication
(10) Pub. No.: US 2005/0235375 A1
(43) Pub. Date: Oct. 20, 2005

(54) Transcription factors of cereals

(76) Inventors: Wenqiong Chen, San Diego, CA (US); Steven P. Briggs, Del Mar, CA (US); Bret Cooper, La Jolla, CA (US); Jane Glazebrook, San Diego, CA (US); Stephen A. Goff, Research Triangle Park, NC (US); Fumiaki Katagiri, St. Paul, MN (US); Joel Kreps, San Diego, CA (US); Todd Moughamer, Research Triangle Park, NC (US); Nicolas J. Provart, Ontario (CA); Darrell Ricke, Research Triangle Park, NC (US); Tong Zhu, Research Triangle Park, NC (US)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(21) Appl. No.: 10/481,080

(22) PCT Filed: Jun. 21, 2002
(86) PCT No.: PCT/US02/19670

§ 371 Date: Jun. 4, 2004

Related U.S. Application Data
(60) Provisional application No. 60/300,112, filed on Jun. 22, 2001.
(60) Provisional application No. 60/325,377, filed on Sep. 27, 2001.
(60) Provisional application No. 60/370,428, filed on Apr. 4, 2002.

(51) Int. Cl. A01H 1/00; C12N 9/22; C07H 21/04; C12N 15/82; A01H 5/00; C12N 5/04

(52) U.S. Cl. 800/278; 800/320.2; 435/419; 435/468; 435/199; 536/23.6

(57) ABSTRACT

Polynucleotides encoding transcription factors of cereals and in particular rice are provided. Also provided are re-combinant vectors, expression cassettes, host cells and plants containing the polynucleotides. Methods for using the polynucleotides to alter resistance or tolerance of plants to stress, alter biological pathways, and alter gene expression are also provided.

### SPECIFCATION

This application is related to and claims priority of U.S. filed provisional applications U.S. Ser. No. 60/370,428, filed Apr. 4, 2002; U.S. Ser. No. 60/300,112 filed, Jun. 22, 2001; and U.S. Ser. No. 60/325,277 filed Sep. 26, 2001, each of which is incorporated by reference in its entirety for all purposes including, but not limited to, all text figures, tables, claims, sequence listings, supplemental tables, supplemental figures, appendices and material submitted on electronic media.

### REFERENCE TO MATERIAL SUBMITTED ON COMPACT DISC

The sequence listing accompanying this application is contained on compact disc pursuant to PCT Rules 89bis and 89ter, and PCT Administrative Rules part 8. The compact disc containing the sequence listing has been submitted in triplicate on compact disc labeled “Copy 1”, “Copy 2” and “Copy 3.” An additional copy labeled “CFR” has also been provided. Each compact disc contains text file named 70031_Seq_Lst which is 184 KB in size and which was created on Jun. 21, 2002.

### FIELD OF THE INVENTION

The present invention is in the field of plant biotechnology. In particular, the invention relates to nucleic acid molecules that comprise plant nucleotide sequences containing open reading frames encoding transcription factors.

### BACKGROUND OF THE INVENTION

The recently completed Arabidopsis genome sequencing project represents a major breakthrough for plant biology research (Arabidopsis Genome Initiative, 2001), as it provided all of the genetic code for the most popular model system of higher plants. A total of 25,498 genes were identified by gene prediction software, most of unknown function. Gene function can be assigned, however, through gene expression analysis in conjunction with forward or reverse genetics. By utilizing gene expression analysis, genes of unknown function can be associated with phenotypic or biochemical traits, and therefore potential functions can be assigned to these genes. The potential functions can be further confirmed by reverse genetics.

Plant development is precisely coordinated and regulated through the transcription and translation of different gene products in each cell. The expression level for each gene present in a cell not only reflects the physiological status of the cell, but also determines the range of different functions the cell can perform. Identification of genes expressed in a specific cell type, tissue, or developmental stage and the analysis of the abundance of their gene product can provide valuable insights into basic molecular processes.

For example, plants have developed various mechanisms to respond to different environmental and developmental stimuli. One type of response is through the activation or repression of gene expression. Plant transcription factors, as one of the final components in signal transduction pathways, play an important role in governing gene expression.

Thus, what is needed is the systematic identification of plant regulatory genes useful to control transcription of one or more genes in response to environmental and developmental stimuli.

### SUMMARY OF THE INVENTION

One aspect of the present invention provides isolated nucleic acid molecules (polynucleotides) comprising plant nucleotide sequences comprising an open reading frame encoding transcription factors. In one embodiment, the transcription factor comprises a cereal transcription factor. In another embodiment the transcription factor comprises a rice transcription factor. In another embodiment, the expression of the transcription factor is altered, either increased or decreased, in response to developmental or environmental stimuli, e.g., in response to biotic or abiotic stress, such as pathogen infection, or a shortage or excess of solar energy, water, nutrients, hormones, oxygen, carbon dioxide, salinity, temperature or pollution, for example, cold, salt or osmotic stress. As used herein, a “pathogen” includes bacteria, fungi, oomycetes, viruses, nematodes and insects, e.g., aphids (see Hammond-Kosack and Jones, 1997). The expression of a plant nucleotide sequence or a combination of nucleotide sequences encoding transcription factors of the invention may be useful to alter the phenotype of a plant, for example, to confer tolerance or resistance of a plant to one or more species of bacteria, nematode, fungi, oomycete, virus or insect and/or one or more abiotic stresses and/or to modulate or alter the expression of a plurality of genes regulated by the transcription factor, e.g., in a developmental and/or tissue specific manner.

Another aspect of the present invention provides a polynucleotide encoding a transcription factor comprising a sequence selected from the group consisting of SEQ ID NO: 1-70 or the complements thereof. In one embodiment, the polynucleotide is obtained from a monocot, for example a cereal, while in another embodiment, the polynucleotide is obtained from rice. A further aspect provides a polynucleotide that is substantially similar, and has at least 70%, 71%, 72%, 73%, 74%, 75%, 76%, 77%, 78%, 79%, 80%, 81%, 82%, 83%, 84%, 85%, 86%, 87%, 88%, 89%, at least 90%, 91%, 92%, 93%, 94%, 95%, 96%, 97%, 98%, and 99%, sequence identity to any one of SEQ ID NO.: 1-70 or the complements thereof. Included are fragments of any one of SEQ ID NO.: 1-70 that comprises a sequence encoding a polypeptide capable of acting as a transcription factor. Also included are polynucleotides and polynucleotide fragments of at least 7, at least 12, at least 15, at least 25, at least 50 or at least 100 nucleotides long that hybridizeto any one of SEQ ID NO.: 1-70 or the complements thereof under moderate to stringent or more highly stringent conditions.

An additional aspect provides a transcription factor polypeptide encoded by any one of SEQ ID NO.: 1-70 or a fragment thereof In one embodiment, the transcription factor comprises a monocot transcription factor, for example, a cereal transcription factor, while in another embodiment the transcription factor comprises a rice transcription factor. Also included within the scope of the present inventive discovery are polypeptides comprising transcription factors having amino acid sequences substantially similar to the sequences encoded by SEQ ID NO.: 1-70. Typically, such amino acid sequences have at least 70%, 71%, 72%, 73%, 74%, 75%, 76%, 77%, 78%, 79%, 80%, 81%, 82%, 83%, 84%, 85%, 86%, 87%, 88%, 89%, at least 90%, 91%, 92%, 93%, 94%, 95%, 96%, 97%, 98%, and 99%, sequence identity to the amino acid sequences encoded by SEQ ID NO.: 1-70. Since it is well known in the art that the degeneracy of the genetic code allows multiple nucleotide sequences to encode the same amino acid sequence, the present invention includes any polynucleotide encoding the same or substantially similar amino acid sequence to the amino acid sequence encoded by any one of SEQ ID NO: 1-70 or functional fragment thereof.

In one embodiment, the transcription factors of the present invention can be classified into known transcription factor families. Such families include, but are not limited to, zinc finger proteins, Myb transcription factors, WRKY transcription factors, IAA7 transcription factors, AP2/EREBP type transcription factors, leucine zipper transcription factors, bZIP transcription factors and homeo-domain transcription factors.

The transcription factors of the present invention include those whose expression is altered by particular stimuli. For example, transcription factors whose expression is altered, e.g. induced or repressed, by abiotic or biotic stress, or by the presence of a chemical compound. Non-limiting examples of abiotic stress include cold stress, heat stress, salt stress, drought stress, and mechanical injury. Non-limiting examples of biotic stress include infection with a pathogen such as a bacterial, viral or fungal pathogen. In another embodiment, the transcription factors are tissue specific transcription factors.

The present invention further provides an expression cassette or a vector containing the nucleic acid molecule comprising an open reading frame encoding a transcription factor of the invention. In the case of an expression cassette, the open reading frame is operably linked to a promoter. The vector may be a cloning or an expression vector. Further, the vector may be a plasmid. Such cassettes or vectors, when present in a plant, plant cell or plant tissue result in transcription of the linked open reading frames in the plant. The expression cassettes or vectors of the invention may optionally include other regulatory sequences, e.g., transcription terminator sequences, operator, repressors binding sites, transcription factor binding sites, and/or an enhancers and may be contained in a host cell. The expression cassette or vector may augment the genome of a transformed plant, or cells there of (including protoplast), or may be maintained extrachromosomally. In one embodiment the expression cassette or vector may be a Ti plasmid and be contained in an Agrobacterium tumefaciens cell. The expression cassette may further be carried on a microparticle, wherein the microparticle is suitable for ballistic transformation of a plant cell; or it may be contained in a plant cell or protoplast. Further still, the expression cassette can be contained in a transformed plant or cells thereof and the plant may be a dicot or a monocot. In particular, the plant may be a cereal and more particularly a rice plant.

The invention also provides sense and anti-sense nucleic acid molecules corresponding to the open reading frames encoding transcription factors identified herein, as well as their orthologs. Also provided are expression cassettes, e.g., recombinant vectors, and host cells, comprising the nucleic acid molecules of the invention, e.g., one that comprises a nucleotide sequence that encodes a transcription factor described herein.

The present invention further provides a method of augmenting the genetic complement of a plant by contacting plant cells with a nucleic acid molecule of the invention, e.g., one isolatable or obtained from a plant encoding a polypeptide that is substantially similar to a polypeptide encoded by SEQ ID NO.: 1-70, so as to yield transformed plant cells; and regenerating the transformed plant cells to provide a differentiated transformed plant, wherein the differentiated transformed plant expresses the nucleic acid molecule in at least one cell of the plant. The nucleic acid molecule may be present in the nucleus, chloroplast, mitochondria and/or other plastid of the cells of the plant. The present invention also provides a transgenic plant prepared by this method, a seed from such a plant, and progeny plants from such a plant including hybrids and inbreds. Preferred transgenic plants include, but are not limited to, transgenic maize, soybean, barley, alfalfa, sunflower, canola, cotton, peanut, sorghum, tobacco, sugarbeet, rice, wheat, rye, turfgrass, millet, sugarcane, tomato, or potato.

The invention also provides a method of plant breeding, e.g., to prepare a crossed fertile transgenic plant. The method comprises crossing a fertile transgenic plant comprising a particular nucleic acid molecule of the invention with itself or with a second plant, e.g., one lacking the particular nucleic acid molecule, to prepare the seed of a crossed fertile transgenic plant comprising the particular nucleic acid molecule. The seed is then planted to obtain a crossed fertile transgenic plant. The plant may be a monocot or a dicot. In one embodiment, the plant is a cereal and more particularly a rice plant.

The crossed fertile transgenic plant may have the particular nucleic acid molecule inherited through a female parent or through a male parent. The second plant may be an inbred plant. The crossed fertile transgenic may be a hybrid. Also included within the present invention are seeds of any of these crossed fertile transgenic plants.

The various breeding steps are characterized by well-defined human intervention such as selecting the lines to be crossed, directing pollination of the parental lines, or selecting appropriate progeny plants. Depending on the desired properties, different breeding measures are taken. The relevant techniques are well known in the art and include, but are not limited to, hybridization, inbreeding, backcross breeding, multiline breeding, variety blend, interspecific hybridization, aneuploid techniques, etc. Hybridization techniques also include the sterilization of plants to yield male or female sterile plants by mechanical, chemical or biochemical means. Cross pollination of a male sterile plant with pollen of a different line assures that the genome of the male sterile but female fertile plant will uniformly obtain properties of both parental lines. Thus, the transgenic plants according to the invention can be used for the breeding of improved plant lines that for example increase the effectiveness of conventional methods such as herbicide or pesticide treatment, or allow to dispense with said methods, due to their modified genetic properties.

Another aspect of the invention provides a method of altering gene expression that in turn may alter the phenotype of a plant by expression of a transcription factor or combination of transcription factors of the present invention. In this aspect, a recombinant expression cassette comprising a promoter operably linked to a polynucleotide encoding a transcription factor of the present invention, or a functional fragment thereof, is introduced into a plant using any method known in the art. A variety of promoters can be used including those described herein, for example, constitutive, inducible, tissue specific, or developmentally regulated promoters.

In this method, the expressed transcription factor binds to a gene in the plant resulting in altered, e.g. inducing or repressing, expression of the gene. This change in gene expression can ultimately lead to a desirable change in the phenotype of the plant. The gene upon which the transcription factor acts, can be an endogenous gene or an introduced gene or transgene.

In one embodiment, transcription factors directed to particular pathways are used, for example, to provide resistance or tolerance of a stress in a plant. The sequence encoding the transcription factor may be overexpressed individually, in the sense or antisense orientation, or in combination with other sequences to confer desirable characteristics, such as, improved disease or stress resistance or tolerance to a plant relative to a plant that does not comprise and/or express the sequence. The overexpression may be constitutive, or it may be desirable to express the desired gene(s), i.e., an effector gene, in a tissue-specific manner or from an inducible promoter, including a promoter that is responsive to external stimuli, such as chemical application, or to pathogen infection, e.g., so as to avoid possible deleterious effects on plant growth if the effector gene(s) is constitutively expressed. In one embodiment of the invention, the promoter employed may be one that is rapidly and transiently and/or highly transcribed after stress, e.g., after pathogen infection.

Another aspect of the invention provides plants wherein the cells, genome, or components there of have been augmented with the polynucleotides of the present invention. Said augmentation is especially advantageous wherein the endogenous polynucleotides corresponding to any one of SEQ ID NO.: 1-70 have been disrupted so as to result in a loss, decrease, or alteration of function of the transcription factor encoded by the polynucleotide. In an alternative embodiment, the plant is transformed with a construct, the expression of which produces a product that interacts with the transcription factor encoded by any one of SEQ ID NO.: 1-70 and alters its function, for example, prevents binding of the transcription factor to its target DNA.

A further aspect provides a polynucleotide of at least 5, at least 10, at least 15, at least 25, at least 50 or at least 100 nucleotides in length that is complementary to one of SEQ ID NO.: 1-70 (test sequence) and which hybridizes under low, moderate, stringent or highly stringent conditions with any of SEQ ID NO.: 1-70 as well as RNA encoded by any of SEQ ID NO.: 1-70, when the hybridization is performed under stringent conditions, either the test or nucleic acid molecule of the invention may be supported, e.g., on a membrane or DNA chip. Thus, either a denatured test or nucleic acid molecule of the invention is first bound to a support and hybridization is effected for a specified period of time at a temperature of, e.g., between 55 and 70° C., in double strength citrate buffered saline (SC) containing 0.1% SDS followed by rinsing of the support at the same temperature, but with a buffer having a reduced SC concentration.

Depending upon the degree of stringency required, such reduced concentration buffers are typically single strength SC containing 0.1% SDS, half strength SC containing 0.1% SDS and one-tenth strength SC containing 0.1% SDS. Such polynucleotides can be used to identify orthologs of the transcription factors of the present invention especially when such polynucleotides are used in expression analysis to identify nucleotide sequences which share sequence homology and expression profiles similar to the transcription factors of the present invention.

A method to shuffle the nucleic acids of the invention is also provided. This method involves fragmentation of a nucleic acid corresponding to SEQ ID NO.: 1-70, the orthologs thereof, and the corresponding genes, followed by religation. This method allows for the production of polypeptides having altered activity relative to the native form of the polypeptide. Accordingly, the invention provides cells and transgenic plants containing nucleic acid segments, produced through shuffling, that encode polypeptides having altered activity relative to the corresponding native polypeptide.

A computer readable medium containing the nucleic acid sequences of the invention as well as methods of use for the computer readable medium are provided. This medium allows a nucleic acid segment corresponding to SEQ ID NO.: 1-70, the complements thereof, or the orthologs thereof to be used as a reference sequence to search against databases. This medium also allows for computer-based manipulation of a nucleic acid sequence corresponding to any one of SEQ ID NO.: 1-70, the complements thereof, or the orthologs thereof, and the corresponding gene and polypeptide encoded by the nucleic acid sequence.

The invention further provides a method for marker-assisted breeding to select for plants having advantages associated with expression of particular transcription factors disclosed herein. The method involves contacting plant DNA or cDNA with a probe corresponding to a nucleic acid sequence in the sequence listing, or the orthologs thereof, and the corresponding genes, or a portion thereof which hybridizes under moderate stringency conditions to a gene corresponding to one of SEQ ID NO.: 1-70, so as to form a duplex and detecting or determining the presence or amount of the duplex. The amount or presence of the duplex is indicative of the presence or expression of particular transcription factors.

Further aspects provide host cells comprising recombinant constructs containing any one of SEQ ID NO.: 1-70 or the complement thereof. Such host cells can be bacterial cells, animal cells, yeast cells or plant cells.

### DETAILED DESCRIPTION

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

All publications, patents, patent applications, public databases, public database entries, and other references cited in this application are herein incorporated by reference in their entirety as if each individual publication, patent, patent application, public database, public database entries, or other reference were specifically and individually indicated to be incorporated by reference.

The term “nucleic acid” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., 1991; Ohtsuka et al., 1985; Rossolini et al. 1994).

A “nucleic acid fragment” is a fraction of a given nucleic acid molecule. In higher plants, deoxyribonucleic acid (DNA) is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural or altered nucleotide bases capable of incorporation into DNA or RNA polymers. The terms “nucleic acid” or “nucleic acid sequence” may also be used interchangeably with gene, cDNA, DNA and RNA encoded by a gene.

“Genome” refers to the complete genetic material of an organism, specifically a plant, in particular, nuclear genetic material but inclusive of plastid genetic material.

The term “gene” is used broadly to refer to any segment of nucleic acid associated with a biological function. Thus, genes include coding sequences and/or the regulatory sequences required for their expression. For example, gene refers to a nucleic acid fragment that expresses mRNA or functional RNA, or encodes a specific protein, and which includes regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

The term “native” or “wild type” gene refers to a gene that is present in the genome of an untransformed cell.

A “marker gene” encodes a selectable or screenable trait.

The term “chimeric gene” refers to any gene that contains 1) DNA sequences, including regulatory and coding sequences, that are not found together in nature, or 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature.

“Trans-activation” refers to switching on of gene expression or replicon replication by the expression of another (regulatory) gene in trans.

A “transgene” refers to a gene that has been introduced into the genome by transformation and is stably maintained. Transgenes may include, for example, genes that are either heterologous or homologous to the genes of a particular plant to be transformed. Additionally, transgenes may comprise native genes inserted into a non-native organism, or chimeric genes. The term “endogenous gene” refers to a native gene in its natural location in the genome of an organism. A “foreign” gene refers to a gene not normally found in the host organism but that is introduced by gene transfer. “Transcription Stop Fragment” refers to nucleotide sequences that contain one or more regulatory signals, such as polyadenylation signal sequences, capable of terminating transcription. Examples include the 3′non-regulatory regions of genes encoding nopaline synthase and the small subunit of ribulose bisphosphate carboxylase.

“Chimeric trans-acting replication gene” refers either to a replication gene in which the coding sequence of a replication protein is under the control of a regulated plant promoter other than that in the native viral replication gene, or a modified native viral replication gene, for example, in which a site specific sequence(s) is inserted in the 5′ transcribed but untranslated region. Such chimeric genes also include insertion of the known sites of replication protein binding between the promoter and the transcription start site that attenuates transcription of viral replication protein genes.

“Antisense inhibition” refers to the production of antisense RNA transcripts capable of suppressing the expression of protein from an endogenous gene or a transgene.

“Co-suppression” and “transwitch” each refer to the production of sense RNA transcripts capable of suppressing the expression of identical or substantially similar transgene or endogenous genes (U.S. Pat. No. 5,231,020).

“Gene silencing” refers to homology-dependent suppression of viral genes, transgenes, or endogenous nuclear genes. Gene silencing may be transcriptional, when the suppression is due to decreased transcription of the affected genes, or post-transcriptional, when the suppression is due to increased turnover (degradation) of RNA species homologous to the affected genes (English et al., 1996). Gene silencing includes virus-induced gene silencing (Ruiz et al. 1998).

“Silencing suppressor” gene refers to a gene whose expression leads to counteracting gene silencing and enhanced expression of silenced genes. Silencing suppressor genes may be of plant, non-plant, or viral origin. Examples include, but are not limited to HC-Pro, P1-HC-Pro, and 2b proteins. Other examples include one or more genes in TGMV-B genome.

“Replication gene” refers to a gene encoding a viral replication protein. In addition to the ORF of the replication protein, the replication gene may also contain other overlapping or non-overlapping ORF(s), as are found in viral sequences in nature. While not essential for replication, these additional ORFs may enhance replication and/or viral DNA accumulation. Examples of such additional ORFs are AC3 and AL3 in ACMV and TGMV geminiviruses, respectively.

“Germline cells” refer to cells that are destined to be gametes and whose genetic material is heritable.

“Chromosomally-integrated” refers to the integration of a foreign gene or DNA construct into the host DNA by covalent bonds. Where genes are not “chromosomally integrated” they may be “transiently expressed.” Transient expression of a gene refers to the expression of a gene that is not integrated into the host chromosome but functions independently, either as part of an autonomously replicating plasmid or expression cassette, for example, or as part of another biological system such as a virus.

A “functional fragment” in reference to any one of SEQ ID NO. 1-70, refers to a portion of any of SEQ ID NO. 1-70 which encodes a polypeptide that is capable of binding to a nucleic acid molecule and altering transcription of that molecule, that is can act as a transcription factor.

The terms “heterologous DNA sequence,” “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

“Homologous to” in the context of nucleotide sequence identity refers to the similarity between the nucleotide sequence of two nucleic acid molecules or between the amino acid sequences of two protein molecules. Estimates of such homology are provided by either DNA-DNA or DNA-RNA hybridization under conditions of stringency as is well understood by those skilled in the art (as described in Haines and Higgins (eds.), Nucleic Acid Hybridization, IRL Press, Oxford, U.K.), or by the comparison of sequence similarity between two nucleic acids or proteins.

The term “substantially similar” refers to nucleotide and amino acid sequences that represent functional and/or structural equivalents of the sequences disclosed herein. For example, altered nucleotide sequences which simply reflect the degeneracy of the genetic code but nonetheless encode amino acid sequences that are identical to a particular amino acid sequence are substantially similar to the particular sequences. In addition, amino acid sequences that are substantially similar to a particular sequence are those wherein overall amino acid identity is at least 70% or greater to the instant sequences. Modifications that result in equivalent nucleotide or amino acid sequences are well within the routine skill in the art. Moreover, the skilled artisan recognizes that equivalent nucleotide sequences encompassed by this invention can also be defined by their ability to hybridize, under low, moderate and/or high stringency conditions (e.g., 0.1×SSC, 0.1% SDS, 65° C.), with the novel nucleotide sequences that are disclosed herein.

An “oligonucleotide” corresponding to a nucleotide sequence of the invention, e.g., for use in probing or amplification reactions, may be about 30 or fewer nucleotides in length (e.g., 5, 7, 9, 12, 15, 18, 20, 21 or 24, or any number between 5 and 30). Generally specific primers are upwards of 14 nucleotides in length. For optimum specificity and cost effectiveness, primers of 16 to 24 nucleotides in length are useful. Those skilled in the art are well versed in the design of primers for use processes such as PCR. If required, probing can be done with entire restriction fragments of the gene disclosed herein which may be 100's or even 1000's of nucleotides in length.

“Coding sequence” refers to a DNA or RNA sequence that codes for a specific amino acid sequence and excludes the non-coding sequences. It may constitute an “uninterrupted coding sequence”, i.e., lacking an intron, such as in a cDNA or it may include one or more introns bounded by appropriate splice junctions. An “intron” is a sequence of RNA which is contained in the primary transcript but which is removed through cleavage and re-ligation of the RNA within the cell to create the mature mRNA that can be translated into a protein.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

The nucleotide sequences of the invention can be introduced into any plant. The genes to be introduced can be conveniently used in expression cassettes for introduction and expression in any plant of interest. Such expression cassettes will comprise the transcriptional initiation region of the invention linked to a nucleotide sequence of interest. Suitable promoters include constitutive, tissue-specific, developmental-specific, inducible and/or viral promoters. Such an expression cassette may be provided with a plurality of restriction sites for insertion of the gene of interest to be under the transcriptional regulation of the regulatory regions.

The expression cassette may additionally contain selectable marker genes. The cassette will include in the 5′-3′ direction of transcription, a transcriptional and translational initiation region, a DNA sequence of interest, and a transcriptional and translational termination region functional in plants. The termination region may be native with the transcriptional initiation region, may be native with the DNA sequence of interest, or may be derived from another source. Convenient termination regions are available from the Ti-plasmid of A. tumefaciens, such as the octopine synthase and nopaline synthase termination regions. See also, Guerineau et al., 1991; Proudfoot, 1991; Sanfacon et al., 1991; Mogen et al., 1990; Munroe et al., 1990; Ballas et al., 1989; Joshi et al., 1987.

A “functional RNA” refers to an antisense RNA, ribozyme, or other RNA that is not translated.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences which may be a combination of synthetic and natural sequences. As is noted above, the term “suitable regulatory sequences” is not limited to promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency (Turner et al., 1995).

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., 1989.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

The term “intracellular localization sequence” refers to a nucleotide sequence that encodes an intracellular targeting signal. An “intracellular targeting signal” is an amino acid sequence that is translated in conjunction with a protein and directs it to a particular sub-cellular compartment. “Endoplasmic reticulum (ER) stop transit signal” refers to a carboxy-terminal extension of a polypeptide, which is translated in conjunction with the polypeptide and causes a protein that enters the secretory pathway to be retained in the ER. “ER stop transit sequence” refers to a nucleotide sequence that encodes the ER targeting signal. Other intracellular targeting sequences encode targeting signals active in seeds and/or leaves and vacuolar targeting signals.

The terms “protein,” “peptide” and “polypeptide” are used interchangeably herein.

The term “mature” protein refers to a post-translationally processed polypeptide without its signal peptide. “Precursor” protein refers to the primary product of translation of an mRNA. “Signal peptide” refers to the amino terminal extension of a polypeptide, which is translated in conjunction with the polypeptide forming a precursor peptide and which is required for its entrance into the secretory pathway. The term “signal sequence” refers to a nucleotide sequence that encodes the signal peptide.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers.

Accordingly, an “enhancer” is a DNA sequence which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors which control the effectiveness of transcription initiation in response to physiological or developmental conditions.

The “initiation site” is the position surrounding the first nucleotide that is part of the transcribed sequence, which is also defined as position +1. With respect to this site all other sequences of the gene and its controlling regions are numbered. Typically downstream sequences (i.e., further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Operably-linked” refers to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”. Examples of methods of transformation of plants and plant cells include Agrobacterium-mediated transformation (De Blaere et al., 1987) and particle bombardment technology (Klein et al. 1987; U.S. Pat. No. 4,945,050). Whole plants may be regenerated from transgenic cells by methods well known to the skilled artisan (see, for example, Fromm et al., 1990).

“Transformed”, “transgenic”, and “recombinant” refer to a host organism such as a bacterium or a plant into which a heterologous nucleic acid molecule has been introduced. For example, “transformed,” “transformant,” and “transgenic” plants or calli have been through the transformation process and contain a foreign gene integrated into their chromosome. The term “untransformed” refers to normal plants that have not been through the transformation process.

“Transiently transformed” refers to cells in which transgenes and foreign DNA have been introduced (for example, by such methods as Agrobacterium-mediated transformation or biolistic bombardment), but not selected for stable maintenance.

“Stably transformed” refers to cells that have been selected and regenerated on a selection media following transformation.

“Transient expression” refers to expression in cells in which a virus or a transgene is introduced by viral infection or by such methods as Agrobacterium-mediated transformation, electroporation, or biolistic bombardment, but not selected for its stable maintenance.

“Genetically stable” and “heritable” refer to genetic elements that are stably maintained in the plant and stably inherited by progeny through successive generations.

“Primary transformant” and “T0 generation” refer to transgenic plants that are of the same genetic generation as the tissue which was initially transformed (i.e., not having gone through meiosis and fertilization since transformation).

“Secondary transformants” and the “T1, T2, T3, etc. generations” refer to transgenic plants derived from primary transformants through one or more meiotic and fertilization cycles. They may be derived by self-fertilization of primary or secondary transformants or crosses of primary or secondary transformants with other transformed or untransformed plants.

“Constitutive expression” refers to expression using a constitutive or regulated promoter. “Conditional” and “regulated expression” refer to expression controlled by a regulated promoter.

“Constitutive promoter” refers to a promoter that is able to express the open reading frame (ORF) that it controls in all or nearly all of the plant tissues during all or nearly all developmental stages of the plant. Each of the transcription-activating elements do not exhibit an absolute tissue-specificity, but mediate transcriptional activation in most plant parts at a level of ≧1% of the level reached in the part of the plant in which transcription is most active.

“Regulated promoter” refers to promoters that direct gene expression not constitutively, but in a temporally and/or spatially-regulated manner, and includes both tissue-specific and inducible promoters. It includes natural and synthetic sequences as well as sequences which may be a combination of synthetic and natural sequences. Different promoters may direct the expression of a gene in different tissues or cell types, or at different stages of development, or in response to different environmental conditions. New promoters of various types useful in plant cells are constantly being discovered, numerous examples may be found in the compilation by Okamuro et al. (1989). Typical regulated promoters useful in plants include but are not limited to safener-inducible promoters, promoters derived from the tetracycline-inducible system, promoters derived from salicylate-inducible systems, promoters derived from alcohol-inducible systems, promoters derived from glucocorticoid-inducible system, promoters derived from pathogen-inducible systems, and promoters derived from ecdysome-inducible systems.

“Tissue-specific promoter” refers to regulated promoters that are not expressed in all plant cells but only in one or more cell types in specific organs (such as leaves or seeds), specific tissues (such as embryo or cotyledon), or specific cell types (such as leaf parenchyma or seed storage cells). These also include promoters that are temporally regulated, such as in early or late embryogenesis, during fruit ripening in developing seeds or fruit, in fully differentiated leaf, or at the onset of senescence.

“Inducible promoter” refers to those regulated promoters that can be turned on in one or more cell types by an external stimulus, such as a chemical, light, hormone, stress, or a pathogen.

“Expression” refers to the transcription and/or translation of an endogenous gene, ORF or portion thereof, or a transgene in plants. For example, in the case of antisense constructs, expression may refer to the transcription of the antisense DNA only. In addition, expression refers to the transcription and stable accumulation of sense (mRNA) or functional RNA. Expression may also refer to the production of protein.

“Specific expression” is the expression of a gene products that is limited to one or a few plant tissues (spatial limitation) and/or to one or a few plant developmental stages (temporal limitation). It is acknowledged that a true specificity exists: promoters seem to prefer to be switched on in some tissues, while in other tissues there is no or only little activity. This phenomenon is known as leaky expression. However, with specific expression in this invention is meant preferable expression in one or a few plant tissues.

The term “average expression” is used here as the average level of expression found in all lines that do express detectable amounts of reporter gene, so leaving out of the analysis plants that do not express any detectable reporter mRNA or protein.

“Non-specific expression” refers to constitutive expression or low level, basal (‘leaky’) expression in nondesired cells or tissues from a ‘regulated promoter’.

“Altered levels” refers to the level of expression in transgenic organisms that differs from that of normal or untransformed organisms.

“Overexpression” refers to the level of expression in transgenic cells or organisms that exceeds levels of expression in normal or untransformed (nontransgenic) cells or organisms.

The “expression pattern” of a promoter (with or without enhancer) is the pattern of expression levels which shows where in the plant and in what developmental stage transcription is initiated by said promoter. Expression patterns of a set of promoters are said to be complementary when the expression pattern of one promoter shows little overlap with the expression pattern of the other promoter. The level of expression of a promoter can be determined by measuring the ‘steady state’ concentration of a standard transcribed reporter mRNA. This measurement is indirect since the concentration of the reporter mRNA is dependent not only on its synthesis rate, but also on the rate with which the mRNA is degraded. Therefore, the steady state level is the product of synthesis rates and degradation rates. The rate of degradation can, however, be considered to proceed at a fixed rate when the transcribed sequences are identical, and thus this value can serve as a measure of synthesis rates. When promoters are compared in this way techniques available to those skilled in the art are hybridization S1-RNAse analysis, northern blots and competitive RT-PCR. This list of techniques in no way represents all available techniques, but rather describes commonly used procedures used to analyze transcription activity and expression levels of mRNA.

A commonly used procedure to analyze expression patterns and levels is through determination of the ‘steady state’ level of protein accumulation in a cell by using a reporter gene. Commonly used candidates for the reporter gene, known to those skilled in the art are β-glucuronidase (GUS), chloramphenicol acetyl transferase (CAT) and proteins with fluorescent properties, such as green fluorescent protein (GFP) from Aequora Victoria. In principle, however, many proteins are suitable for this purpose, provided the protein does not interfere with essential plant functions.

Two principal methods for the control of expression are known, viz.: overexpression and underexpression. Overexpression can be achieved by insertion of one or more than one extra copy of the selected gene. It is, however, not unknown for plants or their progeny, originally transformed with one or more than one extra copy of a nucleotide sequence, to exhibit the effects of underexpression as well as overexpression. For underexpression there are two principle methods which are commonly referred to in the art as “antisense downregulation” and “sense downregulation” (sense downregulation is also referred to as “cosuppression”). Generically these processes are referred to as “gene silencing”. Both of these methods lead to an inhibition of expression of the target gene.

Obtaining sufficient levels of transgene expression in the appropriate plant tissues is an important aspect in the production of genetically engineered crops. Expression of heterologous DNA sequences in a plant host is dependent upon the presence of an operably linked promoter that is functional within the plant host. Choice of the promoter sequence will determine when and where within the organism the heterologous DNA sequence is expressed.

Furthermore, it is contemplated that promoters combining elements from more than one promoter may be useful. For example, U.S. Pat. No. 5,491,288 discloses combining a Cauliflower Mosaic Virus promoter with a histone promoter. Thus, the elements from the promoters disclosed herein may be combined with elements from other promoters.

Promoters that are useful for plant transgene expression include those that are inducible, viral, synthetic, constitutive (Odell et al., 1985), temporally regulated, spatially regulated, tissue-specific, and spatio-temporally regulated.

Where expression in specific tissues or organs is desired, tissue-specific promoters may be used. In contrast, where gene expression in response to a stimulus is desired, inducible promoters are the regulatory elements of choice. Where continuous expression is desired throughout the cells of a plant, constitutive promoters are utilized. Additional regulatory sequences upstream and/or downstream from the core promoter sequence may be included in expression constructs of transformation vectors to bring about varying levels of expression of heterologous nucleotide sequences in a transgenic plant.

The choice of promoter will vary depending on the temporal and spatial requirements for expression, and also depending on the target species. In some cases, expression in multiple tissues is desirable. While in others, tissue-specific, e.g., leaf-specific, seed-specific, petal-specific, anther-specific, or pith-specific, expression is desirable. Although many promoters from dicotyledons have been shown to be operational in monocotyledons and vice versa, ideally dicotyledonous promoters are selected for expression in dicotyledons, and monocotyledonous promoters for expression in monocotyledons. However, there is no restriction to the provenance of selected promoters; it is sufficient that they are operational in driving the expression of the nucleotide sequences in the desired cell.

These promoters include, but are not limited to, constitutive, inducible, temporally regulated, developmentally regulated, spatially-regulated, chemically regulated, stress-responsive, tissue-specific, viral and synthetic promoters. Promoter sequences are known to be strong or weak. A strong promoter provides for a high level of gene expression, whereas a weak promoter provides for a very low level of gene expression. An inducible promoter is a promoter that provides for the turning on and off of gene expression in response to an exogenously added agent, or to an environmental or developmental stimulus. A bacterial promoter such as the P_{tac} promoter can be induced to varying levels of gene expression depending on the level of isothiopropylgalactoside added to the transformed bacterial cells. An isolated promoter sequence that is a strong promoter for heterologous nucleic acid is advantageous because it provides for a sufficient level of gene expression to allow for easy detection and selection of transformed cells and provides for a high level of gene expression when desired.

Within a plant promoter region there are several domains that are necessary for full function of the promoter. The first of these domains lies immediately upstream of the structural gene and forms the “core promoter region” containing consensus sequences, normally 70 base pairs immediately upstream of the gene. The core promoter region contains the characteristic CAAT and TATA boxes plus surrounding sequences, and represents a transcription initiation sequence that defines the transcription start point for the structural gene.

The presence of the core promoter region defines a sequence as being a promoter: if the region is absent, the promoter is non-functional. Furthermore, the core promoter region is insufficient to provide full promoter activity. A series of regulatory sequences upstream of the core constitute the remainder of the promoter. The regulatory sequences determine expression level, the spatial and temporal pattern of expression and, for an important subset of promoters, expression under inductive conditions (regulation by external factors such as light, temperature, chemicals, hormones).

A range of naturally-occurring promoters are known to be operative in plants and have been used to drive the expression of heterologous (both foreign and endogenous) genes in plants: for example, the constitutive 35S cauliflower mosaic virus (CaMV) promoter, the ripening-enhanced tomato polygalacturonase promoter (Bird et al., 1988), the E8 promoter (Diekman & Fischer, 1988) and the fruit specific 2A1 promoter (Pear et al., 1989) and many others, e.g., U2 and U5 snRNA promoters from maize, the promoter from alcohol dehydrogenase, the Z4 promoter from a gene encoding the Z4 22 kD zein protein, the Z10 promoter from a gene encoding a 10 kD zein protein, a Z27 promoter from a gene encoding a 27 kD zein protein, the A20 promoter from the gene encoding a 19 kD -zein protein, inducible promoters, such as the light inducible promoter derived from the pea rbcS gene and the actin promoter from rice, e.g., the actin 2 promoter (WO 00/70067); seed specific promoters, such as the phaseolin promoter from beans, may also be used. The nucleotide sequences of this invention can also be expressed under the regulation of promoters that are chemically regulated. This enables the nucleic acid sequence or encoded polypeptide to be synthesized only when the crop plants are treated with the inducing chemicals. Chemical induction of gene expression is detailed in EP 0 332 104 (to Ciba-Geigy) and U.S. Pat. No. 5,614,395. A useful promoter for chemical induction is the tobacco PR-1a promoter.

Examples of some constitutive promoters which have been described include the rice actin 1 (Wang et al., 1992; U.S. Pat. No. 5,641,876), CaMV 35S (Odell et al., 1985), CaMV 19S (Lawton et al., 1987), nos, Adh, sucrose synthase; and the ubiquitin promoters.

Examples of tissue specific promoters which have been described include the lectin (Vodkin, 1983; Lindstrom et al., 1990) corn alcohol dehydrogenase 1 (Vogel et al., 1992; Dennis et al., 1984), corn light harvesting complex (Simpson, 1986; Bansal et al., 1992), corn heat shock protein (Odell et al., 1985), pea small subunit RuBP carboxylase (Poulsen et al., 1986), Ti plasmid mannopine synthase (Langridge et al., 1989), Ti plasmid nopaline synthase (Langridge et al., 1989), petunia chalcone isomerase (vanTunen et al., 1988), bean glycine rich protein 1 (Keller et al., 1989), truncated CaMV 35s (Odell et al., 1985), potato patatin (Wenzler et al., 1989), root cell (Yamamoto et al., 1990), maize zein (Reina et al., 1990; Kriz et al., 1987; Wandelt et al., 1989; Langridge et al., 1983; Reina et al., 1990), globulin-1 (Belanger et al., 1991), α-tubulin, cab (Sullivan et al., 1989), PEPCase (Hudspeth & Grula, 1989), R gene complex-associated promoters (Chandler et al., 1989), histone, and chalcone synthase promoters (Franken et al., 1991). Tissue specific enhancers are described in Fromm et al. (1990).

Inducible promoters that have been described include the ABA- and turgor-inducible promoters, the promoter of the auxin-binding protein gene (Schwob et al., 1993), the UDP glucose flavonoid glycosyl-transferase gene promoter (Ralston et al., 1988), the MPI proteinase inhibitor promoter (Cordero et al., 1994), and the glyceraldehyde-3-phosphate dehydrogenase gene promoter (Kohler et al., 1995; Quigley et al., 1989; Martinez et al., 1989).

Several other tissue-specific regulated genes and/or promoters have been reported in plants. These include genes encoding the seed storage proteins (such as napin, cruciferin, beta-conglycinin, and phaseolin) zein or oil body proteins (such as oleosin), or genes involved in fatty acid biosynthesis (including acyl carrier protein, stearoyl-ACP desaturase. And fatty acid desaturases (fad 2-1)), and other genes expressed during embryo development (such as Bce4, see, for example, EP 255378 and Kridl et al., 1991). Particularly useful for seed-specific expression is the pea vicilin promoter (Czako et al., 1992). (See also U.S. Pat. No. 5,625,136, herein incorporated by reference.) Other useful promoters for expression in mature leaves are those that are switched on at the onset of senescence, such as the SAG promoter from Arabidopsis (Gan et al., 1995).

A class of fruit-specific promoters expressed at or during antithesis through fruit development, at least until the beginning of ripening, is discussed in U.S. Pat. No. 4,943,674. cDNA clones that are preferentially expressed in cotton fiber have been isolated (John et al., 1992). cDNA clones from tomato displaying differential expression during fruit development have been isolated and characterized (Mansson et al., 1985, Slater et al., 1985). The promoter for polygalacturonase gene is active in fruit ripening. The polygalacturonase gene is described in U.S. Pat. No. 4,535,060, U.S. Pat. No. 4,769,061, U.S. Pat. No. 4,801,590, and U.S. Pat. No. 5,107,065, which disclosures are incorporated herein by reference.

Other examples of tissue-specific promoters include those that direct expression in leaf cells following damage to the leaf (for example, from chewing insects), in tubers (for example, patatin gene promoter), and in fiber cells (an example of a developmentally-regulated fiber cell protein is E6 (John et al., 1992). The E6 gene is most active in fiber, although low levels of transcripts are found in leaf, ovule and flower.

The tissue-specificity of some “tissue-specific” promoters may not be absolute and may be tested by one skilled in the art using the diphtheria toxin sequence. One can also achieve tissue-specific expression with “leaky” expression by a combination of different tissue-specific promoters (Beals et al., 1997). Other tissue-specific promoters can be isolated by one skilled in the art (see U.S. Pat. No. 5,589,379). Several inducible promoters (“gene switches”) have been reported. Many are described in the review by Gatz (1996) and Gatz (1997). These include tetracycline repressor system, Lac repressor system, copper-inducible systems, salicylate-inducible systems (such as the PR1a system), glucocorticoid-(Aoyama et al., 1997) and ecdysome-inducible systems. Also included are the benzene sulphonamide-(U.S. Pat. No. 5,364,780) and alcohol-(WO 97/06269 and WO 97/06268) inducible systems and glutathione S-transferase promoters. Other studies have focused on genes inducibly regulated in response to environmental stress or stimuli such as increased salinity. Drought, pathogen and wounding. (Graham et al., 1985; Graham et al., 1985, Smith et al., 1986). Accumulation of metallocarboxypeptidase-inhibitor protein has been reported in leaves of wounded potato plants (Graham et al., 1981). Other plant genes have been reported to be induced methyl jasmonate, elicitors, heat-shock, anaerobic stress, or herbicide safeners.

Regulated expression of the chimeric transacting viral replication protein can be further regulated by other genetic strategies. For example, Cre-mediated gene activation as described by Odell et al. 1985. Thus, a DNA fragment containing 3′ regulatory sequence bound by lox sites between the promoter and the replication protein coding sequence that blocks the expression of a chimeric replication gene from the promoter can be removed by Cre-mediated excision and result in the expression of the trans-acting replication gene. In this case, the chimeric Cre gene, the chimeric trans-acting replication gene, or both can be under the control of tissue- and developmental-specific or inducible promoters. An alternate genetic strategy is the use of tRNA suppressor gene. For example, the regulated expression of a tRNA suppressor gene can conditionally control expression of a trans-acting replication protein coding sequence containing an appropriate termination codon as described by Ulmasov et al. 1997. Again, either the chimeric tRNA suppressor gene, the chimeric transacting replication gene, or both can be under the control of tissue- and developmental-specific or inducible promoters.

Frequently it is desirable to have continuous or inducible expression of a DNA sequence throughout the cells of an organism in a tissue-independent manner. For example, increased resistance of a plant to infection by soil- and airborne-pathogens might be accomplished by genetic manipulation of the plant's genome to comprise a continuous promoter operably linked to a heterologous pathogen-resistance gene such that pathogen-resistance proteins are continuously expressed throughout the plant's tissues.

Alternatively, it might be desirable to inhibit expression of a native DNA sequence within a plant's tissues to achieve a desired phenotype. In this case, such inhibition might be accomplished with transformation of the plant to comprise a constitutive, tissue-independent promoter operably linked to an antisense nucleotide sequence, such that constitutive expression of the antisense sequence produces an RNA transcript that interferes with translation of the mRNA of the native DNA sequence.

To define a minimal promoter region, a DNA segment representing the promoter region is removed from the 5′ region of the gene of interest and operably linked to the coding sequence of a marker (reporter) gene by recombinant DNA techniques well known to the art. The reporter gene is operably linked downstream of the promoter, so that transcripts initiating at the promoter proceed through the reporter gene. Reporter genes generally encode proteins which are easily measured, including, but not limited to, chloramphenicol acetyl transferase (CAT), beta-glucuronidase (GUS), green fluorescent protein (GFP), beta-galactosidase (beta-GAL), and luciferase.

The construct containing the reporter gene under the control of the promoter is then introduced into an appropriate cell type by transfection techniques well known to the art. To assay for the reporter protein, cell lysates are prepared and appropriate assays, which are well known in the art, for the reporter protein are performed. For example, if CAT were the reporter gene of choice, the lysates from cells transfected with constructs containing CAT under the control of a promoter under study are mixed with isotopically labeled chloramphenicol and acetyl-coenzyme A (acetyl-CoA). The CAT enzyme transfers the acetyl group from acetyl-CoA to the 2- or 3-position of chloramphenicol. The reaction is monitored by thin-layer chromatography, which separates acetylated chloramphenicol from unreacted material. The reaction products are then visualized by autoradiography.

The level of enzyme activity corresponds to the amount of enzyme that was made, which in turn reveals the level of expression from the promoter of interest. This level of expression can be compared to other promoters to determine the relative strength of the promoter under study. In order to be sure that the level of expression is determined by the promoter, rather than by the stability of the mRNA, the level of the reporter mRNA can be measured directly, such as by Northern blot analysis.

Once activity is detected, mutational and/or deletional analyses may be employed to determine the minimal region and/or sequences required to initiate transcription. Thus, sequences can be deleted at the 5′ end of the promoter region and/or at the 3′ end of the promoter region, and nucleotide substitutions introduced. These constructs are then introduced to cells and their activity determined.

In one embodiment of the invention, the promoter may be a gamma zein promoter, an oleosin ole16 promoter, a globulinI promoter, an actin I promoter, an actin c1 promoter, a sucrose synthetase promoter, an INOPS promoter, an EXM5 promoter, a globulin2 promoter, a b-32, ADPG-pyrophosphorylase promoter, an Ltp1 promoter, an Ltp2 promoter, an oleosin ole17 promoter, an oleosin ole18 promoter, an actin 2 promoter, a pollen-specific protein promoter, a pollen-specific pectate lyase promoter, an anther-specific protein promoter (Huffman), an anther-specific gene RTS2 promoter, a pollen-specific gene promoter, a tapeturn-specific gene promoter, tapeturn-specific gene RAB24 promoter, a anthranilate synthase alpha subunit promoter, an alpha zein promoter, an anthranilate synthase beta subunit promoter, a dihydrodipicolinate synthase promoter, a Thi1 promoter, an alcohol dehydrogenase promoter, a cab binding protein promoter, an H3C4 promoter, a RUBISCO SS starch branching enzyme promoter, an ACCase promoter, an actin3 promoter, an actin7 promoter, a regulatory protein GF14-12 promoter, a ribosomal protein L9 promoter, a cellulose biosynthetic enzyme promoter, an S-adenosyl-L-homocysteine hydrolase promoter, a superoxide dismutase promoter, a C-kinase receptor promoter, a phosphoglycerate mutase promoter, a root-specific RCc3 mRNA promoter, a glucose-6 phosphate isomerase promoter, a pyrophosphate-fructose 6-phosphatelphosphotransferase promoter, an ubiquitin promoter, a beta-ketoacyl-ACP synthase promoter, a 33 kDa photosystem 11 promoter, an oxygen evolving protein promoter, a 69 kDa vacuolar ATPase subunit promoter, a metallothionein-like protein promoter, a glyceraldehyde-3-phosphate dehydrogenase promoter, an ABA- and ripening-inducible-like protein promoter, a phenylalanine ammonia lyase promoter, an adenosine triphosphatase S-adenosyl-L-homocysteine hydrolase promoter, an a-tubulin promoter, a cab promoter, a PEPCase promoter, an R gene promoter, a lectin promoter, a light harvesting complex promoter, a heat shock protein promoter, a chalcone synthase promoter, a zein promoter, a globulin-1 promoter, an ABA promoter, an auxin-binding protein promoter, a UDP glucose flavonoid glycosyl-transferase gene promoter, an NTI promoter, an actin promoter, an opaque 2 promoter, a b70 promoter, an oleosin promoter, a CaMV 35S promoter, a CaMV 19S promoter, a histone promoter, a turgor-inducible promoter, a pea small subunit RuBP carboxylase promoter, a Ti plasmid mannopine synthase promoter, Ti plasmid nopaline synthase promoter, a petunia chalcone isomerase promoter, a bean glycine rich protein I promoter, a CaMV 35S transcript promoter, a potato patatin promoter, or a S-E9 small subunit RuBP carboxylase promoter.

In addition to promoters, a variety of 5′ and 3′ transcriptional regulatory sequences are also available for use in the present invention. Transcriptional terminators are responsible for the termination of transcription and correct mRNA polyadenylation. The 3′ nontranslated regulatory DNA sequence may include from about 50 to about 1,000, or about 100 to about 1,000, nucleotide base pairs and contains plant transcriptional and translational termination sequences. Appropriate transcriptional terminators and those which are known to function in plants include the CaMV 35S terminator, the tml terminator, the nopaline synthase terminator, the pea rbcS E9 terminator, the terminator for the T7 transcript from the octopine synthase gene of Agrobacterium tumefaciens, and the 3′ end of the protease inhibitor I or II genes from potato or tomato, although other 3′ elements known to those of skill in the art can also be employed. Alternatively, one also could use a gamma coixin, oleosin 3 or other terminator from the genus Coix.

Useful 3′ elements include those from the nopaline synthase gene of Agrobacterium tumefaciens (Bevan et al., 1983), the terminator for the T7 transcript from the octopine synthase gene of Agrobacterium tumefaciens, and the 3′ end of the protease inhibitor I or II genes from potato or tomato.

Other sequences that have been found to enhance gene expression in transgenic plants include intron sequences (e.g., from Adh1, bronze1, actin1, actin 2 (WO 00/760067), or the sucrose synthase intron) and viral leader sequences (e.g., from TMV, MCMV and AMV). For example, a number of non-translated leader sequences derived from viruses are known to enhance expression. Specifically, leader sequences from Tobacco Mosaic Virus (TMV), Maize Chlorotic Mottle Virus (MCMV), and Alfalfa Mosaic Virus (AMV) have been shown to be effective in enhancing expression (e.g., Gallie et al., 1987; Skuzeski et al., 1990). Other leaders known in the art include but are not limited to: Picornavirus leaders, for example, EMCV leader (Encephalomyocarditis 5 noncoding region) (Elroy-Stein et al., 1989); Potyvirus leaders, for example, TEV leader (Tobacco Etch Virus); MDMV leader (Maize Dwarf Mosaic Virus); Human immunoglobulin heavy-chain binding protein (BiP) leader, (Macejak et al., 1991); Untranslated leader from the coat protein mRNA of alfalfa mosaic virus (AMV RNA 4), (Jobling et al., 1987; Tobacco mosaic virus leader (TMV), (Gallie et al., 1989; and Maize Chlorotic Mottle Virus leader (MCMV) (Lommel et al., 1991. See also, Della-Cioppa et al., 1987.

As the DNA sequence between the transcription initiation site and the start of the coding sequence, i.e., the untranslated leader sequence, can influence gene expression, one may also wish to employ a particular leader sequence. Preferred leader sequences are contemplated to include those that include sequences predicted to direct optimum expression of the attached gene, i.e., to include a preferred consensus leader sequence that may increase or maintain mRNA stability and prevent inappropriate initiation of translation. The choice of such sequences will be known to those of skill in the art in light of the present disclosure. Sequences that are derived from genes that are highly expressed in plants will be most useful.

Regulatory elements such as Adh intron 1 (Callis et al., 1987), sucrose synthase intron (Vasil et al., 1989) or TMV omega element (Gallie, et al., 1989), may further be included where desired.

Examples of enhancers include elements from the CaMV 35S promoter, octopine synthase genes (Ellis el al., 1987), the rice actin I gene, the maize alcohol dehydrogenase gene (Callis et al., 1987), the maize shrunken I gene (Vasil et al., 1989), TMV Omega element (Gallie et al., 1989) and promoters from non-plant eukaryotes (e.g. yeast; Ma et al., 1988).

Vectors for use in accordance with the present invention may be constructed to include the octopine synthase (ocs) enhancer element. This element was first identified as a 16 bp palindromic enhancer from the ocs gene of ultilane (Ellis et al., 1987), and is present in at least 10 other promoters (Bouchez et al., 1989). The use of an enhancer element, such as the ocs element and particularly multiple copies of the element, will act to increase the level of transcription from adjacent promoters when applied in the context of monocot transformation.

Vectors for use in tissue-specific targeting of genes in transgenic plants will typically include tissue-specific promoters and may also include other tissue-specific control elements such as enhancer sequences. Promoters which direct specific or enhanced expression in certain plant tissues will be known to those of skill in the art in light of the present disclosure. These include, for example, the rbcS promoter, specific for green tissue; the ocs, nos and mas promoters which have higher activity in roots or wounded leaf tissue; a truncated (−90 to +8) 35S promoter which directs enhanced expression in roots, an alpha-tubulin gene that directs expression in roots and promoters derived from zein storage protein genes which direct expression in endosperm. It is particularly contemplated that one may advantageously use the 16 bp ocs enhancer element from the octopine synthase (ocs) gene (Ellis et al., 1987; Bouchez et al., 1989), especially when present in multiple copies, to achieve enhanced expression in roots.

Tissue specific expression may be functionally accomplished by introducing a constitutively expressed gene construct (all tissues) in combination with a transcription factor of the present invention that represses transcription of the introduced construct and that is expressed only in those tissues where the gene product is not desired. Alternatively, tissue specific expression can be achieved by introducing a first construct expression of which is normally repressed in combination with a second construct containing a transcription factor of the present invention which induces expression of the first construct operably linked to a tissue specific promoter.

Expression of some genes in transgenic plants will be desired only under specified conditions. For example, it is proposed that expression of certain transcription factors of the present invention that confer resistance to environmental stress factors such as drought will be desired only under actual stress conditions. It is contemplated that expression of such genes throughout a plants development may have detrimental effects. It is known that a large number of genes exist that respond to the environment. For example, expression of some genes such as rbcS, encoding the small subunit of ribulose bisphosphate carboxylase, is regulated by light as mediated through phytochrome. Other genes are induced by secondary stimuli. For example, synthesis of abscisic acid (ABA) is induced by certain environmental factors, including but not limited to water stress. A number of genes have been shown to be induced by ABA (Skriver and Mundy, 1990). It is also anticipated that expression of transcription factor genes conferring resistance to insect predation would be desired only under conditions of actual insect infestation. Therefore, for some desired traits inducible expression of genes in transgenic plants will be desired.

Expression of a gene in a transgenic plant may be desired only in a certain time period during the development of the plant. Developmental timing is frequently correlated with tissue specific gene expression. For example, expression of zein storage proteins is initiated in the endosperm about 15 days after pollination.

For quantification and determination of localization of expression within a plant, i.e., a tissue, a number of tools are suited. Detection systems can readily be created or are available which are based on, e.g., immunochemical, enzymatic, fluorescent detection and quantification. Protein levels can be determined in plant tissue extracts or in intact tissue using in situ analysis of protein expression.

Generally, individual transformed lines with one chimeric promoter reporter construct will vary in their levels of expression of an included reporter gene. Also frequently observed is the phenomenon that such transformants do not express any detectable product (RNA or protein). The variability in expression is commonly ascribed to ‘position effects’, although the molecular mechanisms underlying this inactivity are usually not clear.

### POLYNUCLEOTIDES, PROTEINS AND VECTORS OF THE INVENTION

The invention encompasses isolated or substantially purified nucleic acid or protein compositions. In the context of the present invention, an “isolated” or “purified” DNA molecule or an “isolated” or “purified” polypeptide is a DNA molecule or polypeptide that, by the hand of man, exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized.

An “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein of interest chemicals.

The nucleotide sequences of the invention include both the naturally occurring sequences as well as mutant (variant) forns. Such variants will continue to possess the desired activity, i.e., either transcription factor activity or the activity of the product encoded by the open reading frame of the non-variant nucleotide sequence.

Thus, by “variants” is intended substantially similar sequences. For nucleotide sequences comprising an open reading frame, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis and for open reading frames, encode the native protein, as well as those that encode a polypeptide having amino acid substitutions relative to the native protein. Generally, nucleotide sequence variants of the invention will have at least 40, 50, 60, to 70%, at least 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98% and 99% nucleotide sequence identity to the native (wild type or endogenous) nucleotide sequence.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence, a gap penalty is typically introduced and is subtracted from the number of matches.

Methods of alignment of sequences for comparison are well known in the art. Thus, the determination of percent identity between any two sequences can be accomplished using a mathematical algorithm. Preferred, non-limiting examples of such mathematical algorithms are the algorithm of Myers and Miller, 1988; the local homology algorithm of Smith et al. 1981; the homology alignment algorithm of Needleman and Wunsch 1970; the search-for-similarity-method of Pearson and Lipman 1988; and the algorithm of Karlin and Altschul, 1990, modified as in Karlin and Altschul, 1993.

In one embodiment, optimal alignment is conducted using the homology alignment algorithm of Needleman and Wunsch (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. 1988; Higgins et al. 1989; Corpet et al. 1988; Huang et al. 1992; and Pearson et al. 1994. The ALIGN program is based on the algorithm of Myers and Miller, supra. The BLAST programs of Altschul et al., 1990, are based on the algorithm of Karlin and Altschul supra.

Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences (see, e.g., Karlin & Altschul, 1993). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, or less than about 0.01, or less than about 0.001.

To obtain gapped alignments for comparison purposes, Gapped BLAST (in BLAST 2.0) can be utilized as described in Altschul et al. 1997. Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g. BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, 1989). See http://www.ncbi.nlm.nih.gov. Alignment may also be performed manually by inspection.

For purposes of the present invention, comparison of nucleotide sequences for determination of percent sequence identity to the promoter sequences disclosed herein is preferably made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

“Significant increase” is an increase that is larger than the margin of error inherent in the measurement technique, preferably an increase by about 2-fold or greater.

“Significantly less” means that the decrease is larger than the margin of error inherent in the measurement technique, preferably a decrease by about 2-fold or greater.

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e) The term “substantial identity” of polynucleotide sequences means that a polynucleotide comprises a sequence that has at least 70%, 71%, 72%, 73%, 74%, 75%, 76%, 77%, 78%, or 79%, at least 80%, 81%, 82%, 83%, 84%, 85%, 86%, 87%, 88%, or 89%, at least 90%, 91%, 92%, 93%, or 94%, and at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, at least 90%, and at least 95%.

Another indication that nucleotide sequences are substantially identical is if two molecules hybridize to each other under moderate or stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein.

The phrase “hybridize to” or “specifically hybridizing to” refers to the binding, duplexing, or hybridizing of a molecule only to a particular nucleotide sequence under moderate to stringent conditions, with respect to specificity, when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridization are sequence dependent, and are different under different environmental parameters. The Tₘ is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl, 1984; Tₘ 81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity.

For example, if sequences with >90% identity are sought, the Tₘ can be decreased 10° C. Generally, stringent conditions are selected to be about 5° C. lower than the thermal melting point for the specific sequence and its complement at a defined ionic strength and pH. However, severely stringent conditions can utilize a hybridization and/or wash at 1, 2, 3, or 4° C. lower than the thermal melting point; moderately stringent conditions can utilize a hybridization and/or wash at 6, 7, 8, 9, or 10° C. lower than the thermal melting point; low stringency conditions can utilize a hybridization and/or wash at 11, 12, 13, 14, 15, or 20° C. lower than the thermal melting point. Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. An extensive guide to the hybridization of nucleic acids is found in Tijssen, 1993. Generally, highly stringent hybridization and wash conditions are selected to be about 5° C. lower than the thermal melting point Tₘ for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more typically about 0.01 to 1.0 M, Na ion concentration (or other salts) at pH 7.0 to 8.3, and the temperature is typically at least about 30° C. and at least about 60° C. for long robes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

The following are examples of sets of hybridization/wash conditions that may be used to clone orthologous nucleotide sequences that are substantially identical to reference nucleotide sequences of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or from human manipulation. Methods for such manipulations are generally known in the art.

Thus, the polypeptides may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. See, for example, Kunkel, 1985; Kunkel et al., 1987; U.S. Pat. No. 4,873,192; Walker and Gaastra, 1983 and the references cited therein. Guidance as to appropriate amino acid substitutions that do not affect biological activity of the protein of interest may be found in the model of Dayhoff et al. (1978). Conservative substitutions, such as exchanging one amino acid with another having similar properties, are preferred.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine I, Lysine (K), Histidine (H); Acidic: Aspartic acid (D), Glutamic acid (E), Asparagine (N), Glutamine (Q). See also, Creighton, 1984. In addition, individual substitutions, deletions or additions which alter, add or delete a single amino acid or a small percentage of amino acids in an encoded sequence are also “conservatively modified variations.”

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in each described sequence.

The nucleic acid molecules of the invention can be “optimized” for enhanced expression in plants of interest. See, for example, EPA 035472; WO 91/16432; Perlak et al., 1991; and Murray et al., 1989. In this manner, the open reading frames in genes or gene fragments can be synthesized utilizing plant-preferred codons. See, for example, Campbell and Gowri (1990), for a discussion of host-preferred codon usage. Thus, the nucleotide sequences can be optimized for expression in any plant. It is recognized that all or any part of the gene sequence may be optimized or synthetic. That is, synthetic or partially optimized sequences may also be used. Variant nucleotide sequences and proteins also encompass sequences and protein derived from a mutagenic and recombinogenic procedure such as DNA shuffling. With such a procedure, one or more different coding sequences can be manipulated to create a new polypeptide possessing the desired properties. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art. See, for example, Stemmer, 1994; Stemmer, 1994; Crameri et al., 1997; Moore et al., 1997; Zhang et al., 1997; Crameri et al., 1998; and U.S. Pat. Nos. 5,605,793 and 5,837,458.

DNA useful for introduction into plant cells includes that which has been derived or isolated from any source, that may be subsequently characterized as to structure, size and/or function, chemically altered, and later introduced into plants. An example of DNA “derived” from a source, would be a DNA sequence that is identified as a useful fragment within a given organism, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering. Such DNA is commonly referred to as “recombinant DNA.”

Therefore useful DNA includes completely synthetic DNA, semi-synthetic DNA, DNA isolated from biological sources, and DNA derived from introduced RNA. Generally, the introduced DNA is not originally resident in the plant genotype which is the recipient of the DNA, but it is within the scope of the invention to isolate a gene from a given plant genotype, and to subsequently introduce multiple copies of the gene into the same genotype, e.g., to enhance production of a given gene product such as a storage protein or a protein that confers tolerance or resistance to a biotic or abiotic stress.

The introduced DNA includes but is not limited to, DNA from plant genes such as the transcription factors described herein, and non-plant genes such as those from bacteria, yeasts, animals or viruses. The introduced DNA can include modified genes, portions of genes, or chimeric genes, including genes from the same or different genotype.

The introduced DNA used for transformation herein may be circular or linear, double-stranded or single-stranded. Generally, the DNA is in the form of chimeric DNA, such as plasmid DNA, that can also contain coding regions flanked by regulatory sequences which promote the expression of the recombinant DNA present in the resultant plant. For example, the DNA may itself comprise or consist of a promoter that is active in a plant which is derived from a source other than that plant, or may utilize a promoter already present in a plant genotype that is the transformation target.

Generally, the introduced DNA will be relatively small, i.e., less than about 30 kb to minimize any susceptibility to physical, chemical, or enzymatic degradation which is known to increase as the size of the DNA increases. As noted above, the number of proteins, RNA transcripts or mixtures thereof which is introduced into the plant genome is preferably preselected and defined, e.g., from one to about 5-10 such products of the introduced DNA may be formed.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one which is naturally occurring but has been obtained in a recombinant form useful for heterologous expression. The expression of the nucleotide sequence in the expression cassette may be under the control of a constitutive promoter or of an inducible promoter which initiates transcription only when the host cell is exposed to some particular external stimulus. In the case of a multicellular organism, the promoter can also be specific to a particular tissue or organ or stage of development.

“Vector” is defined to include, inter alia, any plasmid, cosmid, phage or Agrobacterium binary vector in double or single stranded linear or circular form which may or may not be self transmissible or mobilizable, and which can transform prokaryotic or eukaryotic host either by integration into the cellular genome or exist extrachromosomally (e.g. autonomous replicating plasmid with an origin of replication).

Specifically included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells).

Preferably the nucleic acid in the vector is under the control of, and operably linked to, an appropriate promoter or other regulatory elements for transcription in a host cell such as a microbial, e.g. bacterial, or plant cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell.

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

“Recombinant DNA molecule” is a combination of DNA sequences that are joined together using recombinant DNA technology and procedures used to join together DNA sequences as described, for example, in Sambrook et al., 1989.

“DNA shuffling” is a method to introduce mutations or rearrangements, preferably randomly, in a DNA molecule or to generate exchanges of DNA sequences between two or more DNA molecules, preferably randomly. The DNA molecule resulting from DNA shuffling is a shuffled DNA molecule that is a non-naturally occurring DNA molecule derived from at least one template DNA molecule. The shuffled DNA preferably encodes a variant polypeptide modified with respect to the polypeptide encoded by the template DNA, and may have an altered biological activity with respect to the polypeptide encoded by the template DNA.

The word “plant” refers to any plant, particularly to seed plant, and “plant cell” is a structural and physiological unit of the plant, which comprises a cell wall but may also refer to a protoplast. The plant cell may be in form of an isolated single cell or a cultured cell, or as a part of higher organized unit such as, for example, a plant tissue, or a plant organ.

A “transgenic plant” is a plant having one or more plant cells that contain an expression vector.

The term “altered plant trait” means any phenotypic or genotypic change in a transgenic plant relative to the wild-type or non-transgenic plant host.

“Plant tissue” includes differentiated and undifferentiated tissues or plants, including but not limited to roots, stems, shoots, leaves, pollen, seeds, tumor tissue and various forms of cells and culture such as single cells, protoplast, embryos, and callus tissue. The plant tissue may be in plants or in organ, tissue or cell culture. In part the invention relates to an isolated plant, e.g., rice, nucleic acid molecule comprising a gene having an open reading frame which encodes a transcription factor and in particular transcription factors encoded by any one of SEQ ID NO.: 1-70, as well as the endogenous plant promoters for those genes. The nucleic acid molecules can be used by overexpressing nucleic acid molecules, or by altering the expression of host genes, e.g., by “knocking out” the expression of at least one genomic copy of the gene. Plants having genetic disruptions in host genes may be less susceptible to infection or stress, e.g., due to a decrease or absence of a host protein needed for infection or stress related response, or, alternatively, hypersusceptible to infection or stress. Plants that are hypersusceptible to infection or stress may be useful to prepare transgenic plants as the expression of the gene(s) which was disrupted may be related to gene silencing.

Virtually any DNA composition may be used for delivery to recipient plant cells, e.g., monocotyledonous cells, to ultimately produce fertile transgenic plants in accordance with the present invention. For example, DNA segments in the form of vectors and plasmids, or linear DNA fragments, in some instances containing only the DNA element to be expressed in the plant, and the like, may be employed. The construction of vectors which may be employed in conjunction with the present invention will be known to those of skill of the art in light of the present disclosure (see, e.g., Sambrook et al., 1989; Gelvin et al., 1990).

Vectors, plasmids, cosmids, YACs (yeast artificial chromosomes), BACs (bacterial artificial chromosomes) and DNA segments for use in transforming such cells will, of course, generally comprise the cDNA, gene or genes which one desires to introduce into the cells. These DNA constructs can further include structures such as promoters, enhancers, polylinkers, or even regulatory genes as desired. The DNA segment or gene chosen for cellular introduction will often encode a protein which will be expressed in the resultant recombinant cells, such as will result in a screenable or selectable trait and/or which will impart an improved phenotype to the regenerated plant. However, this may not always be the case, and the present invention also encompasses transgenic plants incorporating non-expressed transgenes.

In certain embodiments, it is contemplated that one may wish to employ replication-competent viral vectors in monocot transformation. Such vectors include, for example, wheat dwarf virus (WDV) “shuttle” vectors, such as pW1-11 and PW1-GUS (Ugaki et al., 1991). These vectors are capable of autonomous replication in maize cells as well as E. coli, and as such may provide increased sensitivity for detecting DNA delivered to transgenic cells. A replicating vector may also be useful for delivery of genes flanked by DNA sequences from transposable elements such as Ac, Ds, or Mu. It has been proposed (Laufs et al., 1990) that transposition of these elements within the maize genome requires DNA replication. It is also contemplated that transposable elements would be useful for introducing DNA fragments lacking elements necessary for selection and maintenance of the plasmid vector in bacteria, e.g., antibiotic resistance genes and origins of DNA replication. It is also proposed that use of a transposable element such as Ac, Ds, or Mu would actively promote integration of the desired DNA and hence increase the frequency of stably transformed cells. The use of a transposable element such as Ac, Ds, or Mu may actively promote integration of the DNA of interest and hence increase the frequency of stably transformed cells. Transposable elements may be useful to allow separation of genes of interest from elements necessary for selection and maintenance of a plasmid vector in bacteria or selection of a transformant. By use of a transposable element, desirable and undesirable DNA sequences may be transposed apart from each other in the genome, such that through genetic segregation in progeny, one may identify plants with either the desirable or the undesirable DNA sequences.

Additionally, vectors may be constructed and employed in the intracellular targeting of a specific transcription factor within the cells of a transgenic plant or in directing a protein to the extracellular environment. This will generally be achieved by joining a DNA sequence encoding a transit or signal peptide sequence to the coding sequence of a particular transcription factor gene. The resultant transit, or signal, peptide will transport the protein to a particular intracellular, or extracellular destination, respectively, and will then be post-translationally removed. Transit or signal peptides act by facilitating the transport of proteins through intracellular membranes, e.g., vacuole, vesicle, plastid and mitochondrial membranes, whereas signal peptides direct proteins through the extracellular membrane.

A particular example of such a use concerns the direction of a herbicide resistance gene, such as the EPSPS gene, to a particular organelle such as the chloroplast rather than to the cytoplasm. This is exemplified by the use of the rbcs transit peptide which confers plastid-specific targeting of proteins. In addition, it is proposed that it may be desirable to target certain genes responsible for male sterility to the mitochondria, or to target certain genes for resistance to phytopathogenic organisms to the extracellular spaces, or to target proteins to the vacuole.

By facilitating the transport of the protein into compartments inside and outside the cell, these sequences may increase the accumulation of gene product protecting them from proteolytic degradation. These sequences also allow for additional mRNA sequences from highly expressed genes to be attached to the coding sequence of the genes. Since mRNA well suited for the isolation of gene sequences from any source organism, preferably other plant species. In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as p, or any other detectable marker. Thus, for example, probes for hybridization can be made by labeling synthetic oligonucleotides based on the sequence of the invention. Methods for preparation of probes for hybridization and for construction of cDNA and genomic libraries are generally known in the art and are disclosed in Sambrook et al. (1989). In general, sequences that hybridize to the sequences disclosed herein will have at least 40% to 50%, about 60% to 70% and even about 80% 85%, 90%, 95% to 98% or more identity with the disclosed sequences. That is, the sequence similarity of sequences may range, sharing at least about 40% to 50%, about 60% to 70%, and even about 80%, 85%, 90%, 95% to 98% sequence similarity.

The nucleic acid molecules of the invention can also be used to identify other orthologs for example, by a search of known databases for genes encoding polypeptides having a specified amino acid sequence identity or DNA having a specified nucleotide sequence identity. Methods of alignment of sequences for comparison are well known in the art and are described hereinabove.

It is specifically contemplated by the inventors that one could mutagenize DNA having an open reading frame to, for example, potentially improve the utility of the DNA for expression of transgenes in plants. The mutagenesis can be carried out at random and the mutagenized sequences screened for activity in a trial-by-error procedure. Alternatively, particular sequences which provide the promoter with desirable expression characteristics, or a promoter with expression enhancement activity, could be identified and these or similar sequences introduced into the sequences via mutation. It is further contemplated that one being translated by ribosomes is more stable than naked mRNA, the presence of translatable mRNA in front of the gene may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. Targeting of certain proteins may be desirable in order to enhance the stability of the protein (U.S. Pat. No. 5,545,818).

It may be useful to target DNA itself within a cell. For example, it may be useful to target introduced DNA to the nucleus as this may increase the frequency of transformation. Within the nucleus itself it would be useful to target a gene in order to achieve site specific integration. For example, it would be useful to have an gene introduced through transformation replace an existing gene in the cell.

According to one embodiment, the present invention is directed to a nucleic acid molecule comprising a nucleotide sequence isolated or obtained from any plant which encodes a polypeptide having at least 70% amino acid sequence identity to a polypeptide encoded by a gene comprising any one of SEQ ID NO.: 1-70. Based on the nucleic acid sequences of the present invention, orthologs may be identified or isolated from the genome of any desired organism, preferably from another plant, according to well known techniques based on their sequence similarity to the rice nucleic acid sequences, e.g., hybridization, PCR or computer generated sequence comparisons.

All or a portion of the nucleic acid sequences disclosed herein can be used as a probe that selectively hybridizes to other gene sequences present in a population of cloned genomic DNA fragments or cDNA fragments (i.e., genomic or cDNA libraries) from a chosen source organism. Further, suitable genomic and cDNA libraries may be prepared from any cell or tissue of an organism. Such techniques include hybridization screening of plated DNA libraries (either plaques or colonies; see, e.g., Sambrook et al., 1989) and amplification by PCR using oligonucleotide primers preferably corresponding to sequence domains conserved among related polypeptide or subsequences of the nucleotide sequences provided herein (see, e.g., Innis et al., 1990). These methods are particularly well suited to the isolation of gene sequences from organisms closely related to the organism from which the probe sequence is derived. The application of these methods using the sequences disclosed herein as probes is which includes within its sequence a DNA sequence which encodes the promoter. An oligonucleotide primer bearing the desired mutated sequence is prepared, generally synthetically. This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation.

This heteroduplex vector is then used to transform or transfect appropriate cells, such as E. coli cells, and cells are selected which include recombinant vectors bearing the mutated sequence arrangement. Vector DNA can then be isolated from these cells and used for plant transformation. A genetic selection scheme was devised by Kunkel et al. (1987) to enrich for clones incorporating mutagenic oligonucleotides. Alternatively, the use of PCR with commercially available thermostable enzymes such as Taq polymerase may be used to incorporate a mutagenic oligonucleotide primer into an amplified DNA fragment that can then be cloned into an appropriate cloning or expression vector. The PCR-mediated mutagenesis procedures of Tomic et al. (1990) and Upender et al. (1995) provide two examples of such protocols. A PCR employing a thermostable ligase in addition to a thermostable polymerase also may be used to incorporate a phosphorylated mutagenic oligonucleotide into an amplified DNA fragment that may then be cloned into an appropriate cloning or expression vector. The mutagenesis procedure described by Michael (1994) provides an example of one such protocol.

The preparation of sequence variants of DNA segments using site-directed mutagenesis is provided as a means of producing potentially useful species and is not meant to be limiting as there are other ways in which sequence variants of DNA sequences may be obtained. For example, recombinant vectors encoding the desired promoter sequence may be treated with mutagenic agents, such as hydroxylamine, to obtain sequence variants.

In addition, an unmodified or modified nucleotide sequence of the present invention can be varied by shuffling the sequence of the invention. To test for a function of variant DNA sequences according to the invention, the sequence of interest is operably linked to a selectable or screenable marker gene and expression of the marker gene is tested in transient expression assays with protoplasts or in stably transformed plants. It is known to the skilled could mutagenize these sequences in order to enhance their expression of transgenes in a particular species.

The means for mutagenizing a DNA segment of the current invention are well-known to those of skill in the art. As indicated, modifications may be made by random or site-specific mutagenesis procedures. The DNA may be modified by altering its structure through the addition or deletion of one or more nucleotides from the sequence which encodes the corresponding unmodified sequences.

Mutagenesis may be performed in accordance with any of the techniques known in the art, such as, and not limited to, synthesizing an oligonucleotide having one or more mutations within the sequence of a particular regulatory region. In particular, site-specific mutagenesis is a technique useful in the preparation of mutants, through specific mutagenesis of the underlying DNA. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to about 75 nucleotides or more in length is preferred, with about 10 to about 25 or more residues on both sides of the junction of the sequence being altered.

In general, the technique of site-specific mutagenesis is well known in the art, as exemplified by various publications. As will be appreciated, the technique typically employs a phage vector which exists in both a single stranded and double stranded form. Typical vectors useful in site-directed mutagenesis include vectors such as the M13 phage. These phage are readily commercially available and their use is generally well known to those skilled in the art.

Double stranded plasmids also are routinely employed in site directed mutagenesis which eliminates the step of transferring the gene of interest from a plasmid to a phage.

In general, site-directed mutagenesis in accordance herewith is performed by first obtaining a single-stranded vector or melting apart of two strands of a double stranded vector artisan that DNA sequences capable of driving expression of an associated nucleotide sequence are build in a modular way. Accordingly, expression levels from shorter DNA fragments may be different than the one from the longest fragment and may be different from each other. For example, deletion of a down-regulating upstream element will lead to an increase in the expression levels of the associated nucleotide sequence while deletion of an up-regulating element will decrease the expression levels of the associated nucleotide sequence. It is also known to the skilled artisan that deletion of development-specific or a tissue-specific element will lead to a temporally or spatially altered expression profile of the associated nucleotide sequence.

As used herein, the term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” also is intended to refer to a process that involves the template-dependent extension of a primer molecule. The term template-dependent process refers to nucleic acid synthesis of an RNA or a DNA molecule wherein the sequence of the newly synthesized strand of nucleic acid is dictated by the well-known rules of complementary base pairing (see, for example, Watson and Rarnstad, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224. A number of template dependent processes are available to amplify the target sequences of interest present in a sample, such methods being well known in the art and specifically disclosed herein below.

Where a clone comprising a promoter has been isolated in accordance with the instant invention, one may wish to delimit the essential promoter regions within the clone. One efficient, targeted means for preparing mutagenizing promoters relies upon the identification of putative regulatory elements within the promoter sequence. This can be initiated by comparison with promoter sequences known to be expressed in similar tissue-specific or developmentally unique manner. Sequences which are shared among promoters with similar expression patterns are likely candidates for the binding of transcription factors and are thus likely elements which confer expression patterns. Confirmation of these putative regulatory elements can be achieved by deletion analysis of each putative regulatory region followed by functional analysis of each deletion construct by assay of a reporter gene which is functionally attached to each construct. As such, once a starting promoter sequence is provided, any of a number of different deletion mutants of the starting promoter could be readily prepared.

As indicated above deletion mutants of the promoter of the invention also could be randomly prepared and then assayed. With this strategy, a series of constructs are prepared, each containing a different portion of the clone (a subclone), and these constructs are then screened for activity. A suitable means for screening for activity is to attach a deleted promoter or intron construct which contains a deleted segment to a selectable or screenable marker, and to isolate only those cells expressing the marker gene. In this way, a number of different, deleted promoter constructs are identified which still retain the desired, or even enhanced, activity. The smallest segment which is required for activity is thereby identified through comparison of the selected constructs. This segment may then be used for the construction of vectors for the expression of exogenous genes.

In order to improve the ability to identify transformants, one may desire to employ a selectable or screenable marker gene as, or in addition to, the expressible gene of interest. “Marker genes” are genes that impart a distinct phenotype to cells expressing the marker gene and thus allow such transformed cells to be distinguished from cells that do not have the marker. Such genes may encode either a selectable or screenable marker, depending on whether the marker confers a trait which one can ‘select’ for by chemical means, i.e., through the use of a selective agent (e.g., a herbicide, antibiotic, or the like), or whether it is simply a trait that one can identify through observation or testing, i.e., by ‘screening’ (e.g., the R-locus trait, the green fluorescent protein (GFP)). Of course, many examples of suitable marker genes are known to the art and can be employed in the practice of the invention.

Included within the terms selectable or screenable marker genes are also genes which encode a “secretable marker” whose secretion can be detected as a means of identifying or selecting for transformed cells. Examples include markers which encode a secretable antigen that can be identified by antibody interaction, or even secretable enzymes which can be detected by their catalytic activity. Secretable proteins fall into a number of classes, including small, diffusible proteins detectable, e.g., by ELISA; small active enzymes detectable in extracellular solution (e.g., alpha-amylase, beta-lactamase, phosphinothricin acetyltransferase); and proteins that are inserted or trapped in the cell wall (e.g., proteins that include a leader sequence such as that found in the expression unit of extensin or tobacco PR-S).

With regard to selectable secretable markers, the use of a gene that encodes a protein that becomes sequestered in the cell wall, and which protein includes a unique epitope is considered to be particularly advantageous. Such a secreted antigen marker would ideally employ an epitope sequence that would provide low background in plant tissue, a promoter-leader sequence that would impart efficient expression and targeting across the plasma membrane, and would produce protein that is bound in the cell wall and yet accessible to antibodies. A normally secreted wall protein modified to include a unique epitope would satisfy all such requirements.

One example of a protein suitable for modification in this manner is extensin, or hydroxyproline rich glycoprotein (HPRG). For example, the maize HPRG (Steifel et al., 1990) molecule is well characterized in terms of molecular biology, expression and protein structure. However, any one of a variety of ultilane and/or glycine-rich wall proteins (Keller et al., 1989) could be modified by the addition of an antigenic site to create a screenable marker.

One exemplary embodiment of a secretable screenable marker concerns the use of a maize sequence encoding the wall protein HPRG, modified to include a 15 residue epitope from the pro-region of murine interleukin, however, virtually any detectable epitope may be employed in such embodiments, as selected from the extremely wide variety of antigen-antibody combinations known to those of skill in the art. The unique extracellular epitope can then be straightforwardly detected using antibody labeling in conjunction with chromogenic or fluorescent adjuncts.

Elements of the present disclosure may be exemplified in detail through the use of the bar and/or GUS genes, and also through the use of various other markers. Of course, in light of this disclosure, numerous other possible selectable and/or screenable marker genes will be apparent to those of skill in the art in addition to the one set forth hereinbelow. Therefore, it will be understood that the following discussion is exemplary rather than exhaustive. In light of the techniques disclosed herein and the general recombinant techniques which are known in the art, the present invention renders possible the introduction of any gene, including marker genes, into a recipient cell to generate a transformed plant.

Possible selectable markers for use in connection with the present invention include, but are not limited to, a neo gene which codes for kanamycin resistance and can be selected for using kanamycin, G418, paromomycin, and the like; a bar gene which codes for bialaphos or phosphinothricin resistance; a gene which encodes an altered EPSP synthase protein (Hinchee et al., 1988) thus conferring glyphosate resistance; a nitrilase gene such as bxn from Klebsiella ozaenae which confers resistance to bromoxynil (Stalker et al., 1988); a mutant acetolactate synthase gene (ALS) which confers resistance to imidazolinone, sulfonylurea or other ALS-inhibiting chemicals (European Patent Application 154,204, 1985); a methotrexate-resistant DHFR gene (Thillet et al., 1988); a dalapon dehalogenase gene that confers resistance to the herbicide dalapon; a mutated anthranilate synthase gene that confers resistance to 5-methyl tryptophan. Preferred selectable marker genes encode phosphinothricin acetyltransferase; glyphosate resistant EPSPS, aminoglycoside phosphotransferase; hygromycin phosphotransferase, or neomycin phosphotransferase. Where a mutant EPSP synthase gene is employed, additional benefit may be realized through the incorporation of a suitable chloroplast transit peptide, CTP (European Patent Application 0,218,571, 1987).

An illustrative embodiment of a selectable marker gene capable of being used in systems to select transformants is the genes that encode the enzyme phosphinothricin acetyltransferase, such as the bar gene from Streptomyces hygroscopicus or the pat gene from Streptomyces viridochromogenes. The enzyme phosphinothricin acetyl transferase (PAT) inactivates the active ingredient in the herbicide bialaphos, phosphinothricin (PPT). PPT inhibits glutamine synthetase, (Murakami et al., 1986; Twell et al., 1989) causing rapid accumulation of ammonia and cell death. The success in using this selective system in conjunction with monocots was particularly surprising because of the major difficulties which have been reported in transformation of cereals.

Where one desires to employ a bialaphos resistance gene in the practice of the invention, a particularly useful gene for this purpose is the bar or pat genes obtainable from species of Streptomyces (e.g., ATCC No. 21,705). The cloning of the bar gene has been described (Murakami et al., 1986; Thompson et al., 1987) as has the use of the bar gene in the context of plants other than monocots (De Block et al., 1987; De Block et al., 1989).

Selection markers resulting in positive selection, such as a phosphomannose isomerase gene, as described in patent application WO 93/05163, may also be used. Alternative genes to be used for positive selection are described in WO 94/20627 and encode xyloisomerases and phosphomanno-isomerases such as mannose-6-phosphate isomerase and mannose-1-phosphate isomerase; phosphomanno mutase; mannose epimerases such as those which convert carbohydrates to mannose or mannose to carbohydrates such as glucose or galactose; phosphatases such as mannose or xylose phosphatase, mannose-6-phosphatase and mannose-1-phosphatase, and permeases which are involved in the transport of mannose, or a derivative, or a precursor thereof into the cell. Transformed cells are identified without damaging or killing the non-transformed cells in the population and without co-introduction of antibiotic or herbicide resistance genes. As described in WO 93/05163, in addition to the fact that the need for antibiotic or herbicide resistance genes is eliminated, it has been shown that the positive selection method is often far more efficient than traditional negative selection.

Screenable markers that may be employed include, but are not limited to, a beta-glucuronidase (GUS) or uidA gene which encodes an enzyme for which various chromogenic substrates are known; an R-locus gene, which encodes a product that regulates the production of anthocyanin pigments (red color) in plant tissues (Dellaporta et al., 1988); a beta-lactamase gene (Sutcliffe, 1978), which encodes an enzyme for which various chromogenic substrates are known (e.g., PADAC, a chromogenic cephalosporin); a xylE gene (Zukowsky et al., 1983) which encodes a catechol dioxygenase that can convert chromogenic catechols; an α-amylase gene (Ikuta et al., 1990); a tyrosinase gene (Katz et al., 1983) which encodes an enzyme capable of oxidizing tyrosine to DOPA and dopaquinone which in turn condenses to form the easily detectable compound melanin; a β-galactosidase gene, which encodes an enzyme for which there are chromogenic substrates; a luciferase (lux) gene (Ow et al., 1986), which allows for bioluminescence detection; or even an aequorin gene (Prasher et al., 1985), which may be employed in calcium-sensitive bioluminescence detection, or a green fluorescent protein gene (Niedz et al., 1995).

Genes from the maize R gene complex are contemplated to be particularly useful as screenable markers. The R gene complex in maize encodes a protein that acts to regulate the production of anthocyanin pigments in most seed and plant tissue. A gene from the R gene complex was applied to maize transformation, because the expression of this gene in transformed cells does not harm the cells. Thus, an R gene introduced into such cells will cause the expression of a red pigment and, if stably incorporated, can be visually scored as a red sector. If a maize line is carries dominant ultila for genes encoding the enzymatic intermediates in the anthocyanin biosynthetic pathway (C2, A1, A2, Bz1 and Bz2) (Roth et al., 1990), but carries a recessive allele at the R locus, transformation of any cell from that line with R will result in red pigment formation. Exemplary lines include Wisconsin 22 which contains the rg-Stadler allele and TR112, a K55 derivative which is r-g, b, P1. Alternatively any genotype of maize can be utilized if the C1 and R alleles are introduced together.

It is further proposed that R gene regulatory regions may be employed in chimeric constructs in order to provide mechanisms for controlling the expression of chimeric genes. More diversity of phenotypic expression is known at the R locus than at any other locus (Coe et al., 1988). It is contemplated that regulatory regions obtained from regions 5′ to the structural R gene would be valuable in directing the expression of genes, e.g., insect resistance, drought resistance, herbicide tolerance or other protein coding regions. For the purposes of the present invention, it is believed that any of the various R gene family members may be successfully employed (e.g., P, S, Lc, etc.). However, the most preferred will generally be Sn (particularly Sn:bol3). Sn is a dominant member of the R gene complex and is functionally similar to the R and B loci in that Sn controls the tissue specific deposition of anthocyanin pigments in certain seedling and plant cells, therefore, its phenotype is similar to R.

A further screenable marker contemplated for use in the present invention is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It is also envisioned that this system may be developed for populational screening for bioluminescence, such as on tissue culture plates, or even for whole plant screening. Where use of a screenable marker gene such as lux or GFP is desired, benefit may be realized by creating a gene fusion between the screenable marker gene and a selectable marker gene, for example, a GFP-NPTII gene fusion. This could allow, for example, selection of transformed cells followed by screening of transgenic plants or seeds.

Other sequences that may be linked to any one of SEQ ID NO.: 1-70 are those that can target to a specific organelle, e.g., to the mitochondria, nucleus, or plastid, within the plant cell. Targeting can be achieved by providing the polypeptide with an appropriate targeting peptide sequence, such as a secretory signal peptide (for secretion or cell wall or membrane targeting, a plastid transit peptide, a chloroplast transit peptide, e.g., the chlorophyll a/b binding protein, a mitochondrial target peptide, a vacuole targeting peptide, or a nuclear targeting peptide, and the like. For example, the small subunit of ribulose bisphosphate carboxylase transit peptide, the EPSPS transit peptide or the dihydrodipicolinic acid synthase transit peptide may be used. For examples of plastid organelle targeting sequences (see WO 00/12732). Plastids are a class of plant organelles derived from proplastids and include chloroplasts, leucoplasts, aravloplasts, and chromoplasts. The plastids are major sites of biosynthesis in plants. In addition to photosynthesis in the chloroplast, plastids are also sites of lipid biosynthesis, nitrate reduction to ammonium, and starch storage. And while plastids contain their own circular genome, most of the proteins localized to the plastids are encoded by the nuclear genome and are imported into the organelle from the cytoplasm.

The choice of the particular DNA segments to be delivered to the recipient cells will often depend on the purpose of the transformation. One of the major purposes of transformation of crop plants is to add some commercially desirable, agronomically important traits to the plant. Such traits include, but are not limited to, herbicide resistance or tolerance; insect resistance or tolerance; disease resistance or tolerance (viral, bacterial, fungal, nematode); stress tolerance and/or resistance, as exemplified by resistance or tolerance to drought, heat, chilling, freezing, excessive moisture, salt stress; oxidative stress; increased yields; food content and makeup; physical appearance; male sterility; drydown; standability; prolificacy; starch properties; oil quantity and quality; and the like. One may desire to incorporate one or more genes conferring any such desirable trait or traits, such as, for example, a gene or genes encoding pathogen resistance.

In certain embodiments, the present invention contemplates the transformation of a recipient cell with more than one advantageous transgene. This includes tranfornation with more than one transcription factor of the present invention or transformation with a combination of one or more of the transcription factors disclosed herein in combination with other genes of interest. Two or more transgenes can be supplied in a single transformation event using either distinct transgene-encoding vectors, or using a single vector incorporating two or more gene coding sequences. Of course, transcription factors of the present invention can be combined with one or more transgenes of any description, such as those conferring herbicide, insect, disease (viral, bacterial, fungal, nematode) or drought resistance, male sterility, drydown, standability, prolificacy, starch properties, oil quantity and quality, or those increasing yield or nutritional quality may be employed as desired.

Improvement of a plant's ability to tolerate various environmental stresses such as, but not limited to, drought, excess moisture, chilling, freezing, high temperature, salt, and oxidative stress, can also be effected through expression of heterologous, or overexpression of homologous genes. Benefits may be realized in terms of increased resistance to freezing temperatures through the introduction of an “antifreeze” protein such as that of the Winter Flounder (Cutler et al., 1989) or synthetic gene derivatives thereof. Improved chilling tolerance may also be conferred through increased expression of glycerol-3-phosphate acetyltransferase in chloroplasts (Murata et al., 1992; Wolter et al., 1992). Resistance to oxidative stress (often exacerbated by conditions such as chilling temperatures in combination with high light intensities) can be conferred by expression of superoxide dismutase (Gupta et al., 1993), and may be improved by glutathione reductase (Bowler et al., 1992). Such strategies may allow for tolerance to freezing in newly emerged fields as well as extending later maturity higher yielding varieties to earlier relative maturity zones.

Plant species may be transformed with the DNA construct of the present invention by the DNA-mediated transformation of plant cell protoplasts and subsequent regeneration of the plant from the transformed protoplasts in accordance with procedures well known in the art.

Any plant tissue capable of subsequent clonal propagation, whether by organogenesis or embryogenesis, may be transformed with a vector of the present invention. The term “organogenesis,” as used herein, means a process by which shoots and roots are developed sequentially from meristematic centers; the term “embryogenesis,” as used herein, means a process by which shoots and roots develop together in a concerted fashion (not sequentially), whether from somatic cells or gametes. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristems, axillary buds, and root meristems), and induced meristem tissue (e.g., cotyledon meristem and ultilane meristem).

Plants of the present invention may take a variety of forms. The plants may be chimeras of transformed cells and non-transformed cells; the plants may be clonal transformants (e.g., all cells transformed to contain the expression cassette); the plants may comprise grafts of transformed and untransformed tissues (e.g., a transformed root stock grafted to an untransformed scion in citrus species). The transformed plants may be propagated by a variety of means, such as by clonal propagation or classical breeding techniques. For example, first generation (or T1) transformed plants may be selfed to give homozygous second generation (or T2) transformed plants, and the T2 plants further propagated through classical breeding techniques. A dominant selectable marker (such as npt II) can be associated with the expression cassette to assist in breeding.

Thus, the present invention provides a transformed (transgenic) plant cell, in planta or ex planta, including a transformed plastid or other organelle, e.g., nucleus, mitochondria or chloroplast. The present invention may be used for transformation of any plant species, including, but not limited to, cells from corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea ultilane), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, duckweed (Lemna), barley, vegetables, ornamentals, and conifers.

Duckweed (Lemna, see WO 00/07210) includes members of the family Lemnaceae. There are known four genera and 34 species of duckweed as follows: genus Lemna (L. aequinoctialis, L. disperma, L. ecuadoriensis, L. gibba, L. japonica, L. minor, L. miniscula, L. obscura, L. perpusilla, L. tenera, L. trisulca, L.turionifera, L. valdiviana); genus Spirodela (S. intermedia, S. polyrrhiza, S. punctata); genus Woffia (Wa. Angusta, Wa. Arrhiza, Wa. Australina, Wa. Borealis, Wa. Brasiliensis, Wa. Columbiana, Wa. Elongata, Wa. Globosa, Wa. Microscopica, Wa. Neglecta) and genus Wofiella (Wl. ultila, Wl. ultilanen, Wl. gladiata, Wl. ultila, Wl. lingulata, Wl. repunda, Wl. rotunda, and Wl. neotropica). Any other genera or species of Lemnaceae, if they exist, are also aspects of the present invention. Lemna gibba, Lemna minor, and Lemna miniscula are preferred, with Lemna minor and Lemna miniscula being most preferred. Lemna species can be classified using the taxonomic scheme described by Landolt, Biosystematic Investigation on the Family of Duckweeds: The family of Lemnaceae—A Monograph Study. Geobatanischen Institut ETH, Stiftung Rubel, Zurich (1986)).

Vegetables within the scope of the invention include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata), Douglas-fir (Pseudotsuga menziesii); Western hemlock (Tsuga ultilane); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Leguminous plants include beans and peas. Beans include guar, locust bean, fenugreek, soybean, garden beans, cowpea, mungbean, lima bean, fava bean, lentils, chickpea, etc. Legumes include, but are not limited to, Arachis, e.g., peanuts, Vicia, e.g., crown vetch, hairy vetch, adzuki bean, mung bean, and chickpea, Lupinus, e.g., lupine, trifolium, Phaseolus, e.g., common bean and lima bean, Pisum, e.g., field bean, Melilotus, e.g., clover, Medicago, e.g., alfalfa, Lotus, e.g., trefoil, lens, e.g., lentil, and false indigo. Preferred forage and turf grass for use in the methods of the invention include alfalfa, orchard grass, tall fescue, perennial ryegrass, creeping bent grass, and redtop.

Papaya, garlic, pea, peach, pepper, petunia, strawberry, sorghum, sweet potato, turnip, safflower, corn, pea, endive, gourd, grape, snap bean, chicory, cotton, tobacco, aubergine, beet, buckwheat, broad bean, nectarine, avocado, mango, banana, groundnut, potato, peanut, lettuce, pineapple, spinach, squash, sugarbeet, sugarcane, sweet corn, chrysanthemum.

Other plants within the scope of the invention include Acacia, aneth, artichoke, arugula, blackberry, canola, cilantro, clementines, escarole, eucalyptus, fennel, grapefruit, honey dew, jicama, kiwifruit, lemon, lime, mushroom, nut, okra, orange, parsley, persimmon, plantain, pomegranate, poplar, radiata pine, radicchio, Southern pine, sweetgum, tangerine, triticale, vine, yams, apple, pear, quince, cherry, apricot, melon, hemp, buckwheat, grape, raspberry, chenopodium, blueberry, nectarine, peach, plum, strawberry, watermelon, eggplant, pepper, cauliflower, Brassica, e.g., broccoli, cabbage, ultilan sprouts, onion, carrot, leek, beet, broad bean, celery, radish, pumpkin, endive, gourd, garlic, snapbean, spinach, squash, turnip, ultilane, and zucchini.

Ornamental plants within the scope of the invention include impatiens, Begonia, Pelargonium, Viola, Cyclamen, Verbena, Vinca, Tagetes, Primula, Saint Paulia, Agertum, Amaranthus, Antihirrhinum, Aquilegia, Cineraria, Clover, Cosmo, Cowpea, Dahlia, Datura, Delphinium, Gerbera, Gladiolus, Gloxinia, Hippeastrum, Mesembryanthemum, Salpiglossos, and Zinnia. Other plants within the scope of the invention are shown in Table 1 (above).

Typically, transgenic plants of the present invention are crop plants and in particular cereals (for example, corn, alfalfa, sunflower, rice, Brassica, canola, soybean, barley, soybean, sugarbeet, cotton, safflower, peanut, sorghum, wheat, millet, tobacco, etc.), and even more typicaly corn, rice and soybean.

Transformation of plants can be undertaken with a single DNA molecule or multiple DNA molecules (i.e., co-transformation), and both these techniques are suitable for use with the expression cassettes of the present invention. Numerous transformation vectors are available for plant transformation, and the expression cassettes of this invention can be used in conjunction with any such vectors. The selection of vector will depend upon the preferred transformation technique and the target species for transformation.

A variety of techniques are available and known to those skilled in the art for introduction of constructs into a plant cell host. These techniques generally include transformation with DNA employing A. tumefaciens or A. rhizogenes as the transforming agent, liposomes, PEG precipitation, electroporation, DNA injection, direct DNA uptake, microprojectile bombardment, particle acceleration, and the like (See, for example, EP 295959 and EP 138341) (see below). However, cells other than plant cells may be transformed with the expression cassettes of the invention. The general descriptions of plant expression vectors and reporter genes, and Agrobacterium and Agrobacterium-mediated gene transfer, can be found in Gruber et al. (1993).

Expression vectors containing genomic or synthetic fragments can be introduced into protoplasts or into intact tissues or isolated cells. Typically expression vectors are introduced into intact tissue. General methods of culturing plant tissues are provided for example by Maki et al., (1993); and by Phillips et al. (1988). Typically, expression vectors are introduced into maize or other plant tissues using a direct gene transfer method such as microprojectile-mediated delivery, DNA injection, electroporation and the like. Further, expression vectors are introduced into plant tissues using the microprojectile media delivery with the biolistic device. See, for example, Tomes et al. (1995). The vectors of the invention can not only be used for expression of structural genes but may also be used in exon-trap cloning, or promoter trap procedures to detect differential gene expression in varieties of tissues, (Lindsey et al., 1993; Auch & Reth et al.).

In one embodiment, binary type vectors of Ti and Ri plasmids of Agrobacterium spp. Ti-derived vectors transform a wide variety of higher plants, including monocotyledonous and dicotyledonous plants, such as soybean, cotton, rape, tobacco, and rice (Pacciotti et al., 1985: Byrne et al., 1987; Sukhapinda et al., 1987; Park et al., 1985: Hiei et al., 1994). The use of T-DNA to transform plant cells has received extensive study and is amply described (EP 120516; Hoekema, 1985; Knauf, et al., 1983; and An et al., 1985). For introduction into plants, the chimeric genes of the invention can be inserted into binary vectors as described in the examples.

Other transformation methods are available to those skilled in the art, such as direct uptake of foreign DNA constructs (see EP 295959), techniques of electroporation (Fromm et al., 1986) or high velocity ballistic bombardment with metal particles coated with the nucleic acid constructs (Kline et al., 1987, and U.S. Pat. No. 4,945,050). Once transformed, the cells can be regenerated by those skilled in the art. Of particular relevance are the recently described methods to transform foreign genes into commercially important crops, such as rapeseed (De Block et al., 1989), sunflower (Everett et al., 1987), soybean (McCabe et al., 1988; Hinchee et al., 1988; Chee et al., 1986; Christou et al., 1988; EP 301749), rice (Hiei et al., 1994), and corn (Gordon Kamm et al., 1990; Fromm et al., 1990).

Those skilled in the art will appreciate that the choice of method might depend on the type of plant, i.e., monocotyledonous or dicotyledonous, targeted for transformation. Suitable methods of transforming plant cells include, but are not limited to, microinjection (Crossway et al., 1986), electroporation (Riggs et al., 1986), Agrobacterium-mediated transformation (Hinchee et al., 1988), direct gene transfer (Paszkowski et al., 1984), and ballistic particle acceleration using devices available from Agracetus, Inc., Madison, Wis. And BioRad, Hercules, Calif. (see, for example, Sanford et al., 1987, U.S. Pat. No. 4,945,050; and McCabe et al., 1988). Also see, Weissinger et al., 1988; Sanford et al., 1987 (onion); Christou et al., 1988 (soybean); McCabe et al., 1988 (soybean); Datta et al., 1990 (rice); Klein et al., 1988 (maize); Klein et al., 1989 (maize); Fromm et al., 1990 (maize); and Gordon-Kamm et al., 1990 (maize); Svab et al., 1990 (tobacco chloroplast); Koziel et al., 1993 (maize); Shimamoto et al., 1989 (rice); Christou et al., 1991 (rice); European Patent Application EP 0 332 581 (orchardgrass and other Pooideae); Vasil et al., 1993 (wheat); Weeks et al., 1993 (wheat). In one embodiment, the protoplast transformation method for maize is employed (European Patent Application EP 0 292 435, U.S. Pat. No. 5,350,689).

In another embodiment, a nucleotide sequence of the present invention is directly transformed into the plastid genome. Plastid transformation technology is extensively described in U.S. Pat. Nos. 5,451,513, 5,545,817, and 5,545,818, in PCT application No. WO 95/16783, and in McBride et al., 1994. The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate orthologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab et al., 1990; Staub et al., 1992). This resulted in stable homoplasmic transformants at a frequency of approximately one per 100 bombardments of target leaves. The presence of cloning sites between these markers allowed creation of a plastid targeting vector for introduction of foreign genes (Staub et al., 1993). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3N-adenyltransferase (Svab et al., 1993). Other selectable markers useful for plastid transformation are known in the art and encompassed within the scope of the invention.

Typically, approximately 15-20 cell division cycles following transformation are required to reach a homoplastidic state. Plastid expression, in which genes are inserted by orthologous recombination into all of the several thousand copies of the circular plastid genome present in each plant cell, takes advantage of the enormous copy number advantage over nuclear-expressed genes to permit expression levels that can readily exceed 10% of the total soluble plant protein. In a preferred embodiment, a nucleotide sequence of the present invention is inserted into a plastid targeting vector and transformed into the plastid genome of a desired plant host. Plants homoplastic for plastid genomes containing a nucleotide sequence of the present invention are obtained, and are preferentially capable of high expression of the nucleotide sequence.

Agrobacterium tumefaciens cells containing a vector comprising an expression cassette of the present invention, wherein the vector comprises a Ti plasmid, are useful in methods of making transformed plants. Plant cells are infected with an Agrobacterium tumefaciens as described above to produce a transformed plant cell, and then a plant is regenerated from the transformed plant cell. Numerous Agrobacterium vector systems useful in carrying out the present invention are known.

Methods using either a form of direct gene transfer or Agrobacterium-mediated transfer usually, but not necessarily, are undertaken with a selectable marker which may provide resistance to an antibiotic (e.g., kanamycin, hygromycin or methotrexate) or a herbicide (e.g., phosphinothricin). The choice of selectable marker for plant transformation is not, however, critical to the invention.

For certain plant species, different antibiotic or herbicide selection markers may be preferred. Selection markers used routinely in transformation include the nptII gene which confers resistance to kanamycin and related antibiotics (Messing & Vierra, 1982; Bevan et al., 1983), the bar gene which confers resistance to the herbicide phosphinothricin (White et al., 1990, Spencer et al., 1990), the hph gene which confers resistance to the antibiotic hygromycin (Blochinger & Diggelmann), and the dhfr gene, which confers resistance to methotrexate (Bourouis et al., 1983).

One such vector useful for direct gene transfer techniques in combination with selection by the herbicide Basta (or phosphinothricin) is pCIB3064. This vector is based on the plasmid pCIB246, which comprises the CaMV 35S promoter in operational fusion to the E. coli GUS gene and the CaMV 35S transcriptional terminator and is described in the PCT published application WO 93/07278, herein incorporated by reference. One gene useful for conferring resistance to phosphinothricin is the bar gene from Streptomyces viridochromogenes (Thompson et al., 1987). This vector is suitable for the cloning of plant expression cassettes containing their own regulatory signals.

An additional transformation vector is pSOG35 which utilizes the E. coli gene dihydrofolate reductase (DHFR) as a selectable marker conferring resistance to methotrexate. PCR was used to amplify the 35S promoter (about 800 bp), intron 6 from the maize Adh1 gene (about 550 bp) and 18 bp of the GUS untranslated leader sequence from pSOG10. A 250 bp fragment encoding the E. coli dihydrofolate reductase type II gene was also amplified by PCR and these two PCR fragments were assembled with a SacI-PstI fragment from pBI221 (Clontech) which comprised the pUC19 vector backbone and the nopaline synthase terminator. Assembly of these fragments generated pSOG19 which contains the 35S promoter in fusion with the intron 6 sequence, the GUS leader, the DHFR gene and the nopaline synthase terminator. Replacement of the GUS leader in pSOG19 with the leader sequence from Maize Chlorotic Mottle Virus check (MCMV) generated the vector pSOG35. pSOG19 and pSOG35 carry the pUC-derived gene for ampicillin resistance and have HindIII, SphI, PstI and EcoRI sites available for the cloning of foreign sequences.

Transgenic plant cells are then placed in an appropriate selective medium for selection of transgenic cells which are then grown to callus. Shoots are grown from callus and plantlets generated from the shoot by growing in rooting medium. The various constructs normally will be joined to a marker for selection in plant cells. Conveniently, the marker may be resistance to a biocide (particularly an antibiotic, such as kanamycin, G418, bleomycin, hygromycin, chloramphenicol, herbicide, or the like). The particular marker used will allow for selection of transformed cells as compared to cells lacking the DNA which has been introduced. Components of DNA constructs including transcription cassettes of this invention may be prepared from sequences which are native (endogenous) or foreign (exogenous) to the host. By “foreign” it is meant that the sequence is not found in the wild-type host into which the construct is introduced. Heterologous constructs will contain at least one region which is not native to the gene from which the transcription-initiation-region is derived.

To confirm the presence of the transgenes in transgenic cells and plants, a variety of assays may be performed. Such assays include, for example, “molecular biological” assays well known to those of skill in the art, such as Southern and Northern blotting, in situ hybridization and nucleic acid-based amplification methods such as PCR or RT-PCR; “biochemical” assays, such as detecting the presence of a protein product, e.g., by immunological means (ELISAs and Western blots) or by enzymatic function; plant part assays, such as leaf or root assays; and also, by analyzing the phenotype of the whole regenerated plant, e.g., for disease or pest resistance.

DNA may be isolated from cell lines or any plant parts to determine the presence of the preselected nucleic acid segment through the use of techniques well known to those skilled in the art. Note that intact sequences will not always be present, presumably due to rearrangement or deletion of sequences in the cell. The presence of nucleic acid elements introduced through the methods of this invention may be determined by polymerase chain reaction (PCR). Using this technique discreet fragments of nucleic acid are amplified and detected by gel electrophoresis. This type of analysis permits one to determine whether a preselected nucleic acid segment is present in a stable transformant, but does not prove integration of the introduced preselected nucleic acid segment into the host cell genome. In addition, it is not possible using PCR techniques to determine whether transformants have exogenous genes introduced into different sites in the genome, i.e., whether transformants are of independent origin. It is contemplated that using PCR techniques it would be possible to clone fragments of the host genomic DNA adjacent to an introduced preselected DNA segment.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition it is possible through Southern hybridization to demonstrate the presence of introduced preselected DNA segments in high molecular weight DNA, i.e., confirm that the introduced preselected DNA segment has been integrated into the host cell genome. The technique of Southern hybridization provides information that is obtained using PCR, e.g., the presence of a preselected DNA segment, but also demonstrates integration into the genome and characterizes each individual transformant.

It is contemplated that using the techniques of dot or slot blot hybridization which are modifications of Southern hybridization techniques one could obtain the same information that is derived from PCR, e.g., the presence of a preselected DNA segment.

Both PCR and Southern hybridization techniques can be used to demonstrate transmission of a preselected DNA segment to progeny. In most instances the characteristic Southern hybridization pattern for a given transformant will segregate in progeny as one or more Mendelian genes (Spencer et al., 1990; Laursen et al., 1994) indicating stable inheritance of the gene.

Whereas DNA analysis techniques may be conducted using DNA isolated from any part of a plant, RNA may only be expressed in particular cells or tissue types and hence it will be necessary to prepare RNA for analysis from these tissues. PCR techniques may also be used for detection and quantitation of RNA produced from introduced preselected DNA segments. In this application of PCR it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species can also be determined using dot or slot blot Northern hybridizations. These techniques are modifications of Northern blotting and will only demonstrate the presence or absence of an RNA species.

While Southern blotting and PCR may be used to detect the preselected DNA segment in question, they do not provide information as to whether the preselected DNA segment is being expressed. Expression may be evaluated by specifically identifying the protein products of the introduced preselected DNA segments or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures may also be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed.

Very frequently the expression of a gene product is determined by evaluating the phenotypic results of its expression. These assays also may take many forms including but not limited to analyzing changes in the chemical composition, morphology, or physiological properties of the plant. Morphological changes may include greater stature or thicker stalks. Most often changes in response of plants or plant parts to imposed treatments are evaluated under carefully controlled conditions termed bioassays.

Once an expression cassette of the invention has been transformed into a particular plant species, it may be propagated in that species or moved into other varieties of the same species, particularly including commercial varieties, using traditional breeding techniques. Particularly preferred plants of the invention include the agronomically important crops listed above. The genetic properties engineered into the transgenic seeds and plants described above are passed on by sexual reproduction and can thus be maintained and propagated in progeny plants. The present invention also relates to a transgenic plant cell, tissue, organ, seed or plant part obtained from the transgenic plant. Also included within the invention are transgenic descendants of the plant as well as transgenic plant cells, tissues, organs, seeds and plant parts obtained from the descendants.

Preferably, the expression cassette containing any of the transcription factors of the present invention in the transgenic plant is sexually transmitted. In one preferred embodiment, the coding sequence is sexually transmitted through a complete normal sexual cycle of the R0 plant to the R1 generation. Additionally preferred, the expression cassette is expressed in the cells, tissues, seeds or plant of a transgenic plant in an amount that is different than the amount in the cells, tissues, seeds or plant of a plant which only differs in that the expression cassette is absent.

The transgenic plants produced herein are thus expected to be useful for a variety of commercial and research purposes. Transgenic plants can be created for use in traditional agriculture to possess traits beneficial to the grower (e.g., agronomic traits such as resistance to water deficit, pest resistance, herbicide resistance or increased yield), beneficial to the consumer of the grain harvested from the plant (e.g., improved nutritive content in human food or animal feed; increased vitamin, amino acid, and antioxidant content; the production of antibodies (passive immunization) and nutriceuticals), or beneficial to the food processor (e.g., improved processing traits). In such uses, the plants are generally grown for the use of their grain in human or animal foods.

Transgenic plants may also find use in the commercial manufacture of proteins or other molecules, where the molecule of interest is extracted or purified from plant parts, seeds, and the like. Cells or tissue from the plants may also be cultured, grown in vitro, or fermented to manufacture such molecules.

The transgenic plants may also be used in commercial breeding programs, or may be crossed or bred to plants of related crop species. Improvements encoded by the expression cassette may be transferred, e.g., from maize cells to cells of other species, e.g., by protoplast fusion.

The transgenic plants may have many uses in research or breeding, including creation of new mutant plants through insertional mutagenesis, in order to identify beneficial mutants that might later be created by traditional mutation and selection. An example would be the introduction of a recombinant DNA sequence encoding a transposable element that may be used for generating genetic variation. The methods of the invention may also be used to create plants having unique “signature sequences” or other marker sequences which can be used to identify proprietary lines or varieties.

Thus, the transgenic plants and seeds according to the invention can be used in plant breeding which aims at the development of plants with improved properties conferred by the expression cassette, such as tolerance of drought, disease, or other stresses. The various breeding steps are characterized by well-defined human intervention such as selecting the lines to be crossed, directing pollination of the parental lines, or selecting appropriate descendant plants. Depending on the desired properties different breeding measures are taken. The relevant techniques are well known in the art and include but are not limited to hybridization, inbreeding, backcross breeding, ultilane breeding, variety blend, interspecific hybridization, aneuploid techniques, etc. Hybridization techniques also include the sterilization of plants to yield male or female sterile plants by mechanical, chemical or biochemical means. Cross pollination of a male sterile plant with pollen of a different line assures that the genome of the male sterile but female fertile plant will uniformly obtain properties of both parental lines. Thus, the transgenic seeds and plants according to the invention can be used for the breeding of improved plant lines that for example increase the effectiveness of conventional methods such as herbicide or pesticide treatment or allow to dispense with said methods due to their modified genetic properties.

Polynucleotides derived from the nucleic acid molecules of the present invention having any of the nucleotide sequences of SEQ ID NO: 1 to SEQ ID NO: 70 are useful to detect the presence in a test sample of at least one copy of a nucleotide sequence containing the same or substantially the same sequence, or a fragment, complement, or variant thereof. The sequence of the probes and/or primers of the instant invention need not be identical to those provided in the Sequence Listing or the complements thereof. Some variation in probe or primer sequence and/or length can allow additional family members to be detected, as well as orthologous genes and more taxonomically distant related sequences. Similarly probes and/or primers of the invention can include additional nucleotides that serve as a label for detecting duplexes, for isolation of duplexed polynucleotides, or for cloning purposes.

Preferred probes and primers of the invention include isolated, purified, or recombinant polynucleotides containing a contiguous span of between at least 12 to at least 1000 nucleotides of any of SEQ ID NO: 1 to SEQ ID NO: 70 or the complements thereof, with each individual number of nucleotides within this range also being part of the invention. Preferred are isolated, purified, or recombinant polynucleotides containing a contiguous span of at least 12, 15, 18, 20, 25, 30, 35, 40, 50, 60, 70, 80, 90, 100, 150, 200, 300, 400, 500, 750, or 1000 nucleotides of any of SEQ ID NO: 1 to SEQ ID NO: 70 or the complements thereof. The appropriate length for primers and probes will vary depending on the application. For use as PCR primers, probes are 12-40 nucleotides, preferably 18-30 nucleotides long. For use in mapping, probes are 50 to 500 nucleotides, preferably 100-250 nucleotides long. For use in Southern hybridizations, probes as long as several kilobases can be used. The appropriate length for primers and probes under a particular set of assay conditions may be empirically determined by one of skill in the art.

The primers and probes can be prepared by any suitable method, including, for example, cloning and restriction of appropriate sequences and direct chemical synthesis by a method such as the phosphodiester method of Narang et al. Meth Enzymol 68: 90 (1979), the diethylphosphoramidite method, the triester method of Matteucci et al. J Am Chem Soc 103: 3185 (1981), or according to Urdea et al. Proc Natl Acad 80: 7461 (1981), the solid support method described in EP 0 707 592, or using commercially available automated oligonucleotide synthesizers.

Detection probes are generally nucleotide sequences or uncharged nucleotide analogs such as, for example peptide nucleotides which are disclosed in International Patent Application WO 92/20702, morpholino analogs which are described in U.S. Pat. Nos. 5,185,444, 5,034,506 and 5,142,047. The probe may have to be rendered “non-extendable” such that additional dNTPs cannot be added to the probe. Analogs are usually non-extendable, and nucleotide probes can be rendered non-extendable by modifying the 3′ end of the probe such that the hydroxyl group is no longer capable of participating in elongation. For example, the 3′ end of the probe can be functionalized with the capture or detection label to thereby consume or otherwise block the hydroxyl group. Alternatively, the 3′ hydroxyl group simply can be cleaved, replaced or modified so as to render the probe non-extendable.

Any of the polynucleotides of the present invention can be labeled, if desired, by incorporating a label detectable by spectroscopic, photochemical, biochemical, immunochemical, or chemical means. For example, useful labels include radioactive substances (³²P, ³⁵S, ³H, ¹²⁵I), fluorescent dyes (5-bromodesoxyuridine, fluorescein, acetylaminofluorene, digoxigenin) or biotin. Preferably, polynucleotides are labeled at their 3′ and 5′ ends. Examples of non-radioactive labeling of nucleotide fragments are described in the French patent No. FR-7810975 and by Urdea et al. Nuc Acids Res 16:4937 (1988). In addition, the probes according to the present invention may have structural characteristics such that they allow the signal amplification, such structural characteristics being, for example, branched DNA probes as described in EP 0 225 807.

A label can also be used to capture the primer so as to facilitate the immobilization of either the primer or a primer extension product, such as amplified DNA, on a solid support. A capture label is attached to the primers or probes and can be a specific binding member that forms a binding pair with the solid's phase reagent's specific binding member, for example biotin and streptavidin. Therefore depending upon the type of label carried by a polynucleotide or a probe, it may be employed to capture or to detect the target DNA. Further, it will be understood that the polynucleotides, primers or probes provided herein, may, themselves, serve as the capture label. For example, in the case where a solid phase reagent's binding member is a nucleotide sequence, it may be selected such that it binds a complementary portion of a primer or probe to thereby immobilize the primer or probe to the solid phase. In cases where a polynucleotide probe itself serves as the binding member, those skilled in the art will recognize that the probe will contain a sequence or “tail” that is not complementary to the target. In the case where a polynucleotide primer itself serves as the capture label, at least a portion of the primer will be free to hybridize with a nucleotide on a solid phase. DNA labeling techniques are well known in the art.

Any of the polynucleotides, primers and probes of the present invention can be conveniently immobilized on a solid support. Solid supports are known to those skilled in the art and include the walls of wells of a reaction tray, test tubes, polystyrene beads, magnetic beads, nitrocellulose strips, membranes, microparticles such as latex particles, sheep (or other animal) red blood cells, duracytes and others. The solid support is not critical and can be selected by one skilled in the art. Thus, latex particles, microparticles, magnetic or non-magnetic beads, membranes, plastic tubes, walls of microtiter wells, glass or silicon chips, sheep (or other suitable animal's) red blood cells and duracytes are all suitable examples. Suitable methods for immobilizing nucleotides on solid phases include ionic, hydrophobic, covalent interactions and the like. The polynucleotides of the invention can be attached to or immobilized on a solid support individually or in groups of at least 2, 5, 8, 10, 12, 15, 20, or 25 distinct polynucleotides of the invention to a single solid support. In addition, polynucleotides other than those of the invention may be attached to the same solid support as one or more polynucleotides of the invention.

A solid support, as used herein, refers to any material that is insoluble, or can be made insoluble by a subsequent reaction. The solid support can be chosen for its intrinsic ability to attract and immobilize the capture reagent. Alternatively, the solid phase can retain an additional receptor that has the ability to attract and immobilize the capture reagent. The additional receptor can include a charged substance that is oppositely charged with respect to the capture reagent itself or to a charged substance conjugated to the capture reagent. As yet another alternative, the receptor molecule can be any specific binding member which is immobilized upon (attached to) the solid support and which has the ability to immobilize the capture reagent through a specific binding reaction. The receptor molecule enables the indirect binding of the capture reagent to a solid support material before the performance of the assay or during the performance of the assay.

The polynucleotides of the invention that are expressed or repressed in response to environmental stimuli such as, for example, biotic or abiotic stress or treatment with chemicals or pathogens or at different developmental stages can be identified by employing an array of nucleic acid samples, e.g., each sample having a plurality of oligonucleotides, and each plurality corresponding to a different plant gene, on a solid substrate, e.g., a DNA chip, and probes corresponding to nucleic acid expressed in, for example, one or more plant tissues and/or at one or more developmental stages, e.g., probes corresponding to nucleic acid expressed in seed of a plant relative to control nucleic acid from sources other than seed. Thus, genes that are upregulated or downregulated in the majority of tissues at a majority of developmental stages, or upregulated or downregulated in one tissue such as in seed, can be systematically identified. The probes may also correspond to nucleic acid expressed in respone to a defined treatment such as, for example, a treatment with a variety of plant hormones or the exposure to specific environmental conditions involving, for example, an abiotic stress or exposure to light.

Specifically, labeled rice cRNA probes were hybridized to the rice DNA array, expression levels were determined by laser scanning and then rice genes were identified that had a particular expression pattern. The rice oligonucleotide probe array consists of probes from over 18,000 unique rice genes, which covers approximately 40-50% of the genome. This genome array permits a broader, more complete and less biased analysis of gene expression.

As described herein, GeneChip® technology was utilized to discover rice transcription factor genes that are preferentially (or exclusively) expressed in respone to a defined exposure to specific abiotic stresses including, for example, drought, cold, salt and osmotic stresses, such as set forth in Table 1, below.

Using this approach, 70 genes were identified, the expression of which was altered, e.g., specifically elevated or repressed, in in response to drought, cold, salt and osmotic stresses or various combinations thereof.

Consequently, the invention also deals with a method for detecting the presence of a polynucleotide including a nucleotide sequence which is substantially similar, and preferably has at least between 70% and 99%, sequence identity to any one of SEQ ID NO.: 1-70 or a fragment or a variant thereof, or a complementary sequence thereto in a sample, the method including the following steps of:
(a) bringing into contact a nucleotide probe or a plurality of nucleotide probes which can hybridize with polynucleotide having a nucleotide sequence which is substantially similar, and preferably has at least between 70% and 99%, sequence identity to any one of SEQ ID NO.: 1-70 or a fragment or a variant thereof, or a complementary sequence thereto and the sample to be assayed.
(b) detecting the hybrid complex formed between the probe and a nucleotide in the sample.

The invention further concerns a kit for detecting the presence of a polynucleotide including a nucleotide sequence which is substantially similar, and preferably has at least between 70% and 99%, sequence identity to any one of SEQ ID NO.: 1-70 or a fragment or a variant thereof, or a complementary sequence thereto in a sample, the kit including a nucleotide probe or a plurality of nucleotide probes which can hybridize with a nucleotide sequence included in a polynucleotide including a nucleotide sequence which is substantially similar, and preferably has at least between 70% and 99%, sequence identity to any one of SEQ ID NO.: 1-70 or a fragment or a variant thereof, or a complementary sequence thereto and, optionally, the reagents necessary for performing the hybridization reaction.

In a first preferred embodiment of this detection method and kit, the nucleotide probe or the plurality of nucleotide probes are labeled with a detectable molecule. In a second preferred embodiment of the method and kit, the nucleotide probe or the plurality of nucleotide probes has been immuobilized on a substrate.

The invention also provides a computer readable medium having stored thereon a data structure containing nucleic acid sequences having at least 70% sequence identity to a nucleic acid sequence selected from those listed in SEQ ID Nos: 1-70, as well as complementary, ortholog, and variant sequences thereof. Storage and use of nucleic acid sequences on a computer readable medium is well known in the art. (See for example U.S. Pat. Nos. 6,023,659; 5,867,402; 5,795,716) Examples of such medium include, but are not limited to, magnetic tape, optical disk, CD-ROM, random access memory, volatile memory, non-volatile memory and bubble memory. Accordingly, the nucleic acid sequences contained on the computer readable medium may be compared through use of a module that receives the sequence information and compares it to other sequence information. Examples of other sequences to which the nucleic acid sequences of the invention may be compared include those maintained by the National Center for Biotechnology Information (NCBI)(http://www.ncbi.nlm.nih.gov/) and the Swiss Protein Data Bank. A computer is an example of such a module that can read and compare nucleic acid sequence information. Accordingly, the invention also provides the method of comparing a nucleic acid sequence of the invention to another sequence. For example, a sequence of the invention may be submitted to the NCBI for a Blast search as described herein where the sequence is compared to sequence information contained within the NCBI database and a comparison is returned. The invention also provides nucleic acid sequence information in a computer readable medium that allows the encoded polypeptide to be optimized for a desired property. Examples of such properties include, but are not limited to, increased or decreased: thermal stability, chemical stability, hydrophylicity, hydrophobicity, and the like. Methods for the use of computers to model polypeptides and polynucleotides having altered activities are well known in the art and have been reviewed. (Lesyng et al., 1993; Surles et al., 1994; Koehl et al., 1996; Rossi et al., 2001).

The following examples are intended to provide illustrations of the application of the present invention. The following examples are not intended to completely define or otherwise limit the scope of the invention. Furthermore, we claim the embodiments and sequences disclosed and/or claimed in the aforementioned priority applications.

### EXAMPLES

### Example 1

### Identification of 402 Known and Putative Transcription Factors

Potential stress-related genes which encode known or putative transcription factors on the Arabidopsis GeneChip (Affymetrix, Santa Clara, Calif.) were identified based on the annotation associated with probesets on the chip. Additional genes were identified by searching for conserved domains. The nucleotide and amino acid sequences from conserved domains for AP2/EREBPs, Myb proteins, bZIPs and WRKY zinc finger proteins was used to blast against the TIGR Arabidopsis database (ftp://ftp.tigr.org/pub/data/a_thaliana/ath1/PSEUDOMOLECULES/), using blastn, blastx and blastp programs (Altschul et al., 1997), to generate the entire list of the known or putative transcription factor genes of these families. Homologues (E-value less than 1E-20) of the list members represented on the GeneChip were included in the analysis.

For Arabidopsis GeneChip experiments, RNA samples were extracted, and subsequent cDNA synthesis, array hybridization and overall intensity normalization for all the arrays for the entire probe sets were performed as described by Zhu et al. (2001a). The average difference (expression level) for the selected 402 genes was then extracted from the data. Any average difference that was less than 5 was floored to 5. Then, the fold change was calculated for each gene by dividing the average difference from various stress treated samples by that from the corresponding mock-control samples. Genes with average differences equal to 5 and which were called “Absent” across all the experiments were eliminated from further analysis. In these stress response experiments, the logarithms (base 2) of the fold change values for each gene were subjected to normalization across all the samples. In the case of studying developmental control and organ specific gene expression, the floored average difference, rather than fold change, was directly subjected to log₂ transformation, followed by mean centering across each gene. All the processed data were then subjected to the self-organizing map algorithm, followed by complete linkage hierarchical clustering of both genes and experiments, using Cluster/Treeview (Eisen et al., 1998), or to the self-organizing maps algorithm for genes using GeneCluster 1.0 (Tamoyo et al. 1999).

Four week old Columbia wild type plants and various mutant and transgenic plants were infected with Psm ES4326 (10⁶ cfu) for 30 hours. The infected leaf samples were then collected, and subjected to GeneChip analysis. Transcription factor genes that are induced by at least 2-fold in wild type plants following Psm ES4326 infection (with present call after infection) were identified. The fold change was calculated by dividing the average difference from mutant or transgenic plant samples by that from the wild-type plant sample. Then the logarithms (base 2) of the fold change values were subjected to cluster analysis as described above.

The cluster of cold inducible genes was selected based on only one time point, either early or late cold treatment. Three-week old Columbia wild type plants grown on sterilized MS agar medium at 22° C. under 12 hr/12 hr light/dark cycle were transferred to fresh MS liquid medium for several days of equilibration before treatment. Salt, osmotic and cold stresses were then applied by replacing the medium with new MS medium containing 100 mM NaCl or 200 mM mannitol or incubating at 4° C. Tissues from control and treated aerial or root portions of the plants were then collected, RNA purified and then subjected to GeneChip analysis. Genes from the Arabidopsis GeneChip that were induced by at least 2-fold following any stress treatment described above (with present call after treatment) were selected. Then the average differences of these genes were log₂-transformed, mean-centered and subjected to Eisen's Cluster/Treeview program as described above. Clusters of genes whose expression was preferentially induced by 3-hour (the early transcription factor gene cluster) and 27-hour cold treatments (late response gene cluster), were identified.

Genes within the pathogen inducible cluster were identified as follows. First, genes from the Arabidopsis GeneChip that were induced by at least 2-fold following any pathogen infection, and at any time point (with present call after treatment) were selected. Then, the fold change was calculated for each gene by dividing the average difference from various pathogen treated samples by that from the corresponding mock-treated samples, followed by log₂-transformation. The processed data were directly subjected to Eisen's Cluster/Treeview program as described above. Clusters of genes whose expression was induced by all pathogens at all time points were identified.

A total of 402 potential stress-related genes that encode known or putative transcription factors were selected for this study from ˜8,300 genes (corresponding to approximately one third of the genome) covered by the Arabidopsis GeneChip. These genes included 63 AP2/EREBP genes, 121 AtMyb genes, 34 bZIP genes, 152 members of the diverse zinc finger gene classes, 12 AtHD-ZIP genes, and 21 IAA/AXR genes. The complex zinc finger gene classes could be further divided into distinct zinc finger gene families based on their structural features. These families included plant-specific WRKY (Eulgem et al., 2000) and Dof proteins (Yanagisawa and Schmidt, 1999); GATA type zinc finger proteins; and ring zinc finger proteins (Jensen et al., 1998). Although some zinc finger proteins, for example the RING zinc finger proteins, might be involved in protein-protein interactions rather than direct DNA binding, they were included in this study because some studies have shown that they too can be involved in transcriptional regulation of gene expression (Borden, 2000; Capili et al., 2001).

Expression levels of these 402 transcription factor genes in various organs, at different developmental stages, and under various biotic and abiotic stresses were monitored. A 2-dimensional transcription matrix (genes vs. treatments or developmental stages/tissues), describing the changes in the mRNA level of the 402 transcription factor genes, was constructed for these experiments. The data represent 19 independent experiments, with samples derived from different organs such as roots, leaves, inflorescence stems, flowers, siliques and at different developmental stages (Zhu et al, 2001a), and more than 80 experiments representing 57 independent treatments with cold, salt, osmoticum, wounding, jasmonic acid (JA) and different types of pathogens, at different time points. To make results comparable across all the experiments in the study of stress response, transcript levels of the stress-treated samples were compared to those of the corresponding mock-treated samples, and fold-change values were used for further clustering analysis. Further information regarding Arabidopsis transcription factors can be found in Chen et al., Plant Cell 14:559-574,2002.

### Example 2

### Identification of Rice Transcription Factors

To obtain rice orthologues of the Arabidopsis transcription factor genes, the full-length cDNA sequences of the 402 Arabidopsis transcription factor genes were used to blast against the internal rice database composed of about 45,000 predicated full-length rice genes, using blastn, with the NCBI default settings (Expectation: 10, Alignment: 20). The blast results were filtered by a custom Perl script, and only the best hits for each entry were retained. Then the full-length sequences of the top hits were retrieved from rice database and used to blast back against all the 402 Arabidopsis transcription factor gene sequences. The top blast hits were obtained by the same method as described above. The two reciprocal blast results were compared, and only those rice genes which matched the same Arabidopsis TF gene in the reciprocal blast search were retained and considered to be the true rice orthologues of the corresponding Arabidopsis transcription factor genes.

A total of 70 rice transcription factor orthologues were identified. Of the rice transcription factors identified, 7 are AP2/EREBP transcription factors (SEQ ID NO.: 20, 23, 33, 36, 40, 44, and 47), 4 are bZIP transcription factors SEQ ID NO.: 6, 27, 45, and 48), 2 are Dof-zinc finger proteins (SEQ ID NO.: 1 and 17), 5 are homeodomain-leucine zippers (SEQ ID NO.: 18, 30, 32, 52, and 53) 2 are IAA type transcription factors (SEQ ID NO.: 31 and 42), 14 are Myb transcription factors (SEQ [D NO.: 5, 7, 10, 14, 15, 16, 21, 25, 26, 34, 35, 37, 50, and 51) 2 are WRKY transcription factors (SEQ ID NO.: 2 and 43) and 4 are zinc finger proteins (8, 11, 13, and 22).

### Example 3

### Expression Profiling of Rice Transcription Factors

In addition, expression of selected rice transcription factor genes was examined using a proprietary rice GeneChip® Rice Genome Array (Affymetrix, Santa Clara, Calif.). The rice array contains over 23,000 genes (approximately 18,000 unique genes) or roughly 50% of the rice genome and is similar to the Arabidopsis GeneChip® (Affymetrix) with the exception that the 16 oligonucleotide probe sets do not contain mismatch probe sets. The level of expression is therefore determined by internal software that analyzes the intensity level of the 16 probe sets for each gene. The highest and lowest probes are removed if they do not fit into a set of predefined statistical criteria and the remaining sets are averaged to give an expression value. The final expression values are normalized by software, as described below. The advantages of a gene chip in such an analysis include a global gene expression analysis, quantitative results, a highly reproducible system, and a higher sensitivity than Northern blot analyses.

The GeneChip® Rice Genome Array was used to identify clusters of genes that were coordinately induced in response to various stress conditions. The GeneChip® Rice Genome Array contains probes synthesized in situ and is designed to measure temporal and spatial gene expression of approximately 18,000 genes which covers approximately 40-50% of the genome. The Affymetrix GeneChip® array was used to define transcription factor genes/pathways affected by various abiotic stresses and to define which are uniquely regulated by one stress and those that respond to multiple stress, and to identify candidate nucleotide sequences for screening for insertional mutants. Of the transcription factor genes represented on the Affymetrix GeneChip® array, certain nucleotide sequences showed at least a 2-fold change in expression in at least one sample, relative to no-treatment controls. Of those nucleotide sequences that were regulated only by cold stress, some were regulated only by drought stress and some were regulated only by saline stress.

The following describes in more detail how the experiments were done. Transcriptional profiling was performed by hybridizing fluorescence labeled cRNA with the oligonucleotides probes on the chip, washing, and scanning. Each gene is represented on the chip by about sixteen oligonucleotides (25-mers). Expression level is related to fluorescence intensity. Starting material contained 1 to 10 μg total RNA; detection specificity was about 1:10⁶; approximately a 2-fold change was detectable, with less than 2% false positive; the dynamic range was approximately 500×. Nucleotide sequences having up to 70% to 80% identity could be discriminated using this system.

3.1 GeneChip Standard Protocol

Quantitation of Total RNA

Total RNA from plant tissue is extracted and quantified.
1. Quantify total RNA using GeneQuant
   1OD₂₆₀=40 mg RNA/ml; A260/A280=1.9 to about 2.1
2. Run gel to check the integrity and purity of the extracted RNA
   Synthesis of Double-Stranded cDNA

Gibco/BRL SuperScript Choice System for cDNA Synthesis (Cat#1B090-019) was employed to prepare cDNAs. T7-(dT)₂₄ oligonucleotides were prepared and purified by HPLC. (5′ -GGCCAGTGAATTGTAATACGACTCACTATAGGGAGGCGG-(dT)₂₄-3′ SEQ ID NO:502).

Step 1. Primer Hybridization:
Incubate at 70° C. for 10 minutes
Quick spin and put on ice briefly

Step 2. Temperature Adjustment:
Incubate at 42° C. for 2 minutes

Step 3. First Strand Synthesis:
DEPC-water—1μl
RNA (10 μg final)—10 μl
T7=(dT)₂₄ Primer (100 pmol final)—1 μl pmol
5× 1st strand cDNA buffer—4 μl
0.1M DTT (10 mM final)—2 μl
10 mM dNTP mix (500 μM final)—1 μl
Superscript II RT 200 U/μl—1 μl
Total of 20 μl
Mix well
Incubate at 42° C. for 1 hour

Step 4. Second Strand Synthesis:
Place reactions on ice, quick spin
DEPC-water—91 μl
5× 2nd strand cDNA buffer—30 μl
mM dNTP mix (250 mM final)—3 μl
E. coli DNA ligase (10 U/μl)—1 μl
E. coli DNA polymerase 1-10 U/μl—4 μl
RnaseH 2 U/μl —1 μl
T4 DNA polymerase 5 U/μl—2 μl
0.5 M EDTA (0.5 M final)—10 μl
Total 162 μl
Mix/spin down/incubate 16° C. for 2 hours

Step 5. Completing the Reaction:
Incubate at 16° C. for 5 minutes
   Purification of Double Stranded cDNA

1. Centrifuge PLG (Phase Lock Gel, Eppendorf 5 Prime, Inc., PI-188233) at 14,000×, transfer 162 μl of cDNA to PLG

2. Add 162 μl of Phenol:Chloroform:Isoamyl alcohol (pH 8.0), centrifuge 2 minutes

3. Transfer the supernatant to a fresh 1.5 ml tube, add

| | |
|---|---|
| Glycogen (5 mg/ml) | 2 |
| 0.5 M NH₄OAC(0.75 × Vol) | 120 |
| ETOH (2.5 × Vol, −20 C) | 400 |

4. Mix well and centrifuge at 14,000 × for 20 minutes

5. Remove supernatant, add 0.5 ml 80% EtOH (−20° C.)

6. Centrifuge for 5 minutes, air dry or by speed vac for 5-10 minutes

7. Add 44 μl DEPC H₂O

Analyze of quantity and size distribution of cDNA

Run a gel using 1 μl of the double-stranded synthesis product

Synthesis of Biotinylated cRNA

(use Enzo BioArray High Yield RNA Transcript Labeling Kit Cat#900182)

| | |
|---|---|
| Purified cDNA | 22 μl |
| 10X Hy buffer | 4 μl |
| 10X biotin ribonucleotides | 4 μl |
| 10X DTT | 4 μl |
| 10X Rnase inhibitor mix | 4 μl |
| 20X T7 RNA polymerase | 2 μl |
| Total | 40 μl |

Centrifuge 5 seconds, and incubate for 4 hours at 37 EC

Gently mix every 30-45 minutes

Purification and Quantification of cRNA

(use Qiagen Rneasy Mini kit Cat# 74103)

Determine concentration and dilute to 1 μg/μl concentration

Fragmentation of cRNA

| | |
|---|---|
| cRNA (1 μg/μl) | 15 μl |
| 5X Fragmentation Buffer* | 6 μl |
| DEPC H₂O | 9 μl |
| | 30 μl |

*5× Fragmentation Buffer

| | | |
|---|---|---|
| IM Tris (pH8.1) | 4.0 | ml |
| MgOAc | 0.64 | g |
| KOAC | 0.98 | g |
| DEPC H₂O | | |
| Total | 20 | ml |
| Filter Sterilize | | |

Array Wash and Staining

Stringent Wash Buffer**

Non-Stringent Wash Buffer***

SAPE Stain****

Antibody Stain*****

Wash on fluidics station using the appropriate antibody amplification protocol
**Stringent Buffer: 12× MES 83.3 ml, 5 M NaCl 5.2 ml, 10% Tween 1.0 ml, H₂O 910 ml,

Filter Sterilize
***Non-Stringent Buffer: 20×SSPE 300 ml, 10% Tween 1.0 ml, H₂O 698 ml, Filter Sterilize, Antifoam 1.0.
****SAPE stain: 2× Stain Buffer 600 μl, BSA 48 μl, SAPE 12 μl, H₂O 540 μl.
*****Antibody Stain: 2× Stain Buffer 300 μl, H₂O 266.4 μl, BSA 24 μl, Goat IgG 6 μl,

Biotinylated Ab 3.6 μl

Image Analysis and Data Mining

1. Two text files are included in the analysis:
a. One with Absolute analysis: giving the status of each gene, either absent or present in the samples
b. The other with Comparison analysis: comparing gene expression levels between two samples
   3.2 Growth Conditions

Rice plants were grown for 6 weeks in convirons in plastic pots filled with sand. The conditions of the conviron are 12 h/12 h light/dark, 25° C., ˜50% RH and light intensity at 300 μEi. The plants were fertilized three times per week with one-half-strength Hoagland Solution containing 25 μM KH₂PO₄.

3.3 Abiotic Stress Treatment

Six weeks after placing the rice plants in convirons, stresses were applied as follows:
Control—no treatment;
Drought=25% PolyEthyleneGlycol (PEG) 8000 (PEG is a more controllable method for creating a water-deficit, the osmotic pressure from PEG will mimic the water-deficit experienced during drought)
Osmotic Stress=260.0 mM Mannitol (equivalent osmolarity of a 150.0 mM NaCl solution)
NaCl=150.0 mM
Cold=14° C. (the temperature at which pollen mother cell development is affected)
   The abiotic stress treatments was applied at time 0 and then at the same time of day on subsequent days (ie. Time 0, 24, 48 and 72 hours).
   3.4 Tissue Sampling

After the onset of treatment 3 time points were harvested, namely, 3 hr, 27 hr and 75 hr. Leaves and roots were harvested separately and the tissue flash-frozen in liquid nitrogen. These time points are set to be the exact same time of day at all 3 time points to eliminate the effects of circadian rhythms in gene expression. RNA was purified, and the samples were analyzed using the GeneChip® Rice Genome Array (Affymetrix, Santa Clara, Calif.) following the manufacturer's protocol.

3.5 Data Analysis

Raw fluorescence values as generated by Affymetrix software were processed as follows: the values were brought into Microsoft Excel and values of 25 or less were set to 25 (an empirically determined baseline as disclosed in Zhu and Wang, Plant Physiol. 124:1472-1476; 2000). The values from the stressed samples were then converted to fold change relative to control by dividing the values from the stressed samples by the values from the no-treatment control samples. Expression patterns that were altered at least 2-fold with respect to the control were selected. This method gave very robust results and resulted in a larger number of nucleotide sequences called as stress-regulated than previous methods had permitted.

Based on the profiles obtained following hybridization of nucleic acid molecules obtained from plant cells exposed to various stress conditions to the probes in the microarray, transcription factor genes that were altered at least two-fold in response to the stress conditions were identified (see Table 1).

Table 1, below, provides the SEQ ID NOs of those sequences identified by expression profiling which are at least 2× up- or down-regulated in response to an abiotic stress (salt, cold, osmotic or drought stress). Here, drought=25%, PolyEthyleneGlycol (PEG) 8000; osmotic stress=260.0 mM Mannitol; NaCl=150.0 mM; cold stress=14° C.

The results disclosed herein demonstrate that several polynucleotides, which were known to function as transcription factors, also are involved in the response of a plant cell to stress. The identification of stress-regulated genes as disclosed herein provides a means to identify stress-regulated regulatory elements present in rice nucleotide sequences, including consensus regulatory elements. Furthermore, the identification of the rice stress-regulated transcription factor genes provides a means to identify the corresponding homologs and orthologs in other plants, including commercially valuable food crops such as wheat, maize, soy, and barley, and ornamental plants.

TABLE 1

| SEQ ID NO. | Expression from Rice Chip Experiments |
|---|---|
| 1 | Induced by 75 hr drought treatment |
| 3 | Induced by 27 hr cold treatment |
| 4 | Repressed by 75 hr osmotic and cold |
| | treatment |
| 10 | Induced by 75 hr drought treatment |
| 11 | Induced by 75 hr drought treatment |
| 17 | Repressed by 75 hr drought treatment |
| 20 | Induced by 3 hr and 27 hr drought, osmotic, |
| | salt and cold treatment |
| 26 | Induced by 75 hr drought treatment and repressed |
| | by 27 hr osmotic and salt treatment |
| 27 | Induced by 75 hr drought treatment |
| 32 | Induced by 3 hr drought, osmotic and salt |
| | treatment |
| 42 | Induced by 3 hr and 27 hr cold treatment |
| 45 | Repressed by 3 hr osmotic, salt and 75 hr cold |
| | treatment |
| 46 | Repressed by 27 hr cold treatment |

### Example 4

### Rice Orthologs of Arabidopsis Transcription Factor Genes Identified by Reverse Genetics

Understanding the function of every gene is the major challenge in the age of completely sequenced eukaryotic genomes. Sequence homology can be helpful in identifying possible functions of many genes. However, reverse genetics, the process of identifying the function of a gene by obtaining and studying the phenotype of an individual containing a mutation in that gene, is another approach to identify the function of a gene.

Reverse genetics in Arabidopsis has been aided by the establishment of large publicly available collections of insertion mutants (Krysan et al., (1999) Plant Cell 11, 2283-2290; Tisser et al., (1999) Plant Cell 11, 1841-1852; Speulman et al., (1999) Plant Cell 11, 1853-1866; Parinov et al., (1999) Plant Cell 11, 2263-2270; Parinov and Sundaresan, 2000; Biotechnology 11, 157-161). Mutations in genes of interest are identified by screening the population by PCR amplification using primers derived from sequences near the insert border and the gene of interest to screen through large pools of individuals. Pools producing PCR products are confirmed by Southern hybridization and further deconvoluted into subpools until the individual is identified (Sussman et al., (2000) Plant Physiology 124, 1465-1467).

Recently, some groups have begun the process of sequencing insertion site flanking regions from individual plants in large insertion mutant populations, in effect prescreening a subset of lines for genomic insertion sites (Parinov et al., (1999) Plant Cell 11, 2263-2270; Tisser et al., (1999) Plant Cell 11, 1841-1852). The advantage to this approach is that the laborious and time-consuming process of PCR-based screening and deconvolution of pools is avoided.

A large database of insertion site flanking sequences from approximately 100,000 T-DNA mutagenized Arabidopsis plants of the Columbia ecotype (GARLIC lines) is prepared. T-DNA left border sequences from individual plants are amplified using a modified thermal asymmetric interlaced-polymerase chain reaction (TAIL-PCR) protocol (Liu et al., (1995). Plant J. 8, 457-463). Left border TAIL-PCR products are sequenced and assembled into a database that associates sequence tags with each of the approximately 100,000 plants in the mutant collection. Screening the collection for insertions in genes of interest involves a simple gene name or sequence BLAST query of the insertion site flanking sequence database, and search results point to individual lines. Insertions are confirmed using PCR.

Analysis of the GARLIC insert lines suggests that there are 76,856 insertions that localize to a subset of the genome representing coding regions and promoters of 22,880 genes. Of these, 49,231 insertions lie in the promoters of over 18,572 genes, and an additional 27,625 insertions are located within the coding regions of 13,612 genes. Approximately 25,000 T-DNA left border mTAIL-PCR products (25% of the total 102,765) do not have significant matches to the subset of the genome representing promoters and coding regions, and are therefore presumed to lie in noncoding and/or repetitive regions of the genome.

The Arabidopsis T-DNA GARLIC insertion collection is used to investigate the roles of certain transcription factor genes in the response to biotic and abiotic stresses. Target genes are chosen using a variety of criteria, including public reports of mutant phenotypes, RNA profiling experiments, and sequence similarity to transcription factor genes implicated in the abiotic or biotic stress response of the plant. Plant lines with insertions in genes of interest are then identified. Each T-DNA insertion line is represented by a seed lot collected from a plant that is hemizygous for a particular T-DNA insertion. Plants homozygous for insertions of interest are identified using a PCR assay. The seed produced by these plants is homozygous for the T-DNA insertion mutation of interest.

“Target gene” refers to a gene on a replicon that expresses the desired target coding sequence, functional RNA, or protein. The target gene is not essential for replicon replication. Additionally, target genes may comprise native non-viral genes inserted into a non-native organism, or chimeric genes, and will be under the control of suitable regulatory sequences. Thus, the regulatory sequences in the target gene may come from any source, including the virus. Target genes may include coding sequences that are either heterologous or homologous to the genes of a particular plant to be transformed. However, target genes do not include native viral genes. Typical target genes include, but are not limited to genes encoding a structural protein, a seed storage protein, a protein that conveys herbicide resistance, and a protein that conveys insect resistance. Proteins encoded by target genes are known as “foreign proteins”. The expression of a target gene in a plant will typically produce an altered plant trait.

Homozygous mutant plants are tested for altered responses to biotic or abiotic stresses. The genes interrupted in these mutants contribute to the observed phenotype. The genes interrupted in these mutants interfere with the normal timing and development of the plant flower.

Rice orthologs of the Arabidopsis genes affecting the plants response to biotic and/or abiotic stresses are identified by similarity searching of a rice database using the Double-Affine Smith-Waterman algorithm (BLASP with e values better than ⁻¹⁰)

### Example 5

### Cloning and Sequencing of Nucleic Acid Molecules from Rice

Genomic DNA: Plant genomic DNA samples can be isolated from frozen tissues, according to one of the three procedures, e.g., standard procedures described by Ausubel et al. (1995), a quick leaf prep described by Klimyuk et al. (1993), or using FTA paper (Life Technologies). For the latter procedure, a piece of leaf is excised from the plant, placed on top of the FTA paper and covered with a small piece of parafilm that serves as a barrier material to prevent contamination of the crushing device. In order to drive the sap and cells from the plant tissue into the FTA paper matrix for effective cell lysis and nucleic acid entrapment, a crushing device is used to mash the tissue into the FTA paper. The FTA paper is air dried for an hour. For analysis of DNA, the samples can be archived on the paper until analysis. Two mm punches are removed from the specimen area on the FTA paper using a 2 mm Harris Micro Punch™ and placed into PCR tubes. Two hundred (200) microliters of FTA purification reagent is added to the tube containing the punch and vortexed at low speed for 2 seconds. The tube is then incubated at room temperature for 5 minutes. The solution is removed with a pipette so as to repeat the wash one more time. Two hundred (200) microliters of TE (10 mM Tris, 0.1 mM EDTA, pH 8.0) is added and the wash is repeated two more times. The PCR mix is added directly to the punch for subsequent PCR reactions.

Cloning of Candidate cDNA: A candidate cDNA is amplified from total RNA isolated from rice tissue after reverse transcription using primers designed against the computationally predicted cDNA. Primers designed based on the genomic sequence can be used to PCR amplify the full-length cDNA (start to stop codon) from first strand cDNA prepared from rice cultivar Nipponbare tissue.

The Qiagen RNeasy kit (Qiagen, Hilden, Germany) is used for extraction of total RNA. The Superscript II kit (Invitrogen, Carlsbad, USA) is used for the reverse transcription reaction. PCR amplification of the candidate cDNA is carried out using the reverse primer sequence located at the translation start of the candidate gene in 5′-3′ direction. This is performed with high-fidelity Taq polymerase (Invitrogen, Carlsbad, USA).

The PCR fragment is then cloned into pCR2.1-TOPO (Invitrogen) or the pGEM-T easy vector (Promega Corporation, Madison, Wis., USA) per the manufacturer's instructions, and several individual clones are subjected to sequencing analysis.

DNA sequencing: DNA preps for 2-4 independent clones are miniprepped following the manufacturer's instructions (Qiagen). DNA is subjected to sequencing analysis using the BigDye™ Terminator Kit according to manufacturer's instructions (ABI). Sequencing makes use of primers designed to both strands of the predicted gene of interest. DNA sequencing is performed using standard dye-terminator sequencing procedures and automated sequencers (models 373 and 377; Applied Biosystems, Foster City, Calif.). All sequencing data are analyzed and assembled using the Phred/Phrap/Consed software package (University of Washington) to an error ratio equal to or less than 10⁻⁴ at the consensus sequence level.

The consensus sequence from the sequencing analysis is then to be validated as being intact and the correct gene in several ways. The coding region is checked for being full length (predicted start and stop codons present) and uninterrupted (no internal stop codons). Alignment with the gene prediction and BLAST analysis is used to ascertain that this is in fact the right gene.

The clones are sequenced to verify their correct amplification.

### Example 6

### Functional Analysis in Plants

A plant complementation assay can be used for the functional characterization of the transcription factor genes according to the invention.

Rice and Arabidopsis putative orthologue pairs are identified using BLAST comparisons, TFASTXY comparisons, and Double-Affine Smith-Waterman similarity searches. Constructs containing a rice cDNA or genomic clone inserted between the promoter and terminator of the Arabidopsis orthologue are generated using overlap PCR (Gene 77, 61-68 (1989)) and GATEWAY cloning (Life Technologies Invitrogen). For ease of cloning, rice cDNA clones are preferred to rice genomic clones. A three stage PCR strategy is used to make these constructs.

(1) In the first stage, primers are used to PCR amplify: (i) 2 Kb upstream of the translation start site of the Arabidopsis orthologue, (ii) the coding region or cDNA of the rice orthologue, and (iii) the 500 bp immediately downstream of the Arabidopsis orthogue's translation stop site. Primers are designed to incorporate onto their 5′ ends at least 16 bases of the 3′ end of the adjacent fragment, except in the case of the most distal primers which flank the gene construct (the forward primer of the promoter and the reverse primer of the terminator). The forward primer of the promoters contains on their 5′ ends partial AttB1 sites, and the reverse primer of the terminators contains on their 5′ ends partial AttB2 sites, for Gateway cloning.

(2) In the second stage, overlap PCR is used to join either the promoter and the coding region, or the coding region and the terminator.

(3) In the third stage either the promoter-coding region product can be joined to the terminator or the coding region-terminator product can be joined to the promoter, using overlap PCR and amplification with fulll Att site-containing primers, to link all three fragments, and put full Att sites at the construct termini.

The fused three-fragment piece flanked by Gateway cloning sites are introduced into the LTI donor vector pDONR201 using the BP clonase reaction, for confirmation by sequencing. Confirmed sequenced constructs are introduced into a binary vector containing Gateway cloning sites, using the LR clonase reaction such as, for example, pAS200.

The pAS200 vector was created by inserting the Gateway cloning cassette RfA into the Acc65I site of pNOV3510.

pNOV3510 was created by ligation of inverted pNOV2114 VSI binary into pCTK7-PTX5′AtPPONOS.

pNOV2114 was created by insertion of virGN54D (Pazour et al. 1992, J. Bacteriol. 174:4169-4174) from pAD1289 (Hansen et al. 1994, PNAS 91:7603-7607) into pHiNK085.

pHiNK085 was created by deleting the 35S:PMI cassette and M13 ori in pVictorHiNK.

pPVictorHiNK was created by modifying the T-DNA of pVictor (described in WO 97/04112) to delete M13 derived sequences and to improve its cloning versatility by introducing the BIGLINK polylinker.

The sequence of the pVictor HiNK vector is disclosed in SEQ ID NO: 5 in WO 00/6837, which is incorporated herein by reference. The pVictorHiNK vector contains the following constituents that are of functional importance:
The origin of replication (ORI) functional in Agrobacterium is derived from the Pseudomonas aeruginosa plasmid pVS1 (Itoh et al. 1984. Plasmid 11: 206-220; Itoh and Haas, 1985. Gene 36: 27-36). The pVS1 ORI is only functional in Agrobacterium and can be mobilised by the helper plasmid pRK2013 from E.coli into A. tumefaciens by means of a triparental mating procedure (Ditta et al., 1980. Proc. Natl. Acad. Sci USA 77: 7347-7351).
The ColE1 origin of replication functional in E. coli is derived from pUC19 (Yannisch-Perron et al., 1985. Gene 33: 103-119).
The bacterial resistance to spectinomycin and streptomycin encoded by a 0.93 kb fragment from transposon Tn7 (Fling et al., 1985. Nucl. Acids Res. 13: 7095) functions as selectable marker for maintenance of the vector in E. coli and Agrobacterium. The gene is fused to the tac promoter for efficient bacterial expression (Amman et al., 1983. Gene 25: 167-178).
The right and left T-DNA border fragments of 1.9 kb and 0.9 kb that comprise the 24 bp border repeats, have been derived from the Ti-plasmid of the nopaline type Agrobacterium tumefaciens strains pTiT37 (Yadav et al., 1982. Proc. Natl. Acad. Sci. USA. 79: 6322-6326).

The plasmid is introduced into Agrobacterium tumefaciens GV3101pMP90 by electroporation. The positive bacterial transformants are selected on LB medium containing 50 μg/μl kanamycin and 25 μg/μl gentamycin. Plants are transformed by standard methodology (e.g., by dipping flowers into a solution containing the Agrobacterium) except that 0.02% Silwet-77 (Lehle Seeds, Round Rock, Tex.) is added to the bacterial suspension and the vacuum step omitted. Five hundred (500) mg of seeds are planted per 2 ft² flat of soil and plant transformants are selected by spraying with the herbicide formulated BASTA (2 ml of Finale, AgrEvo Environmental Health, Montvale, N.J., is added to 498 ml water) once every two days, for a week.

### Example 7

### Vector Construction for Overexpression and Gene “Knockout” Experiments

Overexpression

Vectors used for expression of full-length “candidate genes” of interest in plants (overexpression) are designed to overexpress the protein of interest and are of two general types, biolistic and binary, depending on the plant transformation method to be used.

For biolistic transformation (biolistic vectors), the requirements are as follows:
1. a backbone with a bacterial selectable marker (typically, an antibiotic resistance gene) and origin of replication functional in Escherichia coli (E. coli; eg. ColE1), and
2. a plant-specific portion consisting of:
   a. a gene expression cassette consisting of a promoter (eg. ZmUBlint MOD), the gene of interest (typically, a full-length cDNA) and a transcriptional terminator (eg. Agrobacterium tumefaciens nos terminator);
   b. a plant selectable marker cassette, consisting of a promoter (eg. rice Act1D-BV MOD), selectable marker gene (eg. phosphomannose isomerase, PMI) and transcriptional terminator (eg. CaMV terminator).

Vectors designed for transformation by Agrobacterium tumefaciens (A. tumefaciens; binary vectors) consist of:
1. a backbone with a bacterial selectable marker functional in both E. coli and A. tumefaciens (eg. spectinomycin resistance mediated by the aadA gene) and two origins of replication, functional in each of aforementioned bacterial hosts, plus the A. tumefaciens virG gene;
2. a plant-specific portion as described for biolistic vectors above, except in this instance this portion is flanked by A. tumefaciens right and left border sequences which mediate transfer of the DNA flanked by these two sequences to the plant.
   Knock Out Vectors

Vectors designed for reducing or abolishing expression of a single gene or of a family or related genes (knockout vectors) are also of two general types corresponding to the methodology used to downregulate gene expression: antisense or double-stranded RNA interference (dsRNAi).

(a) Anti-Sense

For antisense vectors, a full-length or partial gene fragment (typically, a portion of the cDNA) can be used in the same vectors described for full-length expression, as part of the gene expression cassette. For antisense-mediated down-regulation of gene expression, the coding region of the gene or gene fragment will be in the opposite orientation relative to the promoter; thus, mRNA will be made from the non-coding (antisense) strand in planta.

(b) dsRNAi

For dsRNAi vectors, a partial gene fragment (typically, 300 to 500 basepairs long) is used in the gene expression cassette, and is expressed in both the sense and antisense orientations, separated by a spacer region (typically, a plant intron, eg. the OsSH1 intron 1, or a selectable marker, eg. conferring kanamycin resistance). Vectors of this type are designed to form a double-stranded mRNA stem, resulting from the basepairing of the two complementary gene fragments in planta.

Biolistic or binary vectors designed for overexpression or knockout can vary in a number of different ways, including eg. the selectable markers used in plant and bacteria, the transcriptional terminators used in the gene expression and plant selectable marker cassettes, and the methodologies used for cloning in gene or gene fragments of interest (typically, conventional restriction enzyme-mediated or Gateway™ recombinase-based cloning). An important variant is the nature of the gene expression cassette promoter driving expression of the gene or gene fragment of interest in most tissues of the plants (constitutive, eg. ZmUBIint MOD), in specific plant tissues (eg. maize ADP-gpp for endosperm-specific expression), or in an inducible fashion (eg. GAL4bsBz1 for estradiol-inducible expression in lines constitutively expressing the cognate transcriptional activator for this promoter).

### Example 8

### Insertion of a “candidate gene” Involved in Flower Timing into a Plant Expression Vector

A validated rice cDNA clone in pCR2.1-TOPO or the pGEM-T easy vector is subcloned using conventional restriction enzyme-based cloning into a vector, downstream of the maize ubiquitin promoter and intron, and upstream of the Agrobacterium tumefaciens nos 3′ end transcriptional terminator. The resultant gene expression cassette (promoter, “candidate gene” and terminator) is further subcloned, using conventional restriction enzyme-based cloning, into the pNOV2117 binary vector (Negrotto et al (2000) Plant Cell Reports 19, 798-803; plasmid pNOV117 discosed in this article corresponds to pNOV2117 described herein), generating pNOVCAND.

The pNOVCAND binary vector is designed for transformation and over-expression of the “candidate gene” in monocots. It consists of a binary backbone containing the sequences necessary for selection and growth in Escherichia coli DH-5alpha (Invitrogen) and Agrobacterium tumefaciens LBA4404 (pAL4404; pSB1), including the bacterial spectinomycin antibiotic resistance aadA gene from E. coli transposon Tn7, origins of replication for E. coli (ColE1) and A. tumefaciens (VS1), and the A. tumefaciens virG gene. In addition to the binary backbone, which is identical to that of pNOV2114 described herein previously (see Example 6 above) pNOV2117 contains the T-DNA portion flanked by the right and left border sequences, and including the Positech™ (Syngenta) plant selectable marker (WO 94/20627) and the “candidate gene” gene expression cassette. The Positech™ plant selectable marker confers resistance to mannose and in this instance consists of the maize ubiquitin promoter driving expression of the PMI (phosphomannose isomerase) gene, followed by the cauliflower mosaic virus transcriptional terminator.

Plasmid pNOV2117 is introduced into Agrobacterium tumefaciens LBA4404 (pAL4404; pSB1) by electroporation. Plasmid pAL4404 is a disarmed helper plasmid Ooms et al., Plasmid 7, 15-29 (1982). Plasmid pSB1 is a plasmid with a wide host range that contains a region of homology to pNOV2117 and a 15.2 kb KpnI fragment from the virulence region of pTiBo542 (Ishida et al., Nat Biotechnol 14, 745-750 (1996). Introduction of plasmid pNOV2117 into Agrobacterium strain LBA4404 results in a co-integration of pNOV2117 and pSB1. Alternatively, plasmid pCIB7613, which contains the hygromycin phosphotransferase (hpt) gene, Gritz and Davies, Gene 25, 179-188 (1983), as a selectable marker, may be employed for transformation.

Plasmid pCIB7613 (see WO 98/06860, incorporated herein by reference in its entirety) is selected for rice transformation. In pCIB7613, the transcription of the nucleic acid sequence coding hygromycin-phosphotransferase (HYG gene) is driven by the corn ubiquitin promoter (ZmUbi) and enhanced by corn ubiquitin intron 1. The 3′polyadenylation signal is provided by NOS 3′ nontranslated region.

Other useful plasmids include pNADII002 (GAL4-ER-VP16), which contains the yeast GAL4 DNA Binding domain (Keegan et al., Science, 231:699 (1986)), the mammalian estrogen receptor ligand binding domain (Greene et al., Science, 231:1150 (1986)) and the transcriptional activation domain of the HSV VP16 protein (Triezenberg et al.,1988). Both hpt and GAL4-ER-VP16 are constitutively expressed using the maize Ubiquitin promoter, and pSGCDL1 (GAL4BS Bz1 Luciferase), which carries the firefly luciferase reporter gene under control of a minimal maize Bronze1 (Bz1) promoter with 10 upstream synthetic GAL4 binding sites. All constructs use termination signals from the nopaline synthase gene.

### Example 9

### Rice Transformation

pNOVCAND is transformed into a rice cultivar (Kaybonnet) using Agrobacterium-mediated transformation, and mannose-resistant calli are selected and regenerated.

Agrobacterium is grown on YPC solid plates for 2-3 days prior to experiment initiation. Agrobacterial colonies are suspended in liquid MS media to an OD of 0.2 at λ600 nm. Acetosyringone is added to the agrobacterial suspension to a concentration of 200 μM and agro is induced for 30 min.

Three-week-old calli which are induced from the scutellum of mature seeds in the N6 medium (Chu, C. C. et al., Sci, Sin., 18, 659-668(1975)) are incubated in the agrobacterium solution in a 100×25 petri plate for 30 minutes with occasional shaking. The solution is then removed with a pipet and the callus transfered to a MSAs medium which is overlayed with sterile filter paper.

Co-Cultivation is continued for 2 days in the dark at 22° C.

Calli are then placed on MS-Timetin plates for 1 week. After that they are tranfered to PAA+mannose selection media for 3 weeks.

Growing calli (putative events) are picked and transfered to PAA+mannose media and cultivated for 2 weeks in light.

Colonies are tranfered to MS20SorbKinTim regeneration media in plates for 2 weeks in light. Small plantlets are transferred to MS20SorbKinTim regeneration media in GA7 containers. When they reach the lid, they are transfered to soil in the greenhouse.

Expression of the “candidate gene” in transgenic T₀ plants is analyzed. Additional rice cultivars, such as but not limited to, Nipponbare, Taipei 309 and Fuzisaka 2 are also transformed and assayed for expression of the “candidate gene” product and enhanced protein expression.

### Example 10

### Method of Modifying the Gene Frequency

The invention further provides a method of modifying the frequency of a gene in a plant population, including the steps of: identifying an SSR within a coding region of a gene; screening a plurality of plants using the SSR as a marker to determine the presence or absence of the gene in an individual plant; selecting at least one individual plant for breeding based on the presence or absence of the gene; and breeding at least one plant thus selected to produce a population of plants having a modified frequency of the gene. The identification of the SSR within the coding region of a gene can be accomplished based on sequence similarity between the nucleic acid molecules of the invention and the region within the gene of interest flanking the SSR.

### Example 11

### Chromosomal Markers to Identify the Location of a Nucleic Acid Sequence

The sequences of the present invention can also be used for SSR mapping. SSR mapping in rice has been described by Miyao et al. (DNA Res 3:233 (1996)) and Yang et al. (Mol Gen Genet 245:187 (1994)), and in maize by Ahn et al. (Mol Gen Genet 241:483 (1993)). SSR mapping can be achieved using various methods. In one instance, polymorphisms are identified when sequence specific probes flanking an SSR contained within a sequence are made and used in polymerase chain reaction (PCR) assays with template DNA from two or more individuals or, in plants, near isogenic lines. A change in the number of tandem repeats between the SSR-flanking sequence produces differently sized fragments (U.S. Pat. No. 5,766,847). Alternatively, polymorphisms can be identified by using the PCR fragment produced from the SSR-flanking sequence specific primer reaction as a probe against Southern blots representing different individuals (Refseth et al., Electrophoresis 18:1519 (1997)). Rice SSRs can be used to map a molecular marker closely linked to functional gene, as described by Akagi et al. (Genome 39:205 (1996)).

The sequences of the present invention can be used to identify and develop a variety of microsatellite markers, including the SSRs described above, as genetic markers for comparative analysis and mapping of genomes.

Many of the polynucleotides used contain at least 3 consecutive di-, tri- or tetranucleotide repeat units in their coding region that can potentially be developed into SSR markers. Trinucleotide motifs that can be commonly found in the coding regions of said polynucleotides and easily identified by screening the polynucleotides sequences for said motifs are, for example: CGG; GCC, CGC, GGC, etc. Once such a repeat unit has been found, primers can be designed which are complementary to the region flanking the repeat unit and used in any of the methods described below.

Sequences of the present invention can also be used in a variation of the SSR technique known as inter-SSR (ISSR), which uses microsatellite oligonucleotides as primers to amplify genomic segments different from the repeat region itself (Zietkiewicz et al., Genomics 20:176 (1994)). ISSR employs oligonucleotides based on a simple sequence repeat anchored or not at their 5′ - or 3′-end by two to four arbitrarily chosen nucleotides, which triggers site-specific annealing and initiates PCR amplification of genomic segments which are flanked by inversely orientated and closely spaced repeat sequences. In one embodiment of the present invention, microsatellite markers as disclosed herein, or substantially similar sequences or allelic variants thereof, may be used to detect the appearance or disappearance of markers indicating genomic instability as described by Leroy et al. (Electron. J Biotechnol, 3(2), at http://www.ejb.org (2000)), where alteration of a fingerprinting pattern indicated loss of a marker corresponding to a part of a gene involved in the regulation of cell proliferation. Microsatellite markers are useful for detecting genomic alterations such as the change observed by Leroy et al. (Electron. J Biotechnol, 3(2), supra (2000)) which appeared to be the consequence of microsatellite instability at the primer binding site or modification of the region between the microsatellites, and illustrated somaclonal variation leading to genomic instability. Consequently, sequences of the present invention are useful for detecting genomic alterations involved in somaclonal variation, which is an important source of new phenotypes.

In addition, because the genomes of closely related species are largely syntenic (that is, they display the same ordering of genes within the genome), these maps can be used to isolate novel alleles from wild relatives of crop species by positional cloning strategies. This shared synteny is very powerful for using genetic maps from one species to map genes in another. For example, a gene mapped in rice provides information for the gene location in maize and wheat.

### Example 12

### Quantitative Trait Linked Breeding

Various types of maps can be used with the sequences of the invention to identify Quantitative Trait Loci (QTLs) for a variety of uses, including marker-assisted breeding. Many important crop traits are quantitative traits and result from the combined interactions of several genes. These genes reside at different loci in the genome, often on different chromosomes, and generally exhibit multiple alleles at each locus. Developing markers, tools, and methods to identify and isolate the QTLs involved in a trait, enables marker-assisted breeding to enhance desirable traits or suppress undesirable traits. The sequences disclosed herein can be used as markers for QTLs to assist marker-assisted breeding. The sequences of the invention can be used to identify QTLs and isolate alleles as described by Li et al. in a study of QTLs involved in resistance to a pathogen of rice. (Li et al., Mol Gen Genet 261:58 (1999)). In addition to isolating QTL alleles in rice, other cereals, and other monocot and dicot crop species, the sequences of the invention can also be used to isolate alleles from the corresponding QTL(s) of wild relatives. Transgenic plants having various combinations of QTL alleles can then be created and the effects of the combinations measured. Once an ideal allele combination has been identified, crop improvement can be accomplished either through biotechnological means or by directed conventional breeding programs. (Flowers et al., J Exp Bot 51:99 (2000); Tanksley and McCouch, Science 277:1063 (1997)).

### Example 13

### Marker-Assisted Breeding

Markers or genes associated with specific desirable or undesirable traits are known and used in marker assisted breeding programs. It is particularly beneficial to be able to screen large numbers of markers and large numbers of candidate parental plants or progeny plants. The methods of the invention allow high volume, multiplex screening for numerous markers from numerous individuals simultaneously.

Markers or genes associated with specific desirable or undesirable traits are known and used in marker assisted breeding programs. It is particularly beneficial to be able to screen large numbers of markers and large numbers of candidate parental plants or progeny plants. The methods of the invention allow high volume, multiplex screening for numerous markers from numerous individuals simultaneously.

A multiplex assay is designed providing SSRs specific to each of the markers of interest. The SSRs are linked to different classes of beads. All of the relevant markers may be expressed genes, so RNA or cDNA techniques are appropriate. RNA is extracted from root tissue of 1000 different individual plants and hybridized in parallel reactions with the different classes of beads. Each class of beads is analyzed for each sample using a microfluidics analyzer. For the classes of beads corresponding to qualitative traits, qualitative measures of presence or absence of the target gene are recorded. For the classes of beads corresponding to quantitative traits, quantitative measures of gene activity are recorded. Individuals showing activity of all of the qualitative genes and highest expression levels of the quantitative traits are selected for further breeding steps. In procedures wherein no individuals have desirable results for all the measured genes, individuals having the most desirable, and fewest undesirable, results are selected for further breeding steps. In either case, progeny are screened to further select for homozygotes with high quantitative levels of expression of the quantitative traits.

### Example 14

### Promoter Analysis

The gene chip experiment described above are designed to uncover genes that are expressed in seed tissue during grain filling. Candidate promoters are identified based upon the expression profiles of the associated transcripts.

Candidate promoters are obtained by PCR and fused to a GUS reporter gene containing an intron. Both histochemical and fluormetric GUS assays are carried out on stably transformed rice and maize plants and GUS activity is detected in the transformants.

Further, transient assays with the promoter::GUS constructs are carried out in rice embryogenic callus and GUS activity is detected by histochemical staining according the protocol described below.

Construction of Binary Promoter::Reporter Plasmids

To construct a binary promoter::reporter plasmid for rice transformation a vector containing a promoter of interest (i.e., the DNA sequence 5′ of the initiation codon for the gene of interest) is used, which results from recombination in a BP reaction between a PCR product using the promoter of interest as a template and pDONR201™, producing an entry vector. The regulatory/promoter sequence is fused to the GUS reporter gene (Jefferson et al, 1987) by recombination using GATEWAY™ Technology according to manufacturers protocol as described in the Instruction Manual (GATEWAY™ Cloning Technology, GIBCO BRL, Rockville, Md. http://www.lifetech.com/).

Briefly, the Gateway Gus-intron-Gus (GIG)/NOS expression cassette is ligated into pNOV2117 binary vector in 5′ to 3′ orientation. The 4.1 kB expression cassette is ligated into the Kpn-I site of pNOV2117, then clones are screened for orientation to obtain pNOV2346, a GATEWAY™ adapted binary destination vector.

The promoter fragment in the entry vector is recombined via the LR reaction with the binary destination vector containing the GUS coding region with an intron that has an attR site 5′ to the GUS reporter, producing a binary vector with a promoter fused to the GUS reporter (pNOVCANDProm). The orientation of the inserted fragment is maintained by the att sequences and the final construct is verified by sequencing. The construct is then transformed into Agrobacterium tumefaciens strains by electroporation as described herein previously.

### Example 15

### Transient Expression Analysis of Candidate Promoters in Rice Embryogenic Callus

Materials:

Embryogenic rice callus (Kaybonett cultivar)
LBA 4404 Agrobacterium strains
KCMS liquid media for re-suspending bacterial pellet
200 mM stock (40 mg/ml) Acetosyringone
Sterile filter paper discs (8.5 mm in diameter)
LB spec liquid culture
MS-CIM media plates
MS-AS plates (co-cultivation plates)
MS-Tim plates (recovery plates)
Gus staining solution
   Methods:
   Induction of Embryogenic Callus:

1. Sterilize mature Kaybonett rice seeds in 40% ultra Clorox, 1 drop Tween 20, for 40 min.

2. Rinse with sterile water and plate on MS-CIM media (12 seeds/plate)

3. Grow in dark for four weeks.

4. Isolate embryogenic calli from scutellum to MS-CIM

5. Let grow in dark 8 days before use for transformation

Agrobacterium preparation and induction:

1. Start 6 mL shaking cultures of LBA4404 Agrobacterium strains harboring rice promoter binary plasmids.

2. Grow the cultures at room temperature for 48 hrs in the rotary shaker.

3. Spin down the cultures at 8'000 rpm at 4° C. and re-suspend bacterial pellets in 10 ml of KCMS media supplemented with 100□M Acetosyringone.

4. Place in the shaker at room temp for 1 hr for induction of Agrobacterium virulence genes.

5. In a sterile hood dilute Agrobacterium cultures 1:3 in KSMS media and transfer diluted cultures into deep petri dishes.

Inoculation of Plant Material and Staining:

6. In a sterile hood transfer embryogenic callus into diluted Agrobacerium solution and incubate for 30 minutes.

7. In a sterile hood blot callus tissue on sterile filter paper and transfer on MS-AS plates.

8. Co-culture plates in 22° C. growth chamber in the dark for two days.

9. In a sterile hood transfer callus tissue to MS-Tim plates for the tissue recovery (the presence of Timentin will prevent Agrobacterium growth).

10. Incubate tissue on MS-Tim media for two days at 22° C. in the dark.

11. Remove callus tissue from the plates and stain for 48 hrs. in GUS staining solution.

12. De-stain tissue in 70% EtOH for 24 hours.

Recipies:

KCMS Media (liquid), pH to 5.5

100 ml/l MS Major Salts, 10 ml/l MS Minor Salts, 5 ml/l MS iron stock, 0.5M K₂HPO₄, 0.1 mg/n Myo-Inositol,

1.3 μg/ml Thiamine, 0.2 g/ml 2,4-D (1 mg/ml), 0.1 g/ml Kinetin, 3% Sucrose, 100 M Acetosyringone

MS-CIM Media, pH 5.8

MS Basal salt (4.3 g/L), B5 Vitamins (200×) (5 m/L), 2% Sucrose (20 g/L), Proline (500 mg/L), Glutamine (500 mg/L), Casein Hydrolysate (300 mg/L), 2□g/ml 2,4-D, Phytagel (3 g/L)

MS-As Medium, pH 5.8

MS Basal salt (4.3 g/L), B5 Vitamins (200×) (5 m/L), 2% Sucrose (20 g/L), Proline (500 mg/L), Glutamine (500 mg/L), Casein Hydrolysate (300 mg/L), 2 g/ml 2,4-D, Phytagel (3 g/L), 200 M Acetosyringone

MS-Tim Media, pH 5.8

MS Basal salt (4.3 g/L), B5 Vitamins (200×) (5 m/L), 2% Sucrose (20 g/L), Proline (500 mg/L), Glutamine (500 mg/L), Casein Hydrolysate (300 mg/L), 2 g/ml 2,4-D, Phytagel (3 g/L), 400 mg/l Timentin

Gus Staining Solution, pH 7

0.3M Mannitol; 0.02M EDTA, pH=7.0; 0.04 NaH₂PO₄; 1 mM x-gluc

### CONCLUSION

In light of the detailed description of the invention and the examples presented above, it can be appreciated that the several aspects of the invention are achieved.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventor does not intend to be bound by those conclusions and functions, but puts them forth only as possible explanations. It is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations within the spirit and scope of the following statements of the invention.

### REFERENCES

Altschul et al., J. Mol. Biol. 215:403 (1990).
Altschul et al., Nucleic Acids Res. 25:3389 (1997).
An et al., EMBO J. 4:277 (1985).
Ahn et al., Mol Gen Genet 241:483 (1993).
Akagi et al., Genome 39:205 (1996).
Amman et al., Gene 25: 167-178 (1983).
Auch & Reth et al. Nuc. Acids Res., 18:6743 (1990).
Ausubel et al., Current Protocols in Molecular Biology, Greene Publishing (1992) with updates.
Aoyama et al., N—H Plant Journal, 11:605 (1997).
Arabidopsis Genome Initiative, Nature, 409:796 (2001).
Ballas et al., Nucleic Acids Res. 17:7891 (1989).
Bansal et al., Proc. Natl. Acad. Sci. USA, 89:3654 (1992).
Batzer et al., Nucleic Acid Res., 19:5081 (1991).
Beals et al., Plant Cell, 9:1527 (1997).
Belanger et al., Genetics, 129:863 (1991).
Bevan et al., Nature, 304:184 (1983).
Blochinger & Diggelmann, Mol Cell Biol, 4:2929.
Borden, K. L. J. Mol. Biol. 295, 1103-1112. (2000).
Bird et al., Plant Molecular Biology, 11:651 (1988).
Bouchez et al., EMBO J., 8:4197 (1989).
Bourouis et al., EMBO J., 2:1099 (1983).
Bowler et al., Ann. Rev. Plant Physiol., 43:83 (1992).
Bourouis et al., EMBO J., 2:1099 (1983).
Byrne et al. Plant Cell Tissue and Organ Culture, 8:3 (1987).
Callis et al., Genes Devel., 1:1183, 1987
Capili, A. D., et al., Embo J. 20, 165-177(2001).
Coe et al., In: Corn and Corn Improvement, Sprague et al. (eds.) pp. 81-258, (1988)
Chu, C. C. et al., Sci, Sin., 18, 659-668(1975)
Cutler et al., J. Plant Physiol., 135:351,(1989)
Campbell and Gowri, Plant Physiol., 92:1 (1990).
Chandler et al., Plant Cell, 1:1175 (1989).
Chee et al., Science, 274:610-614 (1996).
Christou et al., Biotechnology, 9:957 (1991).
Christou et al., Plant Physiol., 87:671 (1988).
Cordero et al., Plant J., 6:141 (1994).
Corpet et al., Nucleic Acids Res. 16:10881 (1988).
Crameri et al., Nature Biotech., 15:436 (1997).
Crameri et al., Nature, 391:288 (1998).
Crossway et al., BioTechniques, 4:320 (1986).
Czako et al., Mol. Gen. Genet. 23 5 (1), 33-40 (1992).
Datta et al., Bio/Technology, 8, 736 (1990).
Dayhoff et al., Atlas of Protein Sequence and Structure, Natl. Biomed. Res. Found., Washington, C.D. (1978).
De Blaere et al., Meth. Enzymol. 143:277 (1987).
Della-Cioppa et al., Plant Physiology, 84:965 (1987).
Dennis et al., Nucleic Acids Res., 12:3983 (1984).
De Block et al., Plant Physiol. 91:694, (1989)
De Block et al., EMBO J., 6:2513 (1987)
Dellaporta et al., Chromosome Structure and Function, pp. 263-282 1988
Diekman & Fischer, EMBO J., 7:3315, 1988
Ditta et al., Proc. Natl. Acad. Sci USA 77: 7347-7351 (1980).
Everett et al., Bio/Technology, 5:1201, 1987
Eisen, M. B., Spellman, P. T., Brown, P. O., and Botstein, D. (1998). Proc. Natl. Acad. Sci. USA 95, 14863-14868.
Ellis et al., EMBO J., 6:3203 (1987).
Elroy-Stein et al., PNAS USA, 86:6126 (1989).
Eulgem et al., Trends in Plant Sci. 5:199 (2000).
English, et al., Plant Cell 8:179 (19 Fling et al., Nucl. Acids Res. 13: 7095 (1985).
Flowers et al., J Exp Bot 51:99 (2000);
Fromm et al., Nature, 319:791, (1986)
Franken et al., EMBO J., 10:2605 (1991).
Fromm et al., Bio/Technology 8:833 (1990).
Gallie et al., Molecular Biology of RNA, 237 (1989).
Gallie et al., Nucl. Acids Res., 15:8693 (1987).
Gan et al., Science, 270 (5244), 1986-8) (1995).
Gatz Current Opinion in Biotechnology, 7:168 (1996).
Gatz, C., Annu. Rev. Plant Physiol. Plant Mol. Biol., 48:89 (1997).
Gordon-Kamm et al., Plant Cell, 2, 603 (1990).
Graham et al., Biochem. Biophys. Res. Comm., 101:1164 (1981).
Graham et al., J. Biol. Chem., 260:6555 (1985).
Graham et al., J. Biol. Chem., 260:6561 (1985)
Guerineau et al., Mol. Gen. Genet. 262:141 (1991).
Greene et al., Science, 231:1150 (1986))
Gritz and Davies, Gene 25, 179-188 (1983)
Gruber et al. (“Vectors for Plant Transformation, in Methods” in Plant Molecular Biology & Biotechnology, Glich et al., Eds. pp. 89-119, CRC Press, 1993).
Gupta et al., Proc. Natl. Acad. Sci, USA, 90:1629, 1993
Haines and Higgins (eds.), Nucleic Acid Hybridization, IRL Press, Oxford, U.K
Hansen et al., PNAS 91:7603-7607(1994).
Henikoff & Henikoff, Proc. Natl. Acad. Sci. USA, 89:10915 (1989).
Hiei et al., Plant J. 6:271 (1994).
Higgins et al., Gene 73:237 (1988).
Higgins et al., CABIOS 5:151 (1989).
Hinchee et al., Biotechnology, 6:915 (1988).
Hoekema, In: The Binary Plant Vector System. Offset-drukkerij Kanters B.V., Alblasserdam, Chapter V (1985).
Huang et al., CABIOS 8:155 (1992).
Hudspeth & Grula, Plant Molec. Biol., 12:579 (1989).
Huffman et al., J. Cell. Biochem., 17B: Abstract.
Ingelbrecht et al., Plant Cell, 1:671 (1989).
Innis et al., eds., PCR Protocols: A Guide to Methods and Applications (Academic Press, New York (1995).
Innis &Gelfand, eds., PCR Methods Manual,Academic Press, New York (1999).
Itoh et al., Plasmid 11: 206-220 (1984).
Itoh and Haas, Gene 36: 27-36 (1985).
Ikuta et al., Biotech., 8:241 1990
Ishida et al., Nat Biotechnol 14, 745-750 (1996)
Jefferson, EMBO J. 6:3901-3907, 1997
Jensen, R. B., et al. FEBS Lett. 436, 283-287(1998).
Jobling et al., Nature, 325:622 (1987).
John et al., Proc. Natl. Acad. Sci. USA 89(13):5769 (1992).
Joshi et al., Nucleic Acid Res. 15:9627 (1987).
Joshi, Nucl. Acid Res., 15, 6643 (1987).
Karlin and Altschul, Proc. Natl. Acad Sci. USA 87:2264 (1990).
Karlin and Altschul, Proc. Natl. Acad. Sci. USA 90:5873 (1993).
Keller et al., Genes Dev., 3:1639 (1989).
Klein et al., Bio/Technology, 6:559 (1988).
Klein et al., Nature (London) 327:70 (1987).
Klein et al., Plant Physiol., 91:440 (1988).
Klein et al., Proc. Natl. Acad. Sci. USA, 85:4305 (1988).
Kohler et al., Plant Mol. Biol., 29:1293 (1995).
Knauf, et al., Genetic Analysis of Host Range Expression by Agrobacterium In: Molecular Genetics of the Bacteria-Plant Interaction, Puhler, A. ed., Springer
Koziel et al., Biotechnology, 11: 194 (1993).
Kridl et al., Seed Science Research, 1:209 (1991).
Kriz et al., Mol. Gen. Genet., 207:90 (1987).
Kunkel, Proc. Natl. Acad. Sci. USA, 82:488 (1985).
Kunkel et al., Methods in Enzymol., 154:367 (1987).
Katz et al., J. Gen. Microbiol., 129, 1983
Keegan et al., Science, 231:699 (1986))
Klimyuk et al. (Plant J., 3:493-494, 1993
Koehl and Delarue, Curr. Opin. Struct. Biol., 6:222, 1996
Krysan et al., Plant Cell, 11:2283-2290, 1999
Langridge et al., Cell, 34:1015 (1983).
Lindstrom et al., Der. Genet., 11:160 (1990).
Liu et al., Plant J. 8:457-463 (1995).
Lommel et al., Virology, 81:382 (1991).
Lawton et al., Mol. Cell Biol., 7:335, 1987
Laufs et al., Proc. Natl. Acad. Sci., USA, 87:7752, 1990
Laursen et al., Plant Mol. Biol., 24:51, 1994
Lesyng. and McCammon, Pharmocol. Ther., 60:149, 1993
Li et al., Mol Gen Genet 261:58 (1999)).
Lindsey et al., Transgenic Res., 2:3347, 1993
Macejak et al., Nature, 353:90 (1991).
Mansson et al., Gen. Genet., 200:356 (1985).
Martinez et al., J. Mol. Biol., 208:551 (1989).
Matteucci et al. J Am Chem Soc 103: 3185 (1981).
McBride et al., Proc. Natl. Acad. Sci. USA, 91:7301 (1994).
McCabe et al., Bio/Technology, 6:923 (1988).
Meinkoth and Wahl, Anal. Biochem., 138:267 (1984).
Messing & Vierra, Gene, 19:259 (1982).
Mogen et al., Plant Cell 2:1261 (1990).
Munroe et al., Gene 91:151 (1990).
Murray et al., Nucleic Acids Res., 17:477 (1989).
Myers et al., CABIOS 4:11 (1988).
Maki et al., (“Procedures for Introducing Foreign DNA into Plants” in Methods in Plant Molecular Biology & Biotechnology, Glich et al. Eds. pp. 67-88 CRC Press, 1993
Michael J. Mol. Biol., 26:585, (1994)
Miyao et al. (DNA Res 3:233 (1996))
Moore et al., J. Mol. Biol., 272:336 (1997).
Murakami et al., Mol. Gen. Genet., 205:42 (1986)
Murata et al., FEBS Lett., 296:187, (1992).
Narang et al. Meth Enzymol 68: 90 (1979)
Needleman and Wunsch, J. Mol. Biol. 48:443 (1970).
Narang et al. (Meth Enzymol, 68: 90, (1979).
Negrotto et al., Plant Cell Reports 19, 798-803 (2000)
Niedz et al., Plant Cell Reports, 14: 403, (1995)
Ooms et al., Plasmid 7, 15-29 (1982).
Ow et al., Science, 234:856 (1986)
Odell et al., Nature, 313:810 (1985).
Ohtsuka et al., J. Biol. Chem., 260:2605 (1985).
Okamuro et al., Biochemistry of Plants, 15:1 (1989).
Paszkowski et al., EMBO J., 3:2717 (1984).
Pacciotti et al. Bio/Technology 3:241 (1985).
Park et al., J. Plant Biol. 38(4):365 (1985).
Pazour et al., J. Bacteriol. 174:4169-4174 (1992).
Pearson et al., Meth. Mol. Biol. 24:307 (1994).
Pearson and Lipman, Proc. Natl. Acad. Sci. 85:2444 (1988).
Perlak et al., Proc. Natl. Acad. Sci. USA, 88:3324 (1991).
Proudfoot, Cell 64:671 (1991).
Parinov et al., Plant Cell, 11:2263-2270, (1999).
Parinov and Sundaresan, Biotechnology, 11:157-161,(2000)
Pear et al., Plant Mol. Biol., 13:639, (1989)
Phillips et al. (“Cell-Tissue Culture and In-Vitro Manipulation” in Corn & Corn Improvement, 3rd Ed., Sprague et al., Eds., pp. 345-387. American Society of Agronomy Inc., (1988).
Poulsen et al., Mol. Gen. Genet., 205:193, (1986)
Prasher et al., Biochem. Biophys. Res. Comm., 126: 1259, (1985)
Quigley et al., J. Mol. Evol., 29:412 (1989).
Ralston et al., Genetics, 119:185 (1988).
Reina et al., Nucleic Acids Res., 18:6425 (1990).
Reina et al., Nucleic Acids Res., 18:7449 (1990).
Riggs et al., Proc. Natl. Acad. Sci. USA, 83:5602 (1986).
Rossolini et al., Mol. Cell. Probes, 8:91 (1994).
Roth et al., Nature Biotechnology, 16:939 (1998).
Ruiz et al., Plant Cell 10:937 (1998).
Refseth et al., Electrophoresis 18:1519 (1997)).
Rossi et al., Biophys. J., 80:480, (2001).
Skriver and Mundy, Plant Cell, 2:503 (1990)
Stalker et al., Science, 242:419 (1988).
Steifel et al., Plant Cell, 2:785 (1990)
Sussman et al., Plant Physiology, 124:1465-1467, (2000)
Surles et al., Protein Sci., 3:198, (1994)
Sutcliffe, Proc. Natl. Acad. Sci. USA, 75:3737, (1978)
Sambrook et al., Molecular Cloning: A Laboratory Manual (2d ed., Cold Spring Harbor Laboratory Press, Plainview, N.Y.) (1989).
Sanfacon et al., Genes Dev. 5:141 (1991).
Sanford et al., Particulate Science and Technology, 5:27 (1987).
Schwob et al., Plant J., 4:423 (1993).
Shimamoto et al., Nature, 338:274 (1989).
Simpson, Plant Mol. Biol., 19:699 (1985).
Skuzeski et al., Plant Molec. Biol., 15:65 (1990).
Slater et al., Plant Mol. Biol., 5:137 (1985).
Smith et al., Adv. Appl. Math. 2:482 (1981).
Smith et al., Planta 168:94 (1986).
Spencer et al., Theor Appl Genet, 79:625 (1990).
Staub et al., EMBO J., 12:601 (1993).
Staub et al., Plant Cell, 4:39 (1992).
Stemmer, Nature, 370:389 (1994).
Stemmer, Proc. Natl. Acad. Sci. USA, 91:10747 (1994).
Sukhapinda et al. Plant Mol. Biol. 8:209 (1987).
Sullivan et al., Mol. Gen. Genet., 215:431 (1989).
Svab et al., Proc. Natl. Acad. Sci. USA, 87:8526 (1990).
Svab et al., Proc. Natl. Acad. Sci. USA, 90:913 (1993).
Tamayo, P., et al., Proc. Natl. Acad. Sci. USA 96, 2907-12, (1999).
Tanksley and McCouch, Science 277:1063 (1997)).
Thillet et al., J. Biol. Chem., 263:12500, 1988
Thompson et al., EMBO J., 6:2519, 1987
Tisser et al., Plant Cell, 11:1841-1852, 1999
Tomic et al. NAR, 12:1656, 1990
Tomes et al. “Direct DNA transfer into intact plant cells via microprojectile bombardment,” Plant Cell, Tissue and Organ Culture: Fundamental Methods; Springer Verlag, Gamborg and Phillips,Eds., (1995)
Triezenberg et al., Genes Dev., 2:718-729, (1988)
Turner et al., Molecular Biotechnology, 3:225 (1995).
Twell et al., Plant Physiol., 91:1270,(1989)
Tijssen, Laboratory Techniques in Biochemistry and Molecular Biology-Hybridization with Nucleic Acid Probes, part I chapter 2 “Overview of principles of hybridization and the strategy of nucleic acid probe assays” Elsevier, New York (1993).
Turner et al., Molecular Biotechnology, 3:225 (1995).
Urdea et al. Proc Natl Acad 80: 7461 (1981).
Upender et al., Biotechniques, 18:29, 1995
Van Tunen et al., EMBO J., 7:1257 (1988).
Vasil et al., Biotechnology, 11:1553 (1993).
Vasil et al., Plant Physiol. 01:1575 (1989).
Vodkin, Prog. Clin. Biol. Res., 138:87 (1983).
Vogel et al., EMBO J., 11:157 (1992).
Wang et al., Molec. Cell. Biol., 12:3399 (1992).
Walker and Gaastra, eds., Techniques in Molecular Biology, MacMillan Publishing Company, New York (1983).
Wandelt et al., Nucleic Acids Res., 17:2354 (1989).
Waterman, M. S. Introduction to Computational Biology: Maps, sequences and genomes. Chapman & Hall. London (1995).
Weeks et al., Plant Physiol., 102:1077 (1993).
Weissinger et al., Annual Rev. Genet., 22:421 (1988).
Watson and Rarnstad, Corn: Chemistry and Technology American Assn. Cereal Chemists, (1987)
Wang et al., Molec. Cell Biol., 12:3399, (1992)
Wolter et al., EMBO J., 11:4685, (1992)
Wenzler et al., Plant Mol. Biol., 13:347 (1989).
White et al., Nucl Acids Res, 18:1062 (1990).
Yamamoto et al., Nucleic Acids Res., 18:7449 (1990).
Yang et al., Plant Mol. Biol., 38:1201 (1998).
Yanagisawa et al., Plant J., 17:209-214 (1999).
Yadav et al., Proc. Natl. Acad. Sci. USA. 79: 6322-6326 (1982).
Yang et al. (Mol Gen Genet 245:187 (1994))
Yannisch-Perron et al., Gene 33: 103-119 (1985).
Zhu, T., et al., Plant Physiol. 124, 1472-1476 (2000).
Zhu, T., Et al. Plant Physiol. Biochem. 39, 221-242. (2001a)
Zhu, T., et al. Gene Expression Microarrays: Improvements and Applications Towards Agricultural Gene Discovery. Journal of the Association for Laboratory Automation. 6: in press. (2001b).
Zhang et al., Proc. Natl. Acad. Sci. USA, 94:4504 (1997).
Zietkiewicz et al., Genomics 20:176 (1994)
Zukowsky et al., Proc. Natl. Acad. Sci. USA, 80:1101, (1983)

1. An isolated polynucleotide comprising a nucleic acid sequence selected from the group consisting of SEQ ID NO.: 1-70 or a functional fragment thereof.

2. The isolated polynucleotide of claim 1, wherein said isolated polynucleotide encodes a transcription factor.

3. The isolated polynucleotide of claim 1, wherein said polynucleotide encodes a transcription factor functional in a monocot.

4. The isolated polynucleotide of claim 1 wherein said polynucleotide encodes a transcription factor functional in a cereal.

5. The isolated polynucleotide of claim 1 wherein said polynucleotide encodes a transcription factor functional in rice.

6. The isolated polynucleotide of claim 1 wherein said polynucleotide encodes a AP2/EREBP transcription factor.

7. The isolated polynucleotide of claim 6, wherein said polynucleotide is selected from the group consisting of SEQ ID NO.: 20, 23, 33, 36, 40, 44 and 47.

8. The isolated polynucleotide of claim 1 wherein said polynucleotide encodes a bZIP transcription factor.

9. The isolated polynucleotide of claim 8, wherein the polynucleotide is selected from the group consisting of SEQ ID NO.: 6, 27, 45 and 48.

10. The isolated polynucleotide of claim 1, wherein said polynucleotide encodes a Dof zinc finger protein.

11. The isolated polynucleotide of claim 10, wherein said polynucleotide is selected from SEQ ID NO: 1 or 17.

12. The isolated polynucleotide of claim 1, wherein said polynucleotide encodes a homeodomain-leucine zipper.

13. The isolated polynucleotide of claim 12, wherein said polynucleotide is selected from the group consisting of SEQ ID NO.18, 30, 32, 52, and 53.

14. The isolated polynucleotide of claim 1, wherein said polynucleotide encodes an IAA type transcription factor.

15. The isolated polynucleotide of claim 14, wherein said polynucleotide is SEQ ID NO.: 31 or 42.

16. The isolated polynucleotide of claim 1, wherein said polynucleotide encodes a Myb transcription factor.

17. The isolated polynucleotide of claim 16, wherein said polynucleotide is selected from the group consisting of SEQ ID NO.: 5, 7, 10, 14, 15, 16, 21, 25, 26, 34, 35 37, 50 and 51.

18. The isolated polynucleotide of claim 1, wherein said polynucleotide encodes a WRKY transcription factor.

19. The isolated polynucleotide of claim 18, wherein said polynucleotide is selected from the group consisting of SEQ ID NO.: 2 and 43.

20. The isolated polynucleotide of claim 1, wherein said polynucleotide encodes a zinc finger protein.

21. The isolated polynucleotide of claim 20, wherein said polynucleotide is selected from the group consisting of SEQ ID NO.: 8, 11, 13 and 22.

22. An isolated polynucleotide which is complementary to the polynucleotide of claim 1.

23. An isolated polynucleotide that is at least 70% identical to a polynucleotide of claim 1.

24. An isolated polynucleotide that is at least 90% identical to the polynucleotide of claim 1.

25. An isolated polynucleotide of at least 15 nucleotides in length that hybridizes under stringent conditions to a polynucleotide of claim 1.

26. The isolated polynucleotide of claim 25, wherein said polynucleotide encodes a transcription factor or a functional fragment thereof.

27. An isolated polypeptide comprising an amino acid sequence encoded by a polynucleotide of claim 1.

28. The isolated polypeptide of claim 27, wherein said isolated polypeptide comprises a transcription factor.

29. The isolated polypeptide of claim 28, wherein said polypeptide encodes a transcription factor functional in a monocot.

30. The isolated polypeptide of claim 29 wherein said polypeptide encodes a transcription factor functional in a cereal.

31. The isolated polypeptide of claim 30 wherein said polypeptide encodes a transcription factor functional in rice.

32. The isolated polypeptide of claim 27 wherein said polypeptide comprises a AP2/EREBP transcription factor.

33. The isolated polypeptide of claim 32, wherein said polypeptide has an amino acid sequence encoded by a polynucleotide selected from the group consisting of SEQ ID NO.: 20, 23, 33, 36, 40, 44 and 47.

34. The isolated polypeptide of claim 27 wherein said polypeptide comprises a bZIP transcription factor.

35. The isolated polypeptide of claim 34, wherein wherein said polypeptide has an amino acid sequence encoded by a polynucleotide selected from the group consisting of SEQ ID NO.: 6, 27, 45 and 48.

36. The isolated polypeptide of claim 27, wherein said polypeptide comprises a Dof zinc finger protein.

37. The isolated polypeptide of claim 36, wherein said polypeptide has an amino acid sequence encoded by a polynucleotide selected from SEQ ID NO: 1 or 17.

38. The isolated polypeptide of claim 27, wherein said polypeptide comprises a homeodomain-leucine zipper.

39. The isolated polypetide of claim 38, wherein said polypeptide has an amino acid sequence encoded by a polynucleotide selected from the group consisting of SEQ ID NO.18, 30, 32, 52, and 53.

40. The isolated polypeptide of claim 27, wherein said polypeptide comprises an IAA type transcription factor.

41. The isolated polypeptide of claim 40, wherein said polypeptide is encoded by SEQ ID NO.: 31 or 42.

42. The isolated polypeptide of claim 27, wherein said polypeptide comprises a Myb transcription factor.

43. The isolated polypeptide of claim 42, wherein said polypeptide has an amino acid sequence encoded by a polynucleotide selected from the group consisting of SEQ ID NO.: 5, 7, 10, 14, 15, 16, 21, 25, 26, 34, 35 37, 50 and 51.

44. The isolated polypeptide of claim 27, wherein said polypeptide comprises a WRKY transcription factor.

45. The isolated polypeptide of claim 44, wherein said polypeptide has an amino acid sequence encoded by a polynucleotide selected from the group consisting of SEQ ID NO.: 2 and 43.

46. The isolated polypeptide of claim 27, wherein said polypeptide comprises a zinc finger protein.

47. The isolated polypeptide of claim 46, wherein said polypeptide has an amino acid sequence encoded by a polynucleotide selected from the group consisting of SEQ ID NO.: 8, 11, 13 and 22.

48. A recombinant vector comprising a polynucleotide of claim 1.

49. The vector of claim 48, wherein said vector is a cloning vector

50. The vector of claim 48, wherein said vector is an expression vector.

51. An expression cassette comprising as operably linked components, a promoter, a polynucleotide of claim 1 and a termination sequence.

52. A host cell comprising a vector of claim 48.

53. A host cell comprising an expression cassette of claim 51.

54. The host cell of claim 52 wherein said host cell is a bacterial cell, a yeast cell, an animal cell or a plant cell.

55. The host cell of claim 54, wherein said host cell is a plant cell.

56. The host cell of claim 55, wherein said plant cell is a monocot.

57. The host cell of claim 56, wherein said plant cell is from a cereal.

58. The host cell of claim 57, wherein said plant cell is a rice plant cell.

59. A plant comprising a host cell of claim 52.

60. A plant comprising a recombinant nucleic acid construct comprising a polynucleotide of claim 1.

61. The plant of claim 59 wherein said plant is a dicot.

62. The plant of claim 59, wherein said plant is a monocot.

63. The plant of claim 62, wherein said monocot is a cereal.

64. The plant of claim 63, wherein said cereal is rice.

65. The plant of claim 62, wherein said plant is selected from the group consisting of maize, soybean, barley, alfalfa, sunflower, canola, soybean, cotton, peanut, sorghum, tobacco, sugarbeet, wheat, and rice.

66. A computer-readable medium having stored thereon a data structure comprising a nucleic acid sequence having at least 70% sequence identity to a nucleotide sequence selected from the group consisting of SEQ ID NO.: 1-70, or the complement thereof; and
a module receiving the nucleic acid sequence which compares the nucleic acid sequence to at least one other nucleic acid sequence.

67. The computer-readable medium of claim 66, wherein the medium is selected from the group consisting of magnetic tape, optical disk, compact disc, random access memory, volatile memory, non-volatile memory and bubble memory.

68. A computer-readable medium having stored thereon computer executable instruction for performing a method comprising receiving a nucleic acid sequence having at least 70% sequence identity to a nucleotide sequence selected from the group consisting of SEQ ID NO.: 1-70 or the complement thereof; and
comparing the nucleic acid sequence to at least one other nucleic acid sequence.

69. The computer-readable medium of claim 68, wherein the compute-readable medium is selected from the group consisting of magnetic tape, optical disk, compact disc, random access memory, volatile memory, non-volatile memory and bubble memory.

70. A method for altering resistance or tolerance of a plant to a stress comprising transforming plant cells with an expression cassette containing a polynucleotide encoding a polypeptide that is identical or substantially similar to a polypeptide encoded by a nucleotide sequence selected from the group consisting of SEQ ID NO.: 1-70; and regenerating the transformed plant cells to provide a transformed plant, wherein the transformed plant expresses the polypeptide contained in said expression cassette in an amount sufficient to alter the resistance or tolerance of the plant to a stress relative to a plant which was not transformed with said expression cassette.

71. A method for altering a biological pathway in a plant regulated by a transcription factor comprising, transforming plant cells with an expression cassette containing a polynucleotide encoding a polypeptide that is identical or substantially similar to a polypeptide encoded by a nucleotide sequence selected from the group consisting of SEQ ID NO.: 1-70; and regenerating the transformed plant cells to provide a transformed plant, wherein the transformed plant expresses the polypeptide contained in said expression cassette in an amount sufficient to alter the biological pathway.

72. A method for altering the expression of one or more genes in a plant comprising, transforming plant cells with an expression cassette containing a polynucleotide encoding a polypeptide that is identical or substantially similar to a polypeptide encoded by a nucleotide sequence selected from the group consisting of SEQ ID NO.: 1-70; and regenerating the transformed plant cells to provide a transformed plant, wherein the transformed plant expresses the polypeptide contained in said expression cassette in an amount sufficient alter expression of said gene or genes.

73. The method of claim 70, wherein said polynucleotide contained in said expression cassette is in a sense orientation.

74. The method of claim 70, wherein said polynucleotide contained in said expression cassette is in an antisense orientation.

75. The method of claim 70 wherein said plant is a dicot.

76. The method of claim 70, wherein said plant is a monocot.

77. The method of claim 76, wherein said monocot is a cereal.

78. The method of claim 77, wherein said cereal is rice.

79. The method of claim 76 wherein said plant is selected from the group consisting of maize, soybean, barley, alfalfa, sunflower, canola, soybean, cotton, peanut, sorghum, tobacco, sugarbeet, wheat, and rice.

80. The method of claim 70, wherein said stress is drought stress.

81. The method of claim 80, wherein said nucleotide sequence is selected from the group consisting of SEQ ID NO.: 1, 10, 11, 17, 20, 26, 27 and 32.

82. The method of claim 70, wherein said stress is cold stress.

83. The method of claim 82, wherein said nucleotide sequence is selected from the group consisting of SEQ ID NO.: 3, 4, 20, 42, 45 and 46.

84. The method of claim 70, wherein said stress is osmotic stress.

85. The method of claim 84, wherein said nucleotide sequence is selected from the group consisting of SEQ ID NO.: 4, 20, 26, 32 and 45.

86. The method of claim 70, wherein said stress is salt stress.

87. The method of claim 86, wherein said nucleotide sequence is selected from the group consisting of 20, 26 and 45.

88. The progeny of a plant of claim 59.

89. The progeny of claim 88, wherein said progeny are hybrid plants.

90. Seeds from a plant of claim 59.

91. A uniform population of plants from claim 59.

92. A uniform population of plants from claim 88.
